SEQUENCE LISTING (207 sequences)

SEQ ID NO: 1 - protein, 251 aa, Escherichia coli [sequence omitted]

SEQ ID NO: 2 - DNA, 756 bp, Escherichia coli
  source 1..756: mol_type = other DNA
atggcaacggttaataaacaagccattgcagcggcatttggtcgggcagccgcacactatgagcaacatgcagatctacagcgccagagtgctgacgccttactggcaatgcttccacagcgtaaatacacccacgtactggacgcgggttgtggacctggctggatgagccgccactggcgggaacgtcacgcgcaggtgacggccttagatctctcgccgccaatgcttgttcaggcacgccagaaggatgccgcagaccattatctggcgggagatatcgaatccctgccgttagcgactgcgacgttcgatcttgcatggagcaatctcgcagtgcagtggtgcggtaatttatccacggcactccgcgagctgtatcgggtggtgcgccccaaaggcgtggtcgcgtttaccacgctggtgcagggatcgttacccgaactgcatcaggcgtggcaggcggtggacgagcgtccgcatgctaatcgctttttaccgccagatgaaatcgaacagtcgctgaacggcgtgcattatcaacatcatattcagcccatcacgctgtggtttgatgatgcgctcagtgccatgcgttcgctgaaaggcatcggtgccacgcatcttcatgaagggcgcgacccgcgaatattaacgcgttcgcagttgcagcgattgcaactggcctggccgcaacagcaggggcgatatcctctgacgtatcatctttttttgggagtgattgctcgtgagtaa

SEQ ID NO: 3 - protein, 256 aa, Escherichia coli
MNNIWWQTKGQGNVHLVLLHGWGLNAEVWRCIDEELSSHFTLHLVDLPGFGRSRGFGALSLADMAEAVLQQAPDKAIWLGWSLGGLVASQIALTHPERVQALVTVASSPCFSARDEWPGIKPDVLAGFQQQLSDDFQRTVERFLALQTMGTETARQDARALKKTVLALPMPEVDVLNGGLEILKTVDLRQPLQNVSMPFLRLYGYLDGLVPRKVVPMLDKLWPHSESYIFAKAAHAPFISHPAEFCHLLVALKQRV

SEQ ID NO: 4 - DNA, 771 bp, Escherichia coli
  source 1..771: mol_type = other DNA
atgaataacatctggtggcagaccaaaggtcaggggaatgttcatcttgtgctgctgcacggatggggactgaatgccgaagtgtggcgttgcattgacgaggaacttagctcgcattttacgctgcaccttgttgacctgcccggcttcgggcgtagccggggatttggtgcgctgtcacttgctgatatggccgaagccgtgctgcaacaggcacctgataaagccatttggttaggctggagtctgggcgggctggtggcaagccagattgcgttaacccatcccgagcgtgttcaggcgctggtcaccgtggcgtcgtcaccttgttttagtgctcgtgacgagtggccggggataaaaccggacgtgctggcgggatttcagcagcaactcagtgatgattttcagcgtacagtggagcggttcctggcgttacaaaccatggggactgaaacggcgcgccaggatgcgcgggcgttgaagaaaaccgttctggcgttaccgatgccggaggttgacgtgcttaatggcgggctggaaatcctgaaaacggtcgatctccgtcagccgctgcaaaacgtgtccatgccgtttttgcgattgtatggctatctcgacggtctggtgccgcgcaaagtggtgccgatgctggataaactttggcctcacagcgaatcatatatcttcgccaaagcggcccatgcgccatttatttcgcatccggccgagttttgtcacctgctggtggcgttgaagcagagggtgtag

SEQ ID NO: 5 - protein, 384 aa, Escherichia coli [sequence omitted]

SEQ ID NO: 6 - DNA, 1155 bp, Escherichia coli
  source 1..1155: mol_type = other DNA
atgagctggcaggagaaaatcaacgcggcgctcgatgcgcggcgtgctgccgatgccctgcgtcgccgttatccggtggcgcaaggagccggacgctggctggtggcggatgatcgccagtatctgaacttttccagtaacgattatttaggtttaagccatcatccgcaaattatccgtgcctggcagcagggggcggagcaatttggcatcggtagcggcggctccggtcacgtcagcggttatagcgtggtgcatcaggcactggaagaagagctggccgagtggcttggctattcgcgggcactgctgtttatctctggtttcgccgctaatcaggcagttattgccgcgatgatggcgaaagaggaccgtattgctgccgaccggcttagccatgcctcattgctggaagctgccagtttaagcccgtcgcagcttcgccgttttgctcataacgatgtcactcatttggcgcgattgcttgcttccccctgtccggggcagcaaatggtggtgacagaaggcgtgttcagcatggacggcgatagtgcgccactggcggaaatccagcaggtaacgcaacagcacaatggctggttgatggtcgatgatgcccacggcacgggcgttatcggggagcaggggcgcggcagctgctggctgcaaaaggtaaaaccagaattgctggtagtgacttttggcaaaggatttggcgtcagcggggcagcggtgctttgctccagtacggtggcggattatctgctgcaattcgcccgccaccttatctacagcaccagtatgccgcccgctcaggcgcaggcattacgtgcgtcgctggcggtcattcgcagtgatgagggtgatgcacggcgcgaaaaactggcggcactcattacgcgttttcgtgccggagtacaggatttgccgtttacgcttgctgattcatgcagcgccatccagccattgattgtcggtgataacagccgtgcgttacaactggcagaaaaactgcgtcagcaaggctgctgggtcacggcgattcgcccgccaaccgtacccgctggtactgcgcgactgcgcttaacgctaaccgctgcgcatgaaatgcaggatatcgaccgtctgctggaggtgctgcatggcaacggttaa

SEQ ID NO: 7 - protein, 429 aa, Escherichia coli
MTTDDLAFDQRHIWHPYTSMTSPLPVYPVVSAEGCELILSDGRRLVDGMSSWWAAIHGYNHPQLNAAMKSQIDAMSHVMFGGITHAPAIELCRKLVAMTPQPLECVFLADSGSVAVEVAMKMALQYWQAKGEARQRFLTFRNGYHGDTFGAMSVCDPDNSMHSLWKGYLPENLFAPAPQSRMDGEWDERDMVGFARLMAAHRHEIAAVIIEPIVQGAGGMRMYHPEWLKRIRKICDREGILLIADEIATGFGRTGKLFACEHAEIAPDILCLGKALTGGTMTLSATLTTREVAETISNGEAGCFMHGPTFMGNPLACAAANASLAILESGDWQQQVADIEVQLREQLAPARDAEMVADVRVLGAIGVVETTHPVNMAALQKFFVEQGVWIRPFGKLIYLMPPYIILPQQLQRLTAAVNRAVQDETFFCQ

SEQ ID NO: 8 - DNA, 1290 bp, Escherichia coli
  source 1..1290: mol_type = other DNA
atgacaacggacgatcttgcctttgaccaacgccatatctggcacccatacacatccatgacctcccctctgccggtttatccggtggtgagcgccgaaggttgcgagctgattttgtctgacggcagacgcctggttgacggtatgtcgtcctggtgggcggcgatccacggctacaatcacccgcagcttaatgcggcgatgaagtcgcaaattgatgccatgtcgcatgtgatgtttggcggtatcacccatgcgccagccattgagctgtgccgcaaactggtggcgatgacgccgcaaccgctggagtgcgtttttctcgcggactccggttccgtagcggtggaagtggcgatgaaaatggcgttgcagtactggcaagccaaaggcgaagcgcgccagcgttttctgaccttccgcaatggttatcatggcgatacctttggcgcgatgtcggtgtgcgatccggataactcaatgcacagtctgtggaaaggctacctgccagaaaacctgtttgctcccgccccgcaaagccgcatggatggcgaatgggatgagcgcgatatggtgggctttgcccgcctgatggcggcgcatcgtcatgaaatcgcggcggtgatcattgagccgattgtccagggcgcaggcgggatgcgcatgtaccatccggaatggttaaaacgaatccgcaaaatatgcgatcgcgaaggtatcttgctgattgccgacgagatcgccactggatttggtcgtaccgggaaactgtttgcctgtgaacatgcagaaatcgcgccggacattttgtgcctcggtaaagccttaaccggcggcacaatgaccctttccgccacactcaccacgcgcgaggttgcagaaaccatcagtaacggtgaagccggttgctttatgcatgggccaacttttatgggcaatccgctggcctgcgcggcagcaaacgccagcctggcgattctcgaatctggcgactggcagcaacaggtggcggatattgaagtacagctgcgcgagcaacttgcccccgcccgtgatgccgaaatggttgccgatgtgcgcgtactgggggccattggcgtggtcgaaaccactcatccggtgaatatggcggcgctgcaaaaattctttgtcgaacagggtgtctggatccggccttttggcaaactgatttacctgatgccgccctatattattctcccgcaacagttgcagcgtctgaccgcagcggttaaccgcgcggtacaggatgaaacatttttttgccaataa

SEQ ID NO: 9 - protein, 448 aa, Bacillus subtilis
MTHDLIEKSKKHLWLPFTQMKDYDENPLIIESGTGIKVKDINGKEYYDGFSSVWLNVHGHRKKELDDAIKKQLGKIAHSTLLGMTNVPATQLAETLIDISPKKLTRVFYSDSGAEAMEIALKMAFQYWKNIGKPEKQKFIAMKNGYHGDTIGAVSVGSIELFHHVYGPLMFESYKAPIPYVYRSESGDPDECRDQCLRELAQLLEEHHEEIAALSIESMVQGASGMIVMPEGYLAGVRELCTTYDVLMIVDEVATGFGRTGKMFACEHENVQPDLMAAGKGITGGYLPIAVTFATEDIYKAFYDDYENLKTFFHGHSYTGNQLGCAVALENLALFESENIVEQVAEKSKKLHFLLQDLHALPHVGDIRQLGFMCGAELVRSKETKEPYPADRRIGYKVSLKMRELGMLTRPLGDVIAFLPPLASTAEELSEMVAIMKQAIHEVTSLED

SEQ ID NO: 10 - DNA, 1347 bp, Bacillus subtilis
  source 1..1347: mol_type = other DNA
atgactcatgatttgatagaaaaaagtaaaaagcacctctggctgccatttacccaaatgaaagattatgatgaaaaccccttaatcatcgaaagcgggactggaatcaaagtcaaagacataaacggcaaggaatactatgacggtttttcatcggtttggcttaatgtccacggacaccgcaaaaaagaactagatgacgccataaaaaaacagctcggaaaaattgcgcactccacgttattgggcatgaccaatgttccagcaacccagcttgccgaaacattaatcgacatcagcccaaaaaagctcacgcgggtcttttattcagacagcggcgcagaggcgatggaaatagccctaaaaatggcgtttcagtattggaagaacatcgggaagcccgagaaacaaaaattcatcgcaatgaaaaacgggtatcacggtgatacgattggcgccgtcagtgtcggttcaattgagctttttcaccacgtatacggcccgttgatgttcgagagttacaaggccccgattccttatgtgtatcgttctgaaagcggtgatcctgatgagtgccgtgatcagtgcctccgagagcttgcacagctgcttgaggaacatcatgaggaaattgccgcgctttccattgaatcaatggtacaaggcgcgtccggtatgatcgtgatgccggaaggatatttggcaggcgtgcgcgagctatgtacaacatacgatgtcttaatgatcgttgatgaagtcgctacaggctttggccgtacaggaaaaatgtttgcgtgcgagcacgagaatgtccagcctgatctgatggctgccggtaaaggcattacaggaggctatttgccaattgccgttacgtttgccactgaagacatctataaggcattctatgatgattatgaaaacctaaaaacctttttccatggccattcctatacaggcaatcagcttggctgtgcggttgcgcttgaaaatctggcattatttgaatctgaaaacattgtggaacaagtagcggaaaaaagtaaaaagctccattttcttcttcaagatctgcacgctcttcctcatgttggggatattcggcagcttggctttatgtgcggtgcagagcttgtacgatcaaaggaaactaaagaaccttacccggctgatcggcggattggatacaaagtttccttaaaaatgagagagttaggaatgctgacaagaccgcttggggacgtgattgcatttcttcctcctcttgccagcacagctgaagagctctcggaaatggttgccattatgaaacaagcgatccacgaggttacgagccttgaagattga

SEQ ID NO: 11 - protein, 225 aa, Escherichia coli
MSKRYFVTGTDTEVGKTVASCALLQAAKAAGYRTAGYKPVASGSEKTPEGLRNSDALALQRNSSLQLDYATVNPYTFAEPTSPHIISAQEGRPIESLVMSAGLRALEQQADWVLVEGAGGWFTPLSDTFTFADWVTQEQLPVILVVGVKLGCINHAMLTAQVIQHAGLTLAGWVANDVTPPGKRHAEYMTTLTRMIPAPLLGEIPWLAENPENAATGKYINLALL

SEQ ID NO: 12 - DNA, 678 bp, Escherichia coli
  source 1..678: mol_type = other DNA
atgagtaaacgttattttgtcaccggaacggataccgaagtggggaaaactgtcgccagttgtgcacttttacaagccgcaaaggcagcaggctaccggacggcaggttataaaccggtcgcctctggcagcgaaaagaccccggaaggtttacgcaatagcgacgcgctggcgttacagcgcaacagcagcctgcagctggattacgcaacagtaaatccttacaccttcgcagaacccacttcgccgcacatcatcagcgcgcaagagggcagaccgatagaatcattggtaatgagcgccggattacgcgcgcttgaacaacaggctgactgggtgttagtggaaggtgctggcggctggtttacgccgctttctgacactttcacttttgcagattgggtaacacaggaacaactgccggtgatactggtagttggtgtgaaactcggctgtattaatcacgcgatgttgactgcacaggtaatacaacacgccggactgactctggcgggttgggtggcgaacgatgttacgcctccgggaaaacgtcacgctgaatatatgaccacgctcacccgcatgattcccgcgccgctgctgggagagatcccctggcttgcagaaaatccagaaaatgcggcaaccggaaagtacataaaccttgccttgttgtag

SEQ ID NO: 13 - protein, 395 aa, Bacillus subtilis
MTIASSTASSEFLKNPYSFYDTLRAVHPIYKGSFLKYPGWYVTGYEETAAILKDARFKVRTPLPESSTKYQDLSHVQNQMMLFQNQPDHRRLRTLASGAFTPRTTESYQPYIIETVHHLLDQVQGKKKMEVISDFAFPLASFVIANIIGVPEEDREQLKEWAASLIQTIDFTRSRKALTEGNIMAVQAMAYFKELIQKRKRHPQQDMISMLLKGREKDKLTEEEAASTCILLAIAGHETTVNLISNSVLCLLQHPEQLLKLRENPDLIGTAVEECLRYESPTQMTARVASEDIDICGVTIRQGEQVYLLLGAANRDPSIFTNPDVFDITRSPNPHLSFGHGHHVCLGSSLARLEAQIAINTLLQRMPSLNLADFEWRYRPLFGFRALEELPVTFE

SEQ ID NO: 14 - DNA, 1188 bp, Bacillus subtilis
  source 1..1188: mol_type = other DNA
gtgacaattgcatcgtcaactgcatcttctgagtttttgaaaaacccatattctttttacgacacattgcgagctgttcatcctatctataaagggagtttcttaaaatacccgggctggtatgtcacaggatatgaagaaacggctgctattttgaaagatgcgagattcaaagtccgcaccccgctgcctgagagctcaaccaaatatcaggacctttcacatgtgcaaaatcaaatgatgctgtttcagaaccagcctgatcatagacgattgcggacgcttgccagcggagcgtttacgccgagaacgacagagagttatcagccgtatatcattgaaactgtccatcatttgcttgatcaagtgcaaggtaaaaaaaagatggaggtcatttcggactttgcttttcctttagcaagttttgtcatagctaacattataggtgtaccggaggaagatagggagcaattaaaggagtgggctgcgagtctcattcaaacgattgattttacccgctcaagaaaggcattaacagagggcaatattatggctgtgcaggctatggcatatttcaaagagctgattcaaaagagaaaacgccaccctcaacaggatatgatcagcatgctcttgaaggggagagaaaaggataagctgacggaagaggaggcggcatctacgtgcatattgctggcgatcgccggacatgagacaacggtcaatctcatcagcaattcagtcctttgtctgctgcagcatccagaacagcttttgaaactgagagaaaatccagatcttattggtaccgcagtcgaggaatgtttacgctatgaaagccccacgcaaatgacagccagagttgcgtcagaggatattgacatctgcggggtgacgatccgtcaaggagaacaagtctatcttttgttaggagcggctaatcgagaccctagcatattcacgaaccccgatgtcttcgatattacgagaagtcctaatccgcatctttcattcgggcatggccatcatgtttgcttagggtcctcgctggcacgattagaagcgcaaattgcgattaacactcttctgcagcgaatgcccagccttaatcttgcggattttgaatggcggtatcggccgctttttggatttcgggcgcttgaggagctgccggtgacttttgaataa

SEQ ID NO: 15 - protein, 259 aa, Bacillus subtilis
MMQEETFYSVRMRASMNGSHEDGGKHISGGERLIPFHEMKHTVNALLEKGLSHSRGKPDFMQIQFEEVHESIKTIQPLPVHTNEVSCPEEGQKLARLLLEKEGVSRDVIEKAYEQIPEWSDVRGAVLFDIHTGKRMDQTKEKGVRVSRMDWPDANFEKWALHSHVPAHSRIKEALALASKVSRHPAVVAELCWSDDPDYITGYVAGKKMGYQRITAMKEYGTEEGCRVFFIDGSNDVNTYIHDLEKQPILIEWEEDHDS

SEQ ID NO: 16 - DNA, 777 bp, Bacillus subtilis
  source 1..777: mol_type = other DNA
atgcaagaagaaactttttatagtgtcagaatgagggcttcaatgaatggatctcatgaagacggcggaaagcatatatccggcggagaacggcttattcctttccatgagatgaagcatacagtcaatgctttattagaaaaagggttatcccattcaagaggaaaacctgattttatgcaaattcaatttgaagaggtacatgaatcgataaaaaccattcagccattgcctgtgcatacgaatgaagtgagctgcccggaagaaggacaaaagcttgcccgattgttattggaaaaagaaggcgtttcacgagacgtgattgaaaaagcatatgaacaaatccctgaatggtcagatgtcaggggtgcggtgttgtttgatattcatacaggcaagcgaatggatcaaacaaaagaaaaaggggtgcgggtctccagaatggattggccggacgctaattttgaaaaatgggcgcttcacagtcacgtgccagctcattcaagaataaaagaggcccttgcgctcgcttcaaaggtaagccggcacccggcagtcgttgcagaattatgctggtcggacgatccggattacataacaggctatgttgcgggtaagaaaatgggctatcagcgtattacagcaatgaaagaatacgggactgaagagggctgccgagtcttttttattgatggatccaatgatgtaaacacgtacatacatgacctggagaagcagcctattttaatagagtgggaggaagatcatgactcatga

SEQ ID NO: 17 - protein, 162 aa, Escherichia coli
MRLTSKGRYAVTAMLDVALNSEAGPVPLADISERQGISLSYLEQLFSRLRKNGLVSSVRGPGGGYLLGKDASSIAVGEVISAVDESVDATRCQGKGGCQGGDKCLTHALWRDLSDRLTGFLNNITLGELVNNQEVLDVSGRQHTHDAPRTRTQDAIDVKLRA

SEQ ID NO: 18 - DNA, 489 bp, Escherichia coli
  source 1..489: mol_type = other DNA
atgagactgacatctaaagggcgctatgccgtgaccgcaatgcttgacgttgcgctcaactctgaagcgggcccggtaccgttggctgatatttccgaacgtcagggaatttccctttcttatctggaacaactgttttcccgtctgcgtaaaaatggtctggtttccagcgtacgtggaccaggcggtggttatctgttaggcaaagatgccagcagcatcgccgttggcgaagtaattagcgccgttgacgaatctgtagatgccacccgttgtcagggtaaaggcggctgccagggcggcgataaatgcctgacccacgcgctgtggcgtgatttgagcgaccgtctcaccggttttctcaacaacattactttaggcgaactggttaataaccaggaagtgctggatgtgtctggtcgtcagcatactcacgacgcgccacgcacccgcacacaagacgcgatcgacgttaagttacgcgcttaa

SEQ ID NO: 19 - protein, 162 aa, Shigella sonnei
MRLTSKGRYAVTAMLDVALNSEAGPVPLADISERQGISLSYLEQLFSRLRKNGLVSSVRGPGGGYLLGKDASSIAVGEVISAVDESVDATRCQGKGGCQGGDKCLTHALWRDLSDRLTGFLNNITLGELVNNQEVLDVSGRQHTHDAPRTRILDAIDVKLRA

SEQ ID NO: 20 - DNA, 486 bp, Shigella sonnei
  source 1..486: mol_type = other DNA
atgcgcctgaccagcaaaggccgctatgcggtgaccgcgatgctggatgtggcgctgaacagcgaagcgggcccggtgccgctggcggatattagcgaacgccagggcattagcctgagctatctggaacagctgtttagccgcctgcgcaaaaacggcctggtgagcagcgtgcgcggcccgggcggcggctatctgctgggcaaagatgcgagcagcattgcggtgggcgaagtgattagcgcggtggatgaaagcgtggatgcgacccgctgccagggcaaaggcggctgccagggcggcgataaatgcctgacccatgcgctgtggcgcgatctgagcgatcgcctgaccggctttctgaacaacattaccctgggcgaactggtgaacaaccaggaagtgctggatgtgagcggccgccagcatacccatgatgcgccgcgcacccgcattctggatgcgattgatgtgaaactgcgcgcg

SEQ ID NO: 21 - protein, 163 aa, Citrobacter pasteurii
MRLTSKGRYAVTAMLDVALNSETGPVPLADISERQGISLSYLEQLFSRLRKNGLVSSVRGPGGGYLLGKDASSIAVGEVISAVDESVDATRCQGKGGCQGGDKCLTHALWRDLSDRLTGFLNNITLGELVNNQEVLDVSGRQHTHDAPRTNRAQDAIDVKLRA

SEQ ID NO: 22 - DNA, 489 bp, Citrobacter pasteurii
  source 1..489: mol_type = other DNA
atgcgcctgaccagcaaaggccgctatgcggtgaccgcgatgctggatgtggcgctgaacagcgaaaccggcccggtgccgctggcggatattagcgaacgccagggcattagcctgagctatctggaacagctgtttagccgcctgcgcaaaaacggcctggtgagcagcgtgcgcggcccgggcggcggctatctgctgggcaaagatgcgagcagcattgcggtgggcgaagtgattagcgcggtggatgaaagcgtggatgcgacccgctgccagggcaaaggcggctgccagggcggcgataaatgcctgacccatgcgctgtggcgcgatctgagcgatcgcctgaccggctttctgaacaacattaccctgggcgaactggtgaacaaccaggaagtgctggatgtgagcggccgccagcatacccatgatgcgccgcgcaccaaccgcgcgcaggatgcgattgatgtgaaactgcgcgcg

SEQ ID NO: 23 - protein, 163 aa, Enterobacter timonensis
MRLTSKGRYAVTAMLDVALNSEAGPVPLADISERQGISLSYLEQLFSRLRKNGLVSSVRGPGGGYLLGKDAGSIAVGEVISAVDESVDATRCQGKGGCQGGDKCLTHALWRDLSDRLTGFLNNITLGELVNNQEVLDVSGRQHSHDSQRNTRAQDAIDVKLRA

SEQ ID NO: 24 - DNA, 489 bp, Enterobacter timonensis
  source 1..489: mol_type = other DNA
atgcgcctgaccagcaaaggccgctatgcggtgaccgcgatgctggatgtggcgctgaacagcgaagcgggcccggtgccgctggcggatattagcgaacgccagggcattagcctgagctatctggaacagctgtttagccgcctgcgcaaaaacggcctggtgagcagcgtgcgcggcccgggcggcggctatctgctgggcaaagatgcgggcagcattgcggtgggcgaagtgattagcgcggtggatgaaagcgtggatgcgacccgctgccagggcaaaggcggctgccagggcggcgataaatgcctgacccatgcgctgtggcgcgatctgagcgatcgcctgaccggctttctgaacaacattaccctgggcgaactggtgaacaaccaggaagtgctggatgtgagcggccgccagcatagccatgatagccagcgcaacacccgcgcgcaggatgcgattgatgtgaaactgcgcgcg

SEQ ID NO: 25 - protein, 163 aa, Pluralibacter gergoviae
MRLTSKGRYAVTAMLDVALNSESGPVPLADISERQGISLSYLEQLFSRLRKNGLVSSVRGPGGGYLLGKDAGSIAVGEVISAVDESVDATRCQGKAGCQGGDKCLTHALWRDLSDRLTGFLNNITLGELVNNQEVLDVSDRQHIHETQRSTRSQDAIDVKLRA

SEQ ID NO: 26 - DNA, 489 bp, Pluralibacter gergoviae
  source 1..489: mol_type = other DNA
atgcgcctgaccagcaaaggccgctatgcggtgaccgcgatgctggatgtggcgctgaacagcgaaagcggcccggtgccgctggcggatattagcgaacgccagggcattagcctgagctatctggaacagctgtttagccgcctgcgcaaaaacggcctggtgagcagcgtgcgcggcccgggcggcggctatctgctgggcaaagatgcgggcagcattgcggtgggcgaagtgattagcgcggtggatgaaagcgtggatgcgacccgctgccagggcaaagcgggctgccagggcggcgataaatgcctgacccatgcgctgtggcgcgatctgagcgatcgcctgaccggctttctgaacaacattaccctgggcgaactggtgaacaaccaggaagtgctggatgtgagcgatcgccagcatattcatgaaacccagcgcagcacccgcagccaggatgcgattgatgtgaaactgcgcgcg

SEQ ID NO: 27 - protein, 162 aa, Buttiauxella sp
MRLTSKGRYAVTAMLDVALNSESGPVPLADISERQGISLSYLEQLFSRLRKNGLVASVRGPGGGYLLGKEASAIAVGEVISAVDESVDATRCAGKGGCQGGDKCLTHALWRDLSDRLTGFLNNITLGELVNNQEVLDVSGRQHNENHRSTRSQDAIDVKLRA

SEQ ID NO: 28 - DNA, 486 bp, Buttiauxella sp
  source 1..486: mol_type = other DNA
atgcgcctgaccagcaaaggccgctatgcggtgaccgcgatgctggatgtggcgctgaacagcgaaagcggcccggtgccgctggcggatattagcgaacgccagggcattagcctgagctatctggaacagctgtttagccgcctgcgcaaaaacggcctggtggcgagcgtgcgcggcccgggcggcggctatctgctgggcaaagaagcgagcgcgattgcggtgggcgaagtgattagcgcggtggatgaaagcgtggatgcgacccgctgcgcgggcaaaggcggctgccagggcggcgataaatgcctgacccatgcgctgtggcgcgatctgagcgatcgcctgaccggctttctgaacaacattaccctgggcgaactggtgaacaaccaggaagtgctggatgtgagcggccgccagcataacgaaaaccatcgcagcacccgcagccaggatgcgattgatgtgaaactgcgcgcg

SEQ ID NO: 29 - protein, 163 aa, Kosakonia sacchari
MRLTSKGRYAVTAMLDVALNSEAGPVPLADISERQGISLSYLEQLFSRLRKNGLVASVRGPGGGYLLGKDANTIAVGEVISAVDESVDATRCQGKSGCQGGDKCLTHALWRDLSDRLTGFLNNITLGELVNNQEILDVSDRQHNNESHRNTRGQDAIDVKLRA

SEQ ID NO: 30 - DNA, 489 bp, Kosakonia sacchari
  source 1..489: mol_type = other DNA
atgcgcctgaccagcaaaggccgctatgcggtgaccgcgatgctggatgtggcgctgaacagcgaagcgggcccggtgccgctggcggatattagcgaacgccagggcattagcctgagctatctggaacagctgtttagccgcctgcgcaaaaacggcctggtggcgagcgtgcgcggcccgggcggcggctatctgctgggcaaagatgcgaacaccattgcggtgggcgaagtgattagcgcggtggatgaaagcgtggatgcgacccgctgccagggcaaaagcggctgccagggcggcgataaatgcctgacccatgcgctgtggcgcgatctgagcgatcgcctgaccggctttctgaacaacattaccctgggcgaactggtgaacaaccaggaaattctggatgtgagcgatcgccagcataacaacgaaagccatcgcaacacccgcggccaggatgcgattgatgtgaaactgcgcgcg

SEQ ID NO: 31 - protein, 162 aa, Synthetic Construct
MRLTSKGRYAVTAMFDVALNSEAGPVPLADISERQGISLSYLEQLFSRLRKNGLVSSVRGPGGGYLLGKDASSIAVGEVISAVDESVDATRCQGKGGCQGGDKCLTHALWRDLSDRLTGFLNNITLGELVNNQEVLDVSGRQHTHDAPRTRTQDAIDVKLRA

SEQ ID NO: 32 - DNA, 489 bp, Synthetic Construct
  source 1..489: mol_type = other DNA
atgagactgacatctaaagggcgctatgccgtgaccgcaatgtttgacgttgcgctcaactctgaagcgggcccggtaccgttggctgatatttccgaacgtcagggaatttccctttcttatctggaacaactgttttcccgtctgcgtaaaaatggtctggtttccagcgtacgtggaccaggcggtggttatctgttaggcaaagatgccagcagcatcgccgttggcgaagtaattagcgccgttgacgaatctgtagatgccacccgttgtcagggtaaaggcggctgccagggcggcgataaatgcctgacccacgcgctgtggcgtgatttgagcgaccgtctcaccggttttctcaacaacattactttaggcgaactggttaataaccaggaagtgctggatgtgtctggtcgtcagcatactcacgacgcgccacgcacccgcacacaagacgcgatcgacgttaagttacgcgcttaa

SEQ ID NO: 33 - protein, 162 aa, Synthetic Construct
MRLTSKGRYAVTAMLDVALNSEAGPVPLADISERQGISLSYLEQLFSRLRKNGLVSSVRGPGGGYLLGKDASSIAVGEVISAVDESVDATRYQGKGGCQGGDKCLTHALWRDLSDRLTGFLNNITLGELVNNQEVLDVSGRQHTHDAPRTRTQDAIDVKLRA

SEQ ID NO: 34 - DNA, 489 bp, Synthetic Construct
  source 1..489: mol_type = other DNA
atgagactgacatctaaagggcgctatgccgtgaccgcaatgcttgacgttgcgctcaactctgaagcgggcccggtaccgttggctgatatttccgaacgtcagggaatttccctttcttatctggaacaactgttttcccgtctgcgtaaaaatggtctggtttccagcgtacgtggaccaggcggtggttatctgttaggcaaagatgccagcagcatcgccgttggcgaagtaattagcgccgttgacgaatctgtagatgccacccgttatcagggtaaaggcggctgccagggcggcgataaatgcctgacccacgcgctgtggcgtgatttgagcgaccgtctcaccggttttctcaacaacattactttaggcgaactggttaataaccaggaagtgctggatgtgtctggtcgtcagcatactcacgacgcgccacgcacccgcacacaagacgcgatcgacgttaagttacgcgcttaa

SEQ ID NO: 35 - protein, 162 aa, Synthetic Construct
MRLTSKGRYAVTAMLDVALNSEAGPVPLADISERQGISLSYLEQLFSRLRKNGLVSSVRGPGGGYLLGKDASSIAVGEVISAVDESVDATRCQGKGGCQGGDKCLTYALWRDLSDRLTGFLNNITLGELVNNQEVLDVSGRQHTHDAPRTRTQDAIDVKLRA

SEQ ID NO: 36 - DNA, 489 bp, Synthetic Construct
  source 1..489: mol_type = other DNA
atgagactgacatctaaagggcgctatgccgtgaccgcaatgcttgacgttgcgctcaactctgaagcgggcccggtaccgttggctgatatttccgaacgtcagggaatttccctttcttatctggaacaactgttttcccgtctgcgtaaaaatggtctggtttccagcgtacgtggaccaggcggtggttatctgttaggcaaagatgccagcagcatcgccgttggcgaagtaattagcgccgttgacgaatctgtagatgccacccgttgtcagggtaaaggcggctgccagggcggcgataaatgcctgacctacgcgctgtggcgtgatttgagcgaccgtctcaccggttttctcaacaacattactttaggcgaactggttaataaccaggaagtgctggatgtgtctggtcgtcagcatactcacgacgcgccacgcacccgcacacaagacgcgatcgacgttaagttacgcgcttaa

SEQ ID NO: 37 - protein, 346 aa, Escherichia coli
MAHRPRWTLSQVTELFEKPLLDLLFEAQQVHRQHFDPRQVQVSTLLSIKTGACPEDCKYCPQSSRYKTGLEAERLMEVEQVLESARKAKAAGSTRFCMGAAWKNPHERDMPYLEQMVQGVKAMGLEACMTLGTLSESQAQRLANAGLDYYNHNLDTSPEFYGNIITTRTYQERLDTLEKVRDAGIKVCSGGIVGLGETVKDRAGLLLQLANLPTPPESVPINMLVKVKGTPLADNDDVDAFDFIRTIAVARIMMPTSYVRLSAGREQMNEQTQAMCFMAGANSIFYGCKLLTTPNPEEDKDLQLFRKLGLNPQQTAVLAGDNEQQQRLEQALMTPDTDEYYNAAAL

SEQ ID NO: 38 - DNA, 1041 bp, Escherichia coli
  source 1..1041: mol_type = other DNA
atggctcaccgcccacgctggacattgtcgcaagtcacagaattatttgaaaaaccgttgctggatctgctgtttgaagcgcagcaggtgcatcgccagcatttcgatcctcgtcaggtgcaggtcagcacgttgctgtcgattaagaccggagcttgtccggaagattgcaaatactgcccgcaaagctcgcgctacaaaaccgggctggaagccgagcggttgatggaagttgaacaggtgctggagtcggcgcgcaaagcgaaagcggcaggatcgacgcgcttctgtatgggcgcggcgtggaagaatccccacgaacgcgatatgccgtacctggaacaaatggtgcagggggtaaaagcgatggggctggaggcgtgtatgacgctgggcacgttgagtgaatctcaggcgcagcgcctcgcgaacgccgggctggattactacaaccacaacctggacacctcgccggagttttacggcaatatcatcaccacacgcacttatcaggaacgcctcgatacgctggaaaaagtgcgcgatgccgggatcaaagtctgttctggcggcattgtgggcttaggcgaaacggtaaaagatcgcgccggattattgctgcaactggcaaacctgccgacgccgccggaaagcgtgccaatcaacatgctggtgaaggtgaaaggcacgccgcttgccgataacgatgatgtcgatgcctttgattttattcgcaccattgcggtcgcgcggatcatgatgccaacctcttacgtgcgcctttctgccggacgcgagcagatgaacgaacagactcaggcgatgtgctttatggcaggcgcaaactcgattttctacggttgcaaactgctgaccacgccgaatccggaagaagataaagacctgcaactgttccgcaaactggggctaaatccgcagcaaactgccgtgctggcaggggataacgaacaacagcaacgtcttgaacaggcgctgatgaccccggacaccgacgaatattacaacgcggcagcattatga

SEQ ID NO: 39 - protein, 210 aa, Escherichia coli
MVLGKPQTDPTLEWFLSHCHIHKYPSKSTLIHQGEKAETLYYIVKGSVAVLIKDEEGKEMILSYLNQGDFIGELGLFEEGQERSAWVRAKTACEVAEISYKKFRQLIQVNPDILMRLSAQMARRLQVTSEKVGNLAFLDVTGRIAQTLLNLAKQPDAMTHPDGMQIKITRQEIGQIVGCSRETVGRILKMLEDQNLISAHGKTIVVYGTR

SEQ ID NO: 40 - DNA, 633 bp, Escherichia coli
  source 1..633: mol_type = other DNA
atggtgcttggcaaaccgcaaacagacccgactctcgaatggttcttgtctcattgccacattcataagtacccatccaagagcacgcttattcaccagggtgaaaaagcggaaacgctgtactacatcgttaaaggctctgtggcagtgctgatcaaagacgaagagggtaaagaaatgatcctctcctatctgaatcagggtgattttattggcgaactgggcctgtttgaagagggccaggaacgtagcgcatgggtacgtgcgaaaaccgcctgtgaagtggctgaaatttcgtacaaaaaatttcgccaattgattcaggtaaacccggacattctgatgcgtttgtctgcacagatggcgcgtcgtctgcaagtcacttcagagaaagtgggcaacctggcgttcctcgacgtgacgggccgcattgcacagactctgctgaatctggcaaaacaaccagacgctatgactcacccggacggtatgcaaatcaaaattacccgtcaggaaattggtcagattgtcggctgttctcgtgaaaccgtgggacgcattctgaagatgctggaagatcagaacctgatctccgcacacggtaaaaccatcgtcgtttacggcactcgttaa

SEQ ID NO: 41 - protein, 169 aa, Escherichia coli [sequence omitted]

SEQ ID NO: 42 - DNA, 510 bp, Escherichia coli
  source 1..510: mol_type = other DNA
atgggtttgttcgataaactgaaatctctggtttccgacgacaagaaggataccggaactattgagatcattgctccgctctctggcgagatcgtcaatatcgaagacgtgccggatgtcgtttttgcggaaaaaatcgttggtgatggtattgctatcaaaccaacgggtaacaaaatggtcgcgccagtagacggcaccattggtaaaatctttgaaaccaaccacgcattctctatcgaatctgatagcggcgttgaactgttcgtccacttcggtatcgacaccgttgaactgaaaggcgaaggcttcaagcgtattgctgaagaaggtcagcgcgtgaaagttggcgatactgtcattgaatttgatctgccgctgctggaagagaaagccaagtctaccctgactccggttgttatctccaacatggacgaaatcaaagaactgatcaaactgtccggtagcgtaaccgtgggtgaaaccccggttatccgcatcaagaagtaa

SEQ ID NO: 43 - protein, 848 aa, Escherichia coli
MYLYIETLKQRLDAINQLRVDRALAAMGPAFQQVYSLLPTLLHYHHPLMPGYLDGNVPKGICLYTPDETQRHYLNELELYRGMSVQDPPKGELPITGVYTMGSTSSVGQSCSSDLDIWVCHQSWLDSEERQLLQRKCSLLENWAASLGVEVSFFLIDENRFRHNESGSLGGEDCGSTQHILLLDEFYRTAVRLAGKRILWNMVPCDEEEHYDDYVMTLYAQGVLTPNEWLDLGGLSSLSAEEYFGASLWQLYKSIDSPYKAVLKTLLLEAYSWEYPNPRLLAKDIKQRLHDGEIVSFGLDPYCMMLERVTEYLTAIEDFTRLDLVRRCFYLKVCEKLSRERACVGWRRAVLSQLVSEWGWDEARLAMLDNRANWKIDQVREAHNELLDAMMQSYRNLIRFARRNNLSVSASPQDIGVLTRKLYAAFEALPGKVTLVNPQISPDLSEPNLTFIYVPPGRANRSGWYLYNRAPNIESIISHQPLEYNRYLNKLVAWAWFNGLLTSRTRLYIKGNGIVDLPKLQEMVADVSHHFPLRLPAPTPKALYSPCEIRHLAIIVNLEYDPTAAFRNQVVHFDFRKLDVFSFGENQNCLVGSVDLLYRNSWNEVRTLHFNGEQSMIEALKTILGKMHQDAAPPDSVEVFCYSQHLRGLIRTRVQQLVSECIELRLSSTRQETGRFKALRVSGQTWGLFFERLNVSVQKLENAIEFYGAISHNKLHGLSVQVETNHVKLPAVVDGFASEGIIQFFFEETQDENGFNIYILDESNRVEVYHHCEGSKEELVRDVSRFYSSSHDRFTYGSSFINFNLPQFYQIVKVDGREQVIPFRTKSIGNMPPANQDHDTPLLQQYFS

SEQ ID NO: 44 - DNA, 2547 bp, Escherichia coli
  source 1..2547: mol_type = other DNA
ttgtacctctatattgagactctgaaacagagactggatgccataaatcaattgcgtgtggatcgcgcgcttgctgctatggggcctgcattccaacaggtctacagtctactgccgacattgttgcactatcaccatccgctaatgccgggttaccttgatggtaacgttcccaaaggcatttgcctttacacgcctgatgaaactcaacgccactacctgaacgagcttgaactgtatcgtggaatgtcagtacaggatccgccgaaaggtgagcttccaattactggtgtatacaccatgggcagcacctcgtccgtagggcaaagttgttcctctgacctggatatctgggtctgtcatcaatcctggctcgatagcgaagagcgccaattgctacaacgtaaatgtagcctgctggaaaactgggccgcctcgctgggtgtggaagtcagcttcttcctgattgatgaaaaccgcttccgtcataatgaaagcggcagcctggggggcgaagattgtggctccacccagcatatactgctgcttgacgaattttatcgtaccgccgtgcgtctcgccggtaagcgtattctgtggaatatggtgccgtgcgacgaagaagagcattacgacgactatgtgatgacgctttacgcgcagggcgtgctgacgccaaatgaatggctggatctcggtggcttaagctcgctttctgctgaagagtactttggtgccagcctttggcagctctacaagagtatcgattccccatacaaagcggtactgaaaacactgctgctggaagcctattcctgggaatacccgaacccacgtctgctggcgaaagatatcaaacagcgtttgcacgacggcgagattgtatcgtttggtctcgatccatactgcatgatgctggagcgtgttactgaatacctgacggcgattgaagattttacccgtctggatttagtacgtcgctgcttctatttaaaagtgtgcgaaaagctcagccgtgaacgcgcctgcgtaggctggcgtcgcgcagtgttgagccagttagtgagcgagtggggttgggacgaagctcgtctggcaatgctcgataaccgcgctaactggaagattgatcaggtgcgtgaggcgcacaacgagttgctcgacgcgatgatgcagagctaccgtaatctgatccgctttgcgcgtcgcaataaccttagcgtctccgccagtccgcaggatatcggcgtgctgacgcgtaagctgtatgccgcgtttgaagcattaccaggtaaagtgacgctggtaaacccgcagatttcacccgatctctcggaaccgaatctgacctttatttatgtgccgccgggccgggctaaccgttcaggttggtatctgtataaccgcgcgccaaatattgagtcgatcatcagccatcagccgctggaatataaccgttacctgaataaactggtggcgtgggcatggtttaacggcctgctgacctcgcgcacccgtttgtatattaaaggtaacggcattgtcgatttgcctaagttgcaggagatggtcgccgacgtgtcgcaccatttcccgctgcgcttacctgcaccgacaccgaaggcgctctacagcccgtgtgagatccgccatctggcgattatcgttaacctggaatatgacccgacagcggcgttccgcaatcaggtggtgcatttcgatttccgtaagctggatgtcttcagctttggcgagaatcaaaattgcctggtaggtagcgttgacctgctgtaccgcaactcgtggaacgaagtgcgtacgctgcacttcaacggcgagcaatcgatgatcgaagccctgaaaactattctcggcaaaatgcatcaggacgccgcaccgccagatagcgtggaagtcttctgttatagccagcatctgcgcggcttaattcgtactcgcgtgcagcaactggtttctgagtgtattgaattgcgtctttccagcacccgccaggaaaccgggcgtttcaaggcgctgcgcgtttctggtcaaacctgggggttgttcttcgaacgcctgaatgtatcggtacagaaactggaaaacgccatcgagttttatggcgcgatttcgcataacaaactgcacggcctgtcagtgcaggttgaaaccaatcacgtcaaattaccggcggtggtggacggctttgccagcgaagggatcatccagttctttttcgaagaaacgcaagacgagaatggctttaatatctacattctcgacgaaagcaaccgggttgaggtatatcaccactgcgaaggcagcaaagaggagctggtacgtgacgtcagtcgcttctactcgtcatcgcatgaccgttttacctacggctcaagcttcatcaacttcaacctgccgcagttctatcagattgtgaaggttgatggtcgtgaacaggtgattccgttccgcacaaaatctatcggtaacatgccgcctgccaatcaggatcacgatacgccgctattacagcaatatttttcgtga

SEQ ID NO: 45 - protein, 275 aa, Escherichia coli
MESLLTLPLAGEARVRILQITDTHLFAQKHEALLGVNTWESYQAVLEAIRPHQHEFDLIVATGDLAQDQSSAAYQHFAEGIASFRAPCVWLPGNHDFQPAMYSALQDAGISPAKRVFIGEQWQILLLDSQVFGVPHGELSEFQLEWLERKLADAPERHTLLLLHHHPLPAGCSWLDQHSLRNAGELDTVLAKFPHVKYLLCGHIHQELDLDWNGRRLLATPSTCVQFKPHCSNFTLDTIAPGWRTLELHADGTLTTEVHRLADTRFQPDTASEGY

SEQ ID NO: 46 - DNA, 828 bp, Escherichia coli
  source 1..828: mol_type = other DNA
ttggaaagcctgttaacccttcctctggctggtgaggccagagtcaggattttacaaattaccgacactcacctgtttgcacaaaagcacgaagccctgttaggggtaaacacctgggagagttaccaggcggtgctggaggcgattcggccacaccagcacgaattcgacctgattgtcgcgacaggtgatttagcgcaggatcaatcctctgcggcctatcagcatttcgctgaaggcatcgcaagttttcgtgcgccctgcgtctggctgccgggcaaccacgatttccagcccgcgatgtacagcgcgttacaggatgcgggtatctccccggcgaagcgcgtgtttattggtgagcaatggcaaatcctgttgctggatagccaggtgtttggcgtgccgcacggtgagctgagcgagtttcagcttgagtggctggaacgtaaactggccgatgcgccagaacgccatacgttgctgctgctgcatcatcatccgctacctgcgggttgtagttggctcgatcaacacagtctgcgtaacgcgggcgaactggataccgtgctggcgaagtttccgcacgtcaaatacttgctgtgcggtcatattcatcaggagctggatctcgactggaatggtcgccgcctgctggcaacgccgtcgacctgtgtgcagtttaagccgcactgttccaactttacgctggataccatcgcgcccggctggcgtactctcgagttacatgctgatggcacgctgaccaccgaggtgcatcgcctggcggacacacgtttccaacctgataccgcttcagaaggctactga

SEQ ID NO: 47 - protein, 442 aa, Leptospira interrogans
MALTFQEILDRIRIIDRDVTELNRLKSRLPADRPYSSSLQISFDKQINELLNERVGLMELEVLDPPSWILGVPTTGISQETPVPLKGLFPSGDLSKEKPDDQDVINFLRELPKTEIHLHLEACVNKDTMKRLMAKNGINVTDEEFEAKFNFKDLNSFIQVFFFIQSLVKEPSDFSFFIESLAEYMRANNILYTEVFFAPSKFIQNGLDFEEMIDFLVNRIREEKENDGIVIRLLVDVSRSFGPENAMKNLDRVLKLRHPEVIGIGLGGAELMGPARDYQGVFQKAREAGLRVVAHSGEDDGPWAIWEAVELLKAERIGHGTSAIQDPELVKYLRENHIPIEICVTSNVFTGKYVRKEQNHPVRYYYDQGLPLSINTDDPEIFNVNLTYEYYKLWRFLDFSLDEIVDLIRQGVFASFHPNKESLWAEMEKNIHLVKTRYGLKR

SEQ ID NO: 48 - DNA, 1329 bp, Leptospira interrogans
  source 1..1329: mol_type = other DNA
gtggctttaacttttcaagagattttagatagaattcggatcatcgatcgagacgtgaccgaactgaaccgtttgaagtctagacttcctgcggaccgaccttattcttcttctttacaaatttcttttgataaacaaatcaacgaactcttaaacgaaagagtcggtctcatggaacttgaggtcctagatccgccttcttggatcttaggagttcctacaactgggatttctcaggaaactccggttccactcaaaggcctttttccgtccggagatctttctaaggaaaaaccggacgatcaagacgtaattaatttcctacgagaacttcctaaaacggaaattcatctccaccttgaggcgtgtgttaataaggacacgatgaaaagattgatggctaaaaatggaattaatgtaacggatgaagagtttgaagctaaattcaattttaaggatttaaacagtttcattcaggtattcttttttattcagtcgcttgttaaagaaccttctgatttctcattttttatagaaagtctcgcggaatacatgagagcgaataacatcctttatacggaagttttttttgcaccttctaagtttattcagaacggtctcgattttgaagaaatgatcgattttttagtcaatcgaattcgagaagaaaaagaaaacgacggtatcgtcatacgtctattagttgatgtttccagatcctttgggccagaaaatgcgatgaaaaatctagatcgggttctaaaactgagacatccggaagtgattggaattggtcttggaggcgcggaacttatgggacccgccagggattatcaaggggtttttcaaaaagcgagggaagcggggctcagagtggtggcgcattccggagaagacgatggtccttgggcaatttgggaggcggtggaacttctcaaagcggagaggattggacacggaacttctgcgatccaagatcccgagttagtaaaatatcttagggagaatcatattccaatcgagatttgtgtgacgtctaacgtatttactggtaaatacgttagaaaagaacaaaaccatccagttcgttattattacgatcagggtcttcctcttagtatcaatacggacgatcctgagatattcaatgtaaatcttacatacgaatattataaactttggagattcttagatttctctttggatgagatcgtagatttgatccgtcaaggtgtttttgcttccttccatcccaataaagaatctctctgggccgaaatggaaaaaaacattcatctcgtaaaaacaagatacggtctaaaaagataa

SEQ ID NO: 49 - DNA, 97 bp, Synthetic Construct
  source 1..97: mol_type = other DNA
tcataaaaaatttatttgctttgtgagcggataacaattataatagattcaaatcggaggttctctaactagtatctctagagctaaggaggtaaat

SEQ ID NO: 50 - DNA, 36 bp, Synthetic Construct
  source 1..36: mol_type = other DNA
ttgacaattaatcatccggctcgtaatgtttgtgga

SEQ ID NO: 51 - protein, 631 aa, Escherichia coli
MSATKLTRREQRARAQHFIDTLEGTAFPNSKRIYITGTHPGVRVPMREIQLSPTLIGGSKEQPQYEENEAIPVYDTSGPYGDPQIAINVQQGLAKLRQPWIDARGDTEELTVRSSDYTKARLADDGLDELRFSGVLTPKRAKAGRRVTQLHYARQGIITPEMEFIAIRENMGRERIRSEVLRHQHPGMSFGAHLPENITAEFVRDEVAAGRAIIPANINHPESEPMIIGRNFLVKVNANIGNSAVTSSIEEEVEKLVWSTRWGADTVMDLSTGRYIHETREWILRNSPVPIGTVPIYQALEKVNGIAEDLTWEAFRDTLLEQAEQGVDYFTIHAGVLLRYVPMTAKRLTGIVSRGGSIMAKWCLSHHQENFLYQHFREICEICAAYDVSLSLGDGLRPGSIQDANDEAQFAELHTLGELTKIAWEYDVQVMIEGPGHVPMQMIRRNMTEELEHCHEAPFYTLGPLTTDIAPGYDHFTSGIGAAMIGWFGCAMLCYVTPKEHLGLPNKEDVKQGLITYKIAAHAADLAKGHPGAQIRDNAMSKARFEFRWEDQFNLALDPFTARAYHDETLPQESGKVAHFCSMCGPKFCSMKISQEVRDYAATQTIEMGMADMSENFRARGGEIYLRKEEA

SEQ ID NO: 52 - DNA, 1884 bp, Escherichia coli
  source 1..1884: mol_type = other DNA
atgtctgcaacaaaactgacccgccgcgaacaacgcgcccgggcccaacattttatcgacaccctggaaggcaccgcctttcccaactcaaaacgcatttatatcactggcacacaccccggcgtgcgcgtgccgatgcgtgagatccagcttagcccgacgctaattggcggtagcaaagaacagccgcagtacgaagaaaacgaagcgattccggtctacgacacctccggcccgtatggtgatccgcagattgccattaacgtgcagcaagggctggcaaaactacgccagccgtggatcgatgcgcgcggcgataccgaagaacttaccgtgcgcagttccgattacactaaagcgcggctggcagatgatggcctcgacgaactgcgttttagcggcgtactaacaccaaaacgcgccaaagcaggacgccgtgtcacccaactgcactacgcccgccagggcatcatcacgccggaaatggaattcatcgccatccgcgagaatatgggccgcgagcgcatccgtagcgaggttttacgccaccagcatccgggaatgagctttggcgcacatctgccggaaaatatcactgcggaatttgtccgtgatgaagttgctgccggacgtgcgattatcccggccaacattaatcatccggaatcggagccgatgattattggtcgcaatttcctggtaaaagttaacgccaatatcggcaactcggcggtcacctcttccatcgaagaagaagtggaaaagctggtatggtccacgcgctggggagcggatacggtgatggatctctccaccggtcgctatattcacgaaacccgcgagtggattttgcgtaacagcccggtgccgatcggtacagtgccgatctaccaggcgctggagaaggttaacgggatcgccgaagatcttacctgggaagcgttccgcgacacgctgctggaacaggccgagcaaggtgtggattacttcactatccatgcgggcgtactgctgcgctatgtgccgatgaccgcgaaacgcctgaccggtatcgtctctcgcggcggttcgattatggcgaaatggtgcctctcccatcatcaggaaaatttcctctatcaacacttccgcgaaatttgtgaaatctgtgccgcttatgacgtttcgctgtcgctgggcgacggtctgcgccccggttctattcaggacgccaacgatgaagcgcagtttgccgagctgcatacgctgggcgaactgaccaaaattgcctgggaatatgacgtgcaggtgatgattgaaggcccaggccacgtgccgatgcagatgatccgccgcaatatgaccgaggagttagagcactgccacgaagcgccgttttacactctggggccgctaactaccgatattgcgccgggctatgaccacttcacgtcggggattggtgcggcgatgattggctggtttggctgcgcgatgctctgttacgtaacgccaaaagagcatctgggtctgcccaataaagaagatgttaagcaggggcttatcacctataagattgctgcccacgccgctgacctggcgaaagggcatccgggcgcgcaaattcgcgataacgccatgtcgaaagcccgcttcgaatttcgctgggaagaccagtttaatctggccctcgacccgtttaccgcccgcgcttatcacgatgaaaccctgccgcaagagtcaggtaaagtcgcccatttttgctccatgtgtgggccgaaattctgctcgatgaaaatcagccaggaagtgcgtgattacgccgccacgcaaactattgaaatgggaatggcggatatgtcggagaacttccgtgccagaggcgggagaaatctacctgcgtaa

SEQ ID NO: 53 - protein, 266 aa, Escherichia coli
MKRINALTIAGTDPSGGAGIQADLKTFSALGAYGCSVITALVAQNTRGVQSVYRIEPDFVAAQLDSVFSDVRIDTTKIGMLAETDIVEAVAERLQRYQIQNVVLDTVMLAKSGDPLLSPSAVATLRSRLLPQVSLITPNLPEAAALLDAPHARTEQEMLEQGRSLLAMGCGAVLMKGGHLDDEQSPDWLFTREGEQRFTAPRIMTKNTHGTGCTLSAALAALRPRHTNWADTVQEAKSWLSSALAQADTLEVGHGIGPVHHFHAWW

SEQ ID NO: 54 - DNA, 801 bp, Escherichia coli
  source 1..801: mol_type = other DNA
atgaaacgaattaacgctctgacgattgccggtactgatccgagtggtggtgcggggattcaggccgatcttaaaaccttctcggcacttggcgcttatggttgctcagttattactgcactggtggcgcaaaatacccgtggcgtacagtcggtgtatcgcattgagcctgattttgtcgccgcccagctcgattcggtgttcagcgatgtgcgaatcgataccactaaaatcggtatgctggcggaaaccgatattgttgaagcggtggcagaacggttgcaacgttatcagatccaaaacgtggtactcgacaccgttatgctggcaaaaagcggcgacccgctgctttcaccttcggcggttgctacgctgcgcagtcgattattgccacaggtttcattaataacgccaaacttgcccgaagctgccgccttgctcgacgcgccacacgcgcgcaccgaacaggaaatgctggaacaagggcgatcgctgttggcgatgggctgtggcgcagtgctaatgaaaggtggtcatctggatgatgagcaaagcccggactggctgtttacccgcgagggtgaacaacggtttaccgcaccgcgcattatgaccaaaaacacccacggcactggttgtacactctctgcggcgttggctgcactacgcccgcgccatacaaactgggctgacaccgtacaggaggcaaaaagctggctttcatcggcgttagcccaggccgacacgctggaagttggtcacggtattggtccggttcaccacttccacgcctggtggtga

SEQ ID NO: 55 - protein, 251 aa, Escherichia coli
MNDRDFMRYSRQILLDDIALDGQQKLLDSQVLIIGLGGLGTPAALYLAGAGVGTLVLADDDDVHLSNLQRQILFTTEDIDRPKSQVSQQRLTQLNPDIQLTALQQRLTGEALKDAVARADVVLDCTDNMATRQEINAACVALNTPLITASAVGFGGQLMVLTPPWEQGCYRCLWPDNQEPERNCRTAGVVGPVVGVMGTLQALEAIKLLSGIETPAGELRLFDGKSSQWRSLALRRASGCPVCGGSNADPV

SEQ ID NO: 56 - DNA, 753 bp, Escherichia coli
  source 1..753: mol_type = other DNA
atgaatgaccgtgactttatgcgttatagccgccaaatcctgctcgacgatatcgctctggacgggcagcaaaaactgctcgacagccaggtgctgattatcggtctgggcgggctgggtacacctgctgcgctgtacctggcgggcgctggcgtcgggacgctggtactggcagatgacgacgatgtgcatttaagcaatctgcaacgacaaatcctctttaccactgaagatatcgatcgcccgaaatcgcaggtcagccaacagcgactgacacagttgaatcccgacattcaactgacagcattacaacaacggttaacgggtgaggcgttaaaagatgcggttgcacgggccgatgtggtgctcgactgtaccgacaatatggcgactcgccaggagattaatgccgcctgcgtggcactcaacacgccgcttatcaccgccagcgcggtcggatttggcggtcagttgatggtactgacgccgccctgggagcaggggtgttaccgctgcctgtggccagataaccaggagccagaacgcaactgccgcacggcgggcgtggttggcccggtggtcggggttatgggcactttgcaggcactggaagccattaagttattaagcggtatagagacacctgcgggagaactccgactgttcgacggtaaatcgagccagtgcagcctggcgttgcgccgcgccagtggttgcccggtatgcggaggaagcaatgcagatcctgtttaa

SEQ ID NO: 57 - protein, 66 aa, Escherichia coli
MQILFNDQAMQCAAGQTVHELLEQLDQRQAGAALAINQQIVPREQWAQHIVQDGDQILLFQVIAGG

SEQ ID NO: 58 - DNA, 201 bp, Escherichia coli
  source 1..201: mol_type = other DNA
atgcagatcctgtttaacgatcaagcgatgcagtgcgccgccgggcaaactgttcacgaactactggagcaactcgaccaacgacaagcgggcgcggccctggcgattaatcagcaaatcgtcccgcgtgagcagtgggcgcaacatatcgtgcaggatggcgaccagatcctgctttttcaggttattgcagggggttga

SEQ ID NO: 59 - protein, 377 aa, Escherichia coli
MKTFSDRWRQLDWDDIRLRINGKTAADVERALNASQLTRDDMMALLSPAASGYLEQLAQRAQRLTRQRFGNTVSFYVPLYLSNLCANDCTYCGFSMSNRIKRKTLDEADIARESAAIREMGFEHLLLVTGEHQAKVGMDYFRRHLPALREQFSSLQMEVQPLAETEYAELKQLGLDGVMVYQETYHEATYARHHLKGKKQDFFWRLETPDRLGRAGIDKIGLGALIGLSDNWRVDSYMVAEHLLWLQQHYWQSRYSVSFPRLRPCTGGIEPASIMDERQLVQTICAFRLLAPEIELSLSTRESPWFRDRVIPLAINNVSAFSKTQPGGYADNHPELEQFSPHDDRRPEAVAAALTAQGLQPVWKDWDSYLGRASQRL

SEQ ID NO: 60 - DNA, 1134 bp, Escherichia coli
  source 1..1134: mol_type = other DNA
atgaaaaccttcagcgatcgctggcgacaactggactgggacgacatccgcctgcgtatcaacggcaaaacggctgctgacgtagagcgggcgctaaatgcctcgcaactcacccgcgacgacatgatggcgctgttatcgcctgccgccagtggctatctggaacaactggcccaacgggcgcagcgtctgacccgtcagcgatttggcaacacagttagtttctacgtcccgctttatctttccaatctttgcgctaacgactgcacgtactgtggattttccatgagtaatcgcatcaagcgcaaaacgctggatgaagcggatattgccagggaaagtgccgctatacgggagatgggctttgaacatctgctgttagtcactggtgaacatcaggcgaaagtggggatggattactttcgtcgtcatctccctgcccttcgtgaacagttctcttcactacagatggaagtgcaaccgctggcggagacggaatacgccgagttaaagcaacttggtctggatggcgtgatggtttatcaggagacatatcacgaggcgacttatgcccgccatcatctgaaaggcaaaaaacaggacttcttctggcggctggaaaccccggatcggctggggcgtgcggggattgataagataggcctcggcgcgctaattggcctttccgacaactggcgcgttgacagctatatggttgccgaacatttgctatggctgcaacagcattactggcaaagccgttactctgtctcctttccgcgcctgcgcccgtgtactggcggcattgagcctgcgtcgattatggatgaacgccagttagtgcaaaccatctgcgccttccgactgcttgcaccggagattgaactgtcactctccacgcgggaatcaccgtggtttcgcgatcgcgttattccgctggcgatcaataacgtcagcgccttctcgaaaacgcagccaggtggctatgccgataatcaccccgagttggaacagttctcaccgcacgacgatcgcagaccggaagcggttgctgccgcgttaaccgctcagggtttgcagccggtatggaaagactgggacagctatctgggacgcgcctcgcaaagactatga

SEQ ID NO: 61 - protein, 256 aa, Escherichia coli
MLRIADKTFDSHLFTGTGKFASSQLMVEAIRASGSQLVTLAMKRVDLRQHNDAILEPLIAAGVTLLPNTSGAKTAEEAIFAAHLAREALGTNWLKLEIHPDARWLLPDPIETLKAAETLVQQGFVVLPYCGADPVLCKRLEEVGCAAVMPLGAPIGSNQGLETRAMLEIIIQQATVPVVVDAGIGVPSHAAQALEMGADAVLVNTAIAVADDPVNMAKAFRLAVEAGLLARQSGPGSRSYFAHATSPLTGFLEASA

SEQ ID NO: 62 - DNA, 771 bp, Escherichia coli
  source 1..771: mol_type = other DNA
atgttacgtattgcggacaaaacgtttgattcacatctgtttaccggcacagggaaattcgcttcttcacaactgatggtggaggcgatccgcgcttccggcagccagctggtgacactggcgatgaaacgtgtcgacttgcgccagcacaacgacgctatcctcgaaccgcttatcgcggcgggtgtgaccctgctgccaaatacatccggggcgaaaacagcggaagaagccattttcgccgcccatctggctcgtgaagcgttaggcacaaactggttaaaattagagattcaccctgacgcccgctggctgttgcccgatcccatcgaaaccctgaaagccgccgaaacgctggtacaacagggatttgtcgtgctgccttactgcggggccgatccggtattgtgtaaacgtctggaagaagtcggctgtgcagcggtgatgccgctcggcgcgccgattggctcgaatcagggactggaaacccgcgccatgctggagattattatccagcaggccacagtgccggtggttgtcgatgctggcatcggcgttcccagccatgccgcgcaggcgctggaaatgggggccgacgcggtgttagtgaatacggcgattgccgtcgcggacgatcccgtcaacatggcgaaggcatttcgtctggcggtagaagcaggcctactggcacgtcagtccggaccgggcagccgcagttattttgctcatgccaccagcccgctgaccggatttctggaggcatcggcatga

SEQ ID NO: 63 - protein, 262 aa, Escherichia coli
MQVDLLGSAQSAHALHLFHQHSPLVHCMTNDVVQTFTANTLLALGASPAMVIETEEASQFAAIASALLINVGTLTQPRAQAMRAAVEQAKSSQTPWTLDPVAVGALDYRRHFCHELLSFKPAAIRGNASEIMALAGIANGGRGVDTTDAAANAIPAAQTLARETGAIVVVTGEMDYVTDGHRIIGIHGGDPLMTKVVGTGCALSAVVAACCALPGDTLENVASACHWMKQAGERAVARSEGPGSFVPHFLDALWQLTQEVQA

SEQ ID NO: 64 - DNA, 789 bp, Escherichia coli
  source 1..789: mol_type = other DNA
atgcaagtcgacctgctgggttcagcgcaatctgcgcacgcgttacacctttttcaccaacattcccctcttgtgcactgcatgaccaatgatgtggtgcaaacctttaccgccaataccttgctggcgctcggtgcatcgccagcgatggttatcgaaaccgaagaggccagtcagtttgcggctatcgccagtgccttgttgattaacgttggcacactgacgcagccacgcgctcaggcgatgcgtgctgccgttgagcaagcaaaaagctctcaaacaccctggacgcttgatccagtagcggtgggtgcgctcgattatcgccgccatttttgtcatgaacttttatcttttaaaccggcagcgatacgtggtaatgcttcggaaatcatggcattagctggcattgctaatggcggacggggagtggataccactgacgccgcagctaacgcgatacccgctgcacaaacactggcacgggaaactggcgcaatcgtcgtggtcactggcgagatggattatgttaccgatggacatcgtatcattggtattcacggtggtgatccgttaatgaccaaagtggtaggaactggctgtgcattatcggcggttgtcgctgcctgctgtgcgttaccaggcgatacgctggaaaatgtcgcatctgcctgtcactggatgaaacaagccggagaacgcgcagtcgccagaagcgaggggccaggcagttttgttccacatttccttgatgcgctctggcaattgacgcaggaggtgcaggcatga

SEQ ID NO: 65 - protein, 617 aa, Arabidopsis thaliana
MRFLFPTRLINNSSLGLLRSPHTTAPIRSLWFRTKSPVFRSATTPIMTAVAFSSSLSIPPTSEEALPGKLWIKFNRECLFSIYSPFAVCLAAGNLKIDTFRQYIAQDVHFLKAFAHAYELAADCADDDDDKLAISDLRKSVMEELKMHDSFVQDWDLDINKEVSVNSATLRYTEFLLATASGKVEGCKAPGMLDTPFEKTKVAAYTLGAVTPCMRLYAFLGKEFGSLLDLSDVNHPYKKWIDNYSSDAFQASAKQTEDLLEKLSVSMTGEELDIIEKLYQQAMKLEVEFFHAQPLAQPTIVPLLKNHSKDDLVIFSDFDLTCTVVDSSAILAEIAIVTAPKDEQSRSGQQIHRMLSSDLKNTWNLLSKQYTEHYEECIESILNKKKADKFDYEGLCKALEQLSDFEKEANNRVIESGVLKGLNLEDIKRAGERLILQDGCINVFQKILKTENLNAELHVLSYCWCGDLIRAAFSAGGVDAVEVHANEFTFEESISTGEIERKVESPINKAQQFKSILQNRKNENNKKSFLSVYIGDSVGDLLCLLEADIGIVVSSSSSLRRVGSHFGVSFVPLFSGIVQKQKQHTEESSSSAWKGLSGTLYTVSSWAEIHSFALGWE

SEQ ID NO: 66 - DNA, 801 bp, Arabidopsis thaliana; identical to SEQ ID NO: 54
  source 1..801: mol_type = other DNA

SEQ ID NO: 67 - protein, 274 aa, Escherichia coli
MPFRSNNPITRDELLSRFFPQYHPVTTFNSGLSGGSFLIEHQGQRFVVRQPHDPDAPQSAFLRQYRALSQLPACIAPKPHLYLRDWMVVDYLPGAVKTYLPDTNELAGLLYYLHQQPRFGWRITLLPLLELYWQQSDPARRTVGWLRMLKRLRKAREPRPLRLSPLHMDVHAGNLVHSASGLKLIDWEYAGDGDIALELAAVWVENTEQHRQLVNDYATRAKIYPAQLWRQVRRWFPWLLMLKAGWFEYRWRQTGDQQFIRLADDTWRQLLIKQ

SEQ ID NO: 68 - DNA, 825 bp, Escherichia coli
  source 1..825: mol_type = other DNA
gtgccgtttcgcagcaataatcccatcacgcgcgacgaattgctgtcgcgctttttcccgcagtatcatcccgtcacgacgtttaatagtgggcttagtggcgggagttttctcattgaacatcagggccagcgttttgttgtgcgtcagccgcacgatcctgatgcgccgcagtccgcgttcttgcgccagtatcgggctttatcacaactacccgcatgcattgcaccgaagccgcatttatatctccgtgactggatggtagtcgactatctgcccggcgcggtaaaaacgtatttgccggataccaacgaactggcaggcttgctgtattatctacatcaacaaccacgttttggctggcgaataacgctgttgccgttactggaactgtactggcagcaaagcgatccggcgcggcggacagtgggttggctgcgaatgttaaaacgtctgcgcaaagcgcgggaaccacggcctttacgcttaagtccattgcatatggatgtccacgccggaaatttagtgcatagcgcgtcagggttaaaactcatcgactgggagtatgccggagatggtgatatcgcgctggaactggcggcggtgtgggtggaaaatactgaacagcaccggcaattggtcaatgactatgccactcgcgcgaagatttatccggcgcaattatggcgtcaggtcaggcgatggtttccctggctgctgatgctcaaagcagggtggtttgagtaccgctggcgacaaaccggcgatcaacaatttatcaggctggccgatgacacctggcggcagctattaataaaacaataa

SEQ ID NO: 69 - protein, 325 aa, Escherichia coli
MACGEFSLIARYFDRVRSSRLDVELGIGDDCALLNIPEKQTLAISTDTLVAGNHFLPDIDPADLAYKALAVNLSDLAAMGADPAWLTLALTLPDVDEAWLESFSDSLFDLLNYYDMQLIGGDTTRGPLSMTLGIHGFVPMGRALTRSGAKPGDWIYVTGTPGDSAAGLAILQNRLQVADAKDADYLIKRHLRPSPRILQGQALRDLANSAIDLSDGLISDLGHIVKASDCGARIDLALLPFSDALSRHVEPEQALRWALSGGEDYELCFTVPELNRGALDVALGHLGVPFTCIGQMTADIEGLCFIRDGEPVTLDWKGYDHFATP

SEQ ID NO: 70 - DNA, 978 bp, Escherichia coli
  source 1..978: mol_type = other DNA
atggcatgtggcgagttctccctgattgcccgttattttgaccgtgtaagaagttctcgtcttgatgtcgaactgggcatcggcgacgattgcgcacttctcaatatccccgagaaacagaccctggcgatcagcactgatacgctggtggcgggtaaccatttcctccctgatatcgatcctgctgatctggcttataaagcactggcggtgaacctaagcgatctggcagcgatgggggccgatccggcctggctgacgctggcattaaccttaccggacgtagacgaagcgtggcttgagtccttcagcgacagtttgtttgatcttctcaattattacgatatgcaactcattggcggcgataccacgcgtgggccattatcaatgacgttgggtatccacggctttgttccgatgggacgagccttaacgcgctctggggcgaaaccgggtgactggatctatgtgaccggtacaccgggcgatagcgccgccgggctggcgattttgcaaaaccgtttgcaggttgccgatgctaaagatgcggactacttgatcaaacgtcatctccgtccatcgccgcgtattttacaggggcaggcactgcgcgatctggcaaattcagccatcgatctctctgacggtttgatttccgatctcgggcatatcgtgaaagccagcgactgcggcgcacgtattgacctggcattgctgccgttttctgatgcgctttctcgccatgttgaaccggaacaggagctgcgctgggcgctctctggcggtgaagattacgagttgtgtttcactgtgccggaactgaaccgtggcgcgctggatgtggctctcggacacctgggcgtaccgtttacctgtatcgggcaaatgaccgccgatatcgaagggctttgttttattcgtgacggcgaacctgttacattagactggaaaggatatgaccattttgccacgccataa

SEQ ID NO: 71 - protein, 211 aa, Escherichia coli
MYQPDFPPVPFRSGLYPVVDSVQWIERLLDAGVRTLQLRIKDRRDEEVEADVVAAIALGRRYNARLFINDYWRLAIKHQAYGVHLGQEDLQATDLNAIRAAGLRLGVSTHDDMEIDVALAARPSYIALGHVFPTQTKQMPSAPQGLEQLARHVERLADYPTVAIGGISLARAPAVIATGVGSIAVVSAITQAADWRLATAQLLEIAGVGDE

SEQ ID NO: 72 - DNA, 636 bp, Escherichia coli
  source 1..636: mol_type = other DNA
atgtatcagcctgattttcctcctgtaccttttcgttcaggactgtacccggtggtggacagcgtacagtggatcgaacgtctgttggatgcaggcgtacgtactctccagctacgcatcaaagatcggcgcgatgaagaggtggaagccgatgtcgtggcggcaattgcgctgggccgccgctataacgcgcgattgtttatcaacgattactggcggctggcgatcaagcatcaggcgtatggcgtccatttggggcaggaagatttgcaagccaccgatctcaatgccatccgcgcggcaggcctgcggctgggcgtttcgacacatgacgatatggaaatcgacgtcgcgctggcagcacgcccctcttatatcgcgctgggacatgtgttcccgacgcaaaccaaacagatgccttctgcaccgcaggggctggaacagctggcacggcatgttgagcgactggcggattatcccaccgtggcgattggcggtatcagtctggcacgcgcgcctgcggtgatagcaacgggtgtcggcagtatcgccgtcgtcagcgccattactcaagccgcagactggcgtttggcaacggcacagttgctggaaattgcaggagttggcgatgaatga

SEQ ID NO: 73 - protein, 482 aa, Escherichia coli
MKFIIKLFPEITIKSQSVRLRFIKILTGNIRNVLKHYDETLAVVRHWDNIEVRAKDENQRLAIRDALTRIPGIHHILEVEDVPFTDMHDIFEKALVQYRDQLEGKTFCVRVKRRGKHDFSSIDVERYVGGGLNQHIESARVKLTNPDVTVHLEVEDDRLLLIKGRYEGIGGFPIGTQEDVLSLISGGFDSGVSSYMLMRRGCRVHYCFFNLGGAAHEIGVRQVAHYLWNRFGSSHRVRFVAINFEPVVGEILEKIDDGQMGVILKRMMVRAASKVAERYGVQALVTGEALGQVSSQTLTNLRLIDNVSDTLILRPLISYDKEHIINLARQIGTEDFARTMPEYCGVISKSPTVKAVKSKIEAEEEKFDFSILDKVVEEANNVDIREIAQQTEQEVVEVETVNGFGPNDVILDIRSIDEQEDKPLKVEGIDVVSLPFYKLSTKFGDLDQNKTWLLWCERGVMSRLQALYLREQGFNNVKVYRP

SEQ ID NO: 74 - DNA, 1449 bp, Escherichia coli
  source 1..1449: mol_type = other DNA
atgaagtttatcattaaattgttcccggaaatcaccatcaaaagccaatctgtgcgcttgcgctttataaaaatccttaccgggaacattcgtaacgttttaaagcactatgatgagacgctcgctgtcgtccgccactgggataacatcgaagttcgcgcaaaagatgaaaaccagcgtctggctattcgcgacgctctgacccgtattccgggtatccaccatattctcgaagtcgaagacgtgccgtttaccgacatgcacgatattttcgagaaagcgttggttcagtatcgcgatcagctggaaggcaaaaccttctgcgtacgcgtgaagcgccgtggcaaacatgattttagctcgattgatgtggaacgttacgtcggcggcggtttaaatcagcatattgaatccgcgcgcgtgaagctgaccaatccggatgtgactgtccatctggaagtggaagacgatcgtctcctgctgattaaaggccgctacgaaggtattggcggtttcccgatcggcacccaggaagatgtgctgtcgctcatttccggtggtttcgactccggtgtttccagttatatgttgatgcgtcgcggctgccgcgtgcattactgcttctttaacctcggcggcgcggcgcatgaaattggcgtgcgtcaggtggcgcattatctgtggaaccgctttggcagctcccaccgcgtgcgttttgtcgctattaatttcgaaccggtcgtcggggaaattctcgagaaaatcgacgacggtcagatgggcgttatcctcaaacgtatgatggtgcgtgccgcatctaaagtggctgaacgttacggcgtacaggcgctggtcaccggcgaagcgctcggccaggtgtccagccagacgctgaccaacctgcgcctgattgataacgtctccgacacgctgatcctgcgtccgctgatctcttacgacaaagagcacatcatcaacctggcccgccagattggcaccgaagactttgctcgcacgatgccggaatattgtggtgtgatctccaaaagcccgacggtgaaagcagttaaatcgaagattgaagcggaagaagagaagttcgacttcagcattctcgataaagtggttgaggaagcgaataacgttgatatccgcgaaatcgcccagcagaccgagcaggaagtggtggaagtggaaaccgtcaatggcttcggcccgaacgacgtgatcctcgatatccgttctatcgatgaacaggaagataagccactgaaagtcgaagggattgatgtggtttctctgccgttctataaactgagcaccaaatttggcgatctcgaccagaacaaaacctggctgctgtggtgtgagcgcggggtgatgagccgtctgcaggcgctctatctgcgcgagcagggctttaacaatgtgaaggtatatcgcccgtaa

SEQ ID NO: 75 - protein, 404 aa, Escherichia coli
MKLPIYLDYSATTPVDPRVAEKMMQFMTMDGTFGNPASRSHRFGWQAEEAVDIARNQIADLVGADPREIVFTSGATESDNLAIKGAANFYQKKGKHIITSKTEHKAVLDTCRQLEREGFEVTYLAPQRNGIIDLKELEAAMRDDTILVSIMHVNNEIGVVQDIAAIGEMCRARGIIYHVDATQSVGKLPIDLSQLKVDLMSFSGHKIYGPKGIGALYVRRKPRVRIEAQMHGGGHERGMRSGTLPVHQIVGMGEAYRIAKEEMATEMERLRGLRNRLWNGIKDIEEVYLNGDLEHGAPNILNVSFNYVEGESLIMALKDLAVSSGSACTSASLEPSYVLRALGLNDELAHSSIRFSLGRFTTEEEIDYTIELVRKSIGRLRDLSPLWEMYKQGVDLNSIEWAHH

SEQ ID NO: 76 - DNA, 1215 bp, Escherichia coli
  source 1..1215: mol_type = other DNA
atgaaattaccgatttatctcgactactccgcaaccacgccggtggacccgcgtgttgccgagaaaatgatgcagtttatgacgatggacggaacctttggtaacccggcctcccgttctcaccgtttcggctggcaggctgaagaagcggtagatatcgcccgtaatcagattgccgatctggtcggcgctgatccgcgtgaaatcgtctttacctctggtgcaaccgaatctgacaacctggcgatcaaaggtgcagccaacttttatcagaaaaaaggcaagcacatcatcaccagcaaaaccgaacacaaagcggtactggatacctgccgtcagctggagcgcgaaggttttgaagtcacctacctggcaccgcagcgtaacggcattatcgacctgaaagaacttgaagcagcgatgcgtgacgacaccatcctcgtgtccatcatgcacgtaaataacgaaatcggcgtggtgcaggatatcgcggctatcggcgaaatgtgccgtgctcgtggcattatctatcacgttgatgcaacccagagcgtgggtaaactgcctatcgacctgagccagttgaaagttgacctgatgtctttctccggtcacaaaatctatggcccgaaaggtatcggtgcgctgtatgtacgtcgtaaaccgcgcgtacgcatcgaagcgcaaatgcacggcggcggtcacgagcgcggtatgcgttccggcactctgcctgttcaccagatcgtcggaatgggcgaggcctatcgcatcgcaaaagaagagatggcgaccgagatggaacgtctgcgcggcctgcgtaaccgtctgtggaacggcatcaaagatatcgaagaagtttacctgaacggtgacctggaacacggtgcgccgaacattctcaacgtcagcttcaactacgttgaaggtgagtcgctgattatggcgctgaaagacctcgcagtttcttcaggttccgcctgtacgtcagcaagcctcgaaccgtcctacgtgctgcgcgcgctggggctgaacgacgagctggcacatagctctatccgtttctctttaggtcgttttactactgaagaagagatcgactacaccatcgagttagttcgtaaatccatcggtcgtctgcgtgacctttctccgctgtgggaaatgtacaagcagggcgtggatctgaacagcatcgaatgggctcatcattaa

SEQ ID NO: 77 - protein, 162 aa, Escherichia coli; identical to SEQ ID NO: 17

SEQ ID NO: 78 - DNA, 489 bp, Escherichia coli; identical to SEQ ID NO: 18
  source 1..489: mol_type = other DNA

SEQ ID NO: 79 - protein, 162 aa, Shigella sonnei; identical to SEQ ID NO: 19

SEQ ID NO: 80 - DNA, 486 bp, Shigella sonnei; identical to SEQ ID NO: 20
  source 1..486: mol_type = other DNA

SEQ ID NO: 81 - protein, 163 aa, Citrobacter pasteurii; identical to SEQ ID NO: 21

SEQ ID NO: 82 - DNA, 489 bp, Citrobacter pasteurii; identical to SEQ ID NO: 22
  source 1..489: mol_type = other DNA

SEQ ID NO: 83 - protein, 163 aa, Enterobacter timonensis; identical to SEQ ID NO: 23

SEQ ID NO: 84 - DNA, 489 bp, Enterobacter timonensis; identical to SEQ ID NO: 24
  source 1..489: mol_type = other DNA

SEQ ID NO: 85 - protein, 163 aa, Pluralibacter gergoviae; identical to SEQ ID NO: 25

SEQ ID NO: 86 - DNA, 489 bp, Pluralibacter gergoviae; identical to SEQ ID NO: 26
  source 1..489: mol_type = other DNA

SEQ ID NO: 87 - protein, 162 aa, Buttiauxella sp; identical to SEQ ID NO: 27

SEQ ID NO: 88 - DNA, 486 bp, Buttiauxella sp; identical to SEQ ID NO: 28
  source 1..486: mol_type = other DNA

SEQ ID NO: 89 - protein, 163 aa, Kosakonia sacchari; identical to SEQ ID NO: 29

SEQ ID NO: 90 - DNA, 489 bp, Kosakonia sacchari; identical to SEQ ID NO: 30
  source 1..489: mol_type = other DNA

SEQ ID NO: 91 - protein, 162 aa, Synthetic construct; identical to SEQ ID NO: 31

SEQ ID NO: 92 - DNA, 489 bp, Synthetic construct; identical to SEQ ID NO: 32
  source 1..489: mol_type = other DNA

SEQ ID NO: 93 - protein, 162 aa, Synthetic construct; identical to SEQ ID NO: 33

SEQ ID NO: 94 - DNA, 489 bp, Synthetic construct; identical to SEQ ID NO: 34
  source 1..489: mol_type = other DNA

SEQ ID NO: 95 - protein, 162 aa, Synthetic construct; identical to SEQ ID NO: 35

SEQ ID NO: 96 - DNA, 489 bp, Synthetic construct; identical to SEQ ID NO: 36
  source 1..489: mol_type = other DNA

SEQ ID NO: 97 - protein, 210 aa, Escherichia coli; identical to SEQ ID NO: 39

SEQ ID NO: 98 - DNA, 633 bp, Escherichia coli; identical to SEQ ID NO: 40
  source 1..633: mol_type = other DNA

SEQ ID NO: 99 - protein, 169 aa, Escherichia coli [sequence omitted]

SEQ ID NO: 100 - DNA, 510 bp, Escherichia coli; identical to SEQ ID NO: 42
  source 1..510: mol_type = other DNA

SEQ ID NO: 101 - protein, 848 aa, Escherichia coli; identical to SEQ ID NO: 43

SEQ ID NO: 102 - DNA, 2547 bp, Escherichia coli; identical to SEQ ID NO: 44
  source 1..2547: mol_type = other DNA

SEQ ID NO: 103 - protein, 275 aa, Escherichia coli; identical to SEQ ID NO: 45

SEQ ID NO: 104 - DNA, 828 bp, Escherichia coli; identical to SEQ ID NO: 46
  source 1..828: mol_type = other DNA

SEQ ID NO: 105 - protein, 442 aa, Leptospira interrogans; identical to SEQ ID NO: 47

SEQ ID NO: 106 - DNA, 1329 bp, Leptospira interrogans; identical to SEQ ID NO: 48
  source 1..1329: mol_type = other DNA

SEQ ID NO: 107 - DNA, 97 bp, Synthetic construct; identical to SEQ ID NO: 49
  source 1..97: mol_type = other DNA

SEQ ID NO: 108 - DNA, 36 bp, Synthetic construct; identical to SEQ ID NO: 50
  source 1..36: mol_type = other DNA

SEQ ID NO: 109 - protein, 369 aa, Bacillus subtilis
MKRHYEAVVIGGGIIGSAIAYYLAKENKNTALFESGTMGGRTTSAAAGMLGAHAECEERDAFFDFAMHSQRLYKGLGEELYALSGVDIRQHNGGMFKLAFSEEDVLQLRQMDDLDSVSWYSKEEVLEKEPYASGDIFGASFIQDDVHVEPYFVCKAYVKAAKMLGAEIFEHTPVLHVERDGEALFIKTPSGDVWANHVVVASGVWSGMFFKQLGLNNAFLPVKGECLSVWNDDIPLTKTLYHDHCYIVPRKSGRLVVGATMKPGDWSETPDLGGLESVMKKAKTMLPAIQNMKVDRFWAGLRPGTKDGKPYIGRHPEDSRILFAAGHFRNGILLAPATGALISDLIMNKEVNQDWLHAFRIDRKEAVQI

SEQ ID NO: 110 - DNA, 1110 bp, Bacillus subtilis
  source 1..1110: mol_type = other DNA
atgaaaaggcattatgaagcagtggtgattggaggcggaattatcggttccgcaattgcttattatttggcaaaggaaaacaaaaacaccgcattgtttgaaagcggaacaatgggcggcagaacgacaagtgccgctgccggaatgctgggcgcccatgccgaatgcgaggaacgtgacgcgttttttgatttcgctatgcacagtcagcgtctgtacaaaggtcttggagaagagctttatgcattatccggtgtggatatcaggcagcataacggcggtatgtttaagcttgcattttctgaagaagatgtgctgcagctgagacagatggacgatttggactctgtcagctggtattcaaaagaagaggtgttagaaaaagagccgtatgcgtctggtgacatctttggtgcatcttttattcaggatgatgtgcatgtggagccttattttgtttgcaaggcatatgtgaaagcagcaaaaatgcttggggcggagatttttgagcatacgcccgtcctgcatgtcgaacgtgacggtgaagccctgttcatcaagacccctagcggagacgtatgggctaatcatgttgtcgttgccagcggggtgtggagcggaatgttttttaaacagcttggactgaacaatgcttttctccctgtaaaaggggagtgcctgtccgtttggaatgatgatatcccgctgacaaaaacgctttaccatgatcactgctatatcgtaccgagaaaaagcggaagactggttgtcggcgcgacaatgaagccgggggactggagtgaaacaccggatcttggcggattggaatctgttatgaaaaaagcaaaaacgatgctgccggctatacagaatatgaaggtggatcgtttttgggcgggactccgtccgggaacaaaggatggaaaaccgtacatcggcagacatcctgaggacagccgtattttatttgcggctggccatttcagaaacgggatcctgcttgctcccgcaacgggcgctttgatcagtgatctcatcatgaataaagaggtcaaccaagactggctgcacgcattccgaattgatcgcaaggaggcggttcagatatga

SEQ ID NO: 111 - protein, 365 aa, Pseudomonas putida
MSKQVVVVGGGVIGLLTAFNLAAKVGQVVVCDQGEVGRESSWAGGGIVSPLYPWRYSPAVTALAHWSQDFYPQLGERLFASTGVDPEVHTTGLYWLDLDDEAEALAWAAREQRPLSAVDISAAYDAVPVLGPGFKHAIYMAGVANVRNPRLVKSLKAALLALPNVSLREHCQITGFVQEKGRVTGVQTADGVLAADEVVLSAGAWSGDLLRTLGLELPVEPVKGQMILFKCAEDFLPSMVLAKGRYAIPRRDGHILVGSTLEHAGYDKTPTADALESLKASAVELLPELEGATVVAHWAGLRPGSPEGIPYIGPVPGHEGLWLNCGHYRNGLVLAPASCQLFTDLLTGAEPIIDPAPYAPEGRLG

SEQ ID NO: 112 - DNA, 1098 bp, Pseudomonas putida
  source 1..1098: mol_type = other DNA
atgagcaagcaagtagtggtggttggcggcggggtgatcggcctgctgacggcattcaacctggcagccaaggtcggccaggtggtggtgtgcgatcagggcgaggttggccgagagtcgtcctgggctggcggaggcattgtctcgccgttgtacccgtggcgctacagccccgccgtgaccgctctggcgcactggtcgcaggacttttatccacagctgggcgagcgtctgttcgccagcaccggtgtcgacccggaagttcacaccaccgggctgtactggctcgacctggacgacgaggccgaagcgttggcctgggccgcgcgcgagcagcggccgctcagtgccgtggatatttcggcggcctacgatgcggtgccggtgctgggcccgggcttcaagcacgccatctacatggctggtgtggccaatgtgcgcaacccacgcttggtcaagtcgctgaaggcggcgttgctggcgctgccgaacgtgagcttgcgcgaacactgccagatcaccggcttcgtgcaggaaaaggggcgcgtgactggtgtacagacggctgatggcgtactggcggcggatgaggttgtgctcagcgcaggcgcctggagtggcgacctgttgcgtacccttggccttgaactgccggtggagcccgtgaagggccagatgattctgttcaagtgtgctgaagacttcttgccgagcatggtgctggccaaggggcgctatgcaatcccgcgacgtgacgggcacattctggtagggagcacgctggaacacgccgggtatgacaagaccccgaccgcggatgcgctggaaagcctgaaggcatcggcggtggagttgttgcctgagcttgaaggcgcgacagtggtggcgcactgggcgggactgcggccggggtcgccagaaggcatcccttacattgggcctgtgccagggcatgaagggttgtggctgaactgcgggcattatcgcaatgggctggtgctggcgccggcttcgtgccagctgtttaccgatttgctgacaggggccgagccgattatcgacccggcgccgtatgcgccagaagggcgtttgggctga

SEQ ID NO: 113 - protein, 371 aa, Magnetococcus marinus
MEHVVIVGAGVMGTACAFRLLEQGYRVTLLEKALPGAESSAAAAGILGAQSEVSGGGAFLDLCVASRGMFRDFVGRLQDVSGVHCGYEESGVLEVAMDPAEGRLAAGRAEWMLERDLRVEILDRDEARRLEPALSERIIGANFYPDDHQVEPVALSRALAAAVARLGGQFRSGVQVTGLHVQGERVVGVASRDEVIHGDHVIIAGGAWSSMIGGLPPLRTQVKPMSGQILQVEMRPPAFRHVVYGYKGYIVPRADGRVVMGSTLEDRGFDKAVTTEGLQRITQMALEMVPLLKQARMTDAWAGLRPATGDGHPLLGQGPWPGLYFATGHYRNGILMTPITAEVTTQFIMGQVPPWDLTPFTPIHLMKEGHE

SEQ ID NO: 114 - DNA, 1116 bp, Magnetococcus marinus
  source 1..1116: mol_type = other DNA
atggaacatgttgtgattgtcggggctggggtgatggggaccgcatgtgcctttcgtctgttggagcaggggtatcgcgtcacgctcttggaaaaagccctgcctggggccgaatcctcagccgctgccgcgggcatcttgggggctcaatccgaggtgtcagggggaggggcttttttagatctgtgtgtcgccagtcggggcatgtttcgggattttgtagggcgtttgcaggatgtaagcggcgtacattgtggttatgaagagagtggggtgctggaagttgccatggatcctgccgaggggcgtttggcggctggacgggcggagtggatgttagagcgcgatttacgggttgaaatattggaccgggacgaagcgcgtcggttggaacctgcactgagcgagcgcatcataggagcaaacttttatcccgatgatcaccaagtggagccggtggccttgtcgcgcgccttggccgccgccgtagcgcgcttaggggggcagtttcgcagcggggtacaggtgacagggctgcatgtacagggggagcgggtggtgggggtggccagccgggatgaggtgattcatggggatcatgtcatcattgccgggggggcctggagcagcatgattggggggttgcccccgctgcgcacccaggtgaagcctatgtcgggtcagattttgcaggttgagatgcgccccccggcctttcgacatgtggtgtatggctacaaaggttacattgtgccccgtgccgatgggcgggtggtcatgggttcaaccctggaagaccgaggctttgataaagccgtaaccaccgagggcttgcagcgcatcacacagatggcgctggagatggtgcctctgcttaagcaggcccgcatgaccgatgcttgggcagggctgcggcctgccacaggagatggccaccccttgctgggacaaggaccttggccggggctctattttgccacggggcactataggaatggcattttaatgacacccattactgctgaagtgaccactcagttcattatgggtcaggtacccccttgggatttgaccccctttacgcccatacatttgatgaaagagggtcatgaataa

SEQ ID NO: 115 - protein, 379 aa, Synechococcus elongatus
MAFEVAVFGGGVIGLAIALELRLRGAMVQVYSQNSQAAAGRVAAGMLAPQSEGIEVGPMLDLGLRSRSLYASWTQQIEQLSGQDSGYWPCGILVPLSDAANRDRYPHPAESPGQWLSAADLRDFQPALRSDLIGGWWFPQEGQVDSRRALYPALRAAAIASGVILHEGVALQELVVTGDRLQSATTEQGPVQADAYVLATGAWSGDWLQLPVYPVKGQMFSLQAHPDLLRHVLFGEQAYIVPRRDGLIVVGATVEATAGFSPGNTAGPLQSLMAEAIALVPALADCPLVETWWGYRPATPDEWPILGQGPAENLFLATGHYRNGMLLAPITAQLLADQILDHRTDPLLHAFRYDRFSIHDSSTHQPLPALAGLSASTSQ

SEQ ID NO: 116 - DNA, 1140 bp, Synechococcus elongatus
  source 1..1140: mol_type = other DNA
atggcgttcgaggtagccgtctttgggggcggcgtcattggcttggcgatcgcgctagaactgcgattgcggggcgcgatggttcaggtctacagccaaaacagtcaggcggcggcaggccgagtcgcggcggggatgttggcaccccagtcggaaggcatcgaagtcgggcccatgctggatctggggctgcgcagccgatcgctctacgccagctggacgcaacagattgaacagctcagcggtcaagacagcggctactggccctgtggcattttggtgccgctcagtgatgctgccaatcgcgatcgctatcctcacccggcagaatcgccgggacagtggctctcggcagcagacttacgagactttcagccagctctgcgctccgatttaattggtggctggtggtttccccaagaaggacaagtcgacagtcgccgcgcgctctatcccgctttacgagctgccgcgatcgccagtggcgtcatcctccacgaaggcgttgctcttcaagagttagtagtgacgggcgatcgcctgcaatcggcgacgacggagcaaggcccagttcaagctgatgcctacgttctggcaacaggtgcttggtccggtgactggctgcaactgccggtctatcccgttaagggtcaaatgttttcgctgcaggcgcatcccgatttgctgcgccacgtcttgtttggggagcaggcttacatcgtgccgcggcgcgatggcctgattgtcgtcggtgccaccgtagaagcaacagctggcttcagtcccggcaatactgctgggccgctacagagcttgatggctgaagcgatcgcgcttgttccggctctggcggactgtccgctggttgaaacgtggtggggctatcgtcccgcaactccggatgaatggccgattcttgggcaaggccccgctgaaaacctattcttggcgaccggccactaccgcaacggtatgctgctcgccccaattaccgctcagctgctcgctgaccaaattctcgaccaccgcacggatccactgcttcatgccttccgctacgaccgcttctccatccatgactccagcacccatcaacccttacccgctcttgcaggcttgtcagcgtcaacgagtcagtga

SEQ ID NO: 117 - DNA, 90 bp, Synthetic construct
  source 1..90: mol_type = other DNA
tcgcctaaagtaatgttgttgagaaaaccggtgagacggtcgctcaaatcacgccacagcgcgtaggtcaggcatttatcgccgccctgg

SEQ ID NO: 118 - DNA, 21 bp, Synthetic construct
  source 1..21: mol_type = other DNA
aatgcttgacgttgcgctcaa

SEQ ID NO: 119 - DNA, 22 bp, Synthetic construct
  source 1..22: mol_type = other DNA
gtggttgcggagtagtcgagat

SEQ ID NO: 120 - DNA, 90 bp, Synthetic construct
  source 1..90: mol_type = other DNA
cttggcaaaccgcaaacagacccgactctctaatagtgattgtctcattgccacattcataagtacccatccaagagcacgcttattcac

SEQ ID NO: 121 - DNA, 22 bp, Synthetic construct
  source 1..22: mol_type = other DNA
ggcgttatctggctctggagaa

SEQ ID NO: 122 - DNA, 22 bp, Synthetic construct
  source 1..22: mol_type = other DNA
acgagaacagccgacaatctga

SEQ ID NO: 123 - DNA, 75 bp, Synthetic construct
  source 1..75: mol_type = other DNA
gttggcggaatcacagtcatgacgggtagcaaatcaggcgatacgtcttggcattacacgtcttgagcgattgtg

SEQ ID NO: 124 - DNA, 77 bp, Synthetic construct
  source 1..77: mol_type = other DNA
cggataagcctcgctttccggcacgttcatcacgaaaaatattgctgtaatgggaattagccatggtccatatgaat

SEQ ID NO: 125 - DNA, 22 bp, Synthetic construct
  source 1..22: mol_type = other DNA
cgaaatatcccgaatgccgcgt

SEQ ID NO: 126 - DNA, 22 bp, Synthetic construct
  source 1..22: mol_type = other DNA
cgtcgcacggcaccatattcca

SEQ ID NO: 127 - DNA, 63 bp, Synthetic construct
  source 1..63: mol_type = other DNA
tggagccacaatcttcgcccccaggctgccgctttcattatgacggaagcggttttcatcaat

SEQ ID NO: 128 - DNA, 90 bp, Synthetic construct
  source 1..90: mol_type = other DNA
aagcgcgcgatccacacgcaattgatttatggcatccagtcactattacagagtctcaatatagaggtacaagacgtatcgcctgatttg

SEQ ID NO: 129 - DNA, 90 bp, Synthetic construct; identical to SEQ ID NO: 120
  source 1..90: mol_type = other DNA

SEQ ID NO: 130 - DNA, 90 bp, Synthetic construct
  source 1..90: mol_type = other DNA
acgagatgcggcccaatttactgcttaggagaagatcatgtgatagtaagataaactgaaatctctggtttccgacgacaagaaggatac

SEQ ID NO: 131 - skipped

SEQ ID NO: 132 - DNA, 91 bp, synthetic construct
  source 1..91: mol_type = other DNA
gagttggtagctcttgatccggcaaacaaaccaccgttggtagcggtggtttttttgtttgcaagcagcagattacgcgcagaaaaaaagg

SEQ ID NO: 133 - DNA, 45 bp, synthetic construct
  source 1..45: mol_type = other DNA
tcataaaaaatttatttgctttgtgagcggataacaattataata

SEQ ID NO: 134 - DNA, 29 bp, synthetic construct
  source 1..29: mol_type = other DNA
ctgacgctttttatcgcaactctctactg

SEQ ID NO: 135 - DNA, 1083 bp, synthetic construct
  source 1..1083: mol_type = other DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 136 - DNA, 36 bp, synthetic construct
  source 1..36: mol_type = other DNA
ttgacaattaatcatccggctcgtagtgtctgtgga

SEQ ID NO: 137 - DNA, 80 bp, synthetic construct
  source 1..80: mol_type = other DNA
ccaggcatcaaataaaacgaaaggctcagtcgaaagactgggcctttcgttttatctgttgtttgtcggtgaacgctctc

SEQ ID NO: 138 - DNA, 36 bp, synthetic construct; identical to SEQ ID NO: 136
  source 1..36: mol_type = other DNA

SEQ ID NO: 139 - DNA, 72 bp, synthetic construct
  source 1..72: mol_type = other DNA
ttgacaattaatcatccggctcgtagtgtttgtggatggcagtggctagcggccgcagaggttatttcactc

SEQ ID NO: 140 - protein, 347 aa, Escherichia coli
MSVMFDPDTAIYPFPPKPTPLSIDEKAYYREKIKRLLKERNAVMVAHYYTDPEIQQLAEETGGCISDSLEMARFGAKHPASTLLVAGVRFMGETAKILSPEKTILMPTLQAECSLDLGCPVEEFNAFCDAHPDRTVVVYANTSAAVKARADWVVTSSIAVELIDHLDSLGEKIIWAPDKHLGRYVQKQTGGDILCWQGACIVHDEFKTQALTRLQEEYPDAAILVHPESPQAIVDMADAVGSTSQLIAAAKTLPHQRLIVATDRGIFYKMQQAVPDKELLEAPTAGEGATCRSCAHCPWMAMNGLQAIAEALEQEGSNHEVHVDERLRERALVPLNRMLDFAATLRG

SEQ ID NO: 141 - protein, 554 aa, Thermotoga maritima
MRSDVIKKGLERVPHRSLLKALGITDDEMRRPFIGIVSSWNEIIPGHVHLDKVVEAVKAGVRMAGGVPFVFPTIGICDGIAMDHRGMKFSLPSRELIADSIEIVASGFPFDGLVFVPNCDKITPGMMMAMGRLNIPSVLISGGPMLAGRYNGRDIDLITVFEAVGGYKVGKVDEETLKAIEDLACPGAGSCAGLFTANTMNSLAEALGIAPRGNGTVPAVHAKRLRMAKEAGMLVVELVKRDVKPRDIVTLDSFMNAVMVDLATGGSTNTVLHLKAIAESFGIDFDIKLFDELSRKIPHICNISPVGPYHIQDLDDAGGIYAVMKRLQENGLLKEDVMTIYLRKIGDLVREAKILNEDVIRPFDNPYHKEGGLGILFGNLAPEGAVAKLSGVPEKMMHHVGPAVVFEDGEEATKAILSGKIKKGDVVVIRYEGPKGGPGMREMLSPTSAIVGMGLAEDVALITDGRFSGGSHGAVIGHVSPEAAEGGPIGIVKDGDLIEIDFEKRTLNLLISDEEFERRMKEFTPLVKEVDSDYLRRYAFFVQSASKGAIFRKP

SEQ ID NO: 142 - protein, 373 aa, Acidobacterium capsulatum
MEVASAVAEPDACSLDHYLSLPDHSMDERIAAARARLGASTVLLGHHYQRDEVVRFADFTGDSYKLSKIAAETEAKYVVFCGVHFMAESADVLGRSEQHVILPDLNAGCSMADMAEISQVEACWETLLGAGLGAEQVMPLTYMNSTAAIKAFCGERGGLVCTSSNARKAFEWAFARAEKILFLPDQHLGRNTAFAMGIGLDEMVVFDPWQIGGGVPAEKLRAAKVILWKGHCSVHQRFLPEHVDRVRAKYEGIRVVVHPECRWEVCQKADALGSTERIIAEVEEAPEGTMFAIGTEIHLVNRLAKRFAPLGKKIITLDDTGCLCTTMYRISPQHLAWALENLLEGRVVNQIKVTDEVKHWARVALDRMLEIRG

SEQ ID NO: 143 - protein, 322 aa, Aquifex aeolicus
MVQLALKEEKELTKEEIKELQKEVRRLAKEKNAVILAHYYQRPEVQDIADFVGDSLELARKASQTDADIIVFCGVRFMCETAKIVNPEKKVLHPNPESGCPMADMITAKQVRELREKHPDAEFVAYINTTADVKAEVDICVTSANAPKIIKKLEAKKIVFLPDQALGNWVAKQVPEKEFIIWKGFCPPHFEFTYKELEKLKEMYPDAKVAVHPECHPRVIELADFVGSTSQILKYATSVDAKRVIVVTEVGLKYTLEKINPNKEYIFPQSMNYCGTVYCCTMKGITLPKVYETLKNEINEVTLPKDIIERARRPIERMLELS

SEQ ID NO: 144 - protein, 368 aa, Bacillus subtilis
MSILDVIKQSNDMMPESYKELSRKDMETRVAAIKKKFGSRLFIPGHHYQKDEVIQFADQTGDSLQLAQVAEKNKEADYIVFCGVHFMAETADMLTSEQQTVVLPDMRAGCSMADMADMQQTNRAWKKLQHIFGDTIIPLTYVNSTAEIKAFVGKHGGATVTSSNAKKVLEWAFTQKKRILFLPDQHLGRNTAYDLGIALEDMAVWDPMKDELVAESGHTNVKVILWKGHCSVHEKFTTKNIHDMRERDPDIQIIVHPECSHEVVTLSDDNGSTKYIIDTINQAPAGSKWAIGTEMNLVQRIIHEHPDKQIESLNPDMCPCLTMNRIDLPHLLWSLEQIEKGEPSGVIKVPKAIQEDALLALNRMLSIT

SEQ ID NO: 145 - protein, 394 aa, Corynebacterium glutamicum
MPDVYGPGASQNDPIPAHAPRQQVLPEEYQRASDDELHRRIREAKDTLGDKVVILGHFYQRDEVIQHADFVGDSFQLARAAKTRPEAEAIVFCGVHFMAETADLLSTDEQSVILPNLAAGCSMADMADLDSVEDCWEQLTSIYGDDTLIPVTYMNSSAALKGFVGEHGGIVCTSSNARSVLEWAFERGQRVLFFPDQHLGRNTAKAMGIGIDQMPLWNPNKPLGGNTVSELENAKVLLWHGFCSVHKRFTVEQINKARAEYPDVHVIVHPESPMPVVDAADSSGSTDFIVKAIQAAPAGSTFAIGTEINLVQRLAAQYPQHTIFCLDPVICPCSTMYRIHPGYLAWALEELVAGNVINQISVSESVAAPARVALERMLSVVPAAPVTPSSSKDA

SEQ ID NO: 146 - protein, 352 aa, Pseudomonas putida
MTQISERLLVQAHLDAKQPNPLTAEQEAEYRAAIAAELKAQNAVLVAHYYCDPVIQALAEETGGCVSDSLEMARFGKNHPAETVIVAGVRFMGETAKILTPEKRVLMPTLEATCSLDLGCPVEEFSAFCDQHPERTVVVYANTSAAVKARADWVVTSSCALEIVESLMDNGETIIWGPDQHLGRYIQKQTGADMLLWDGACIVHEEFKSRQLADMKALYPDAAILVHPESPEAVIELADAVGSTSQLIKAAQTLPNKTFIVATDRGIFYKMQQLCPDKEFVEAPTAGNGAACRSCAHCPWMAMNTLERVLDCLRKGGDEIFVDPALVPKAIKPLNRMLDFTQAARLKLSGNA

SEQ ID NO: 147 - protein, 311 aa, Sulfolobus solfataricus
MTRVEELLSQIKNLKTEKNAIILGHNYMEYSVQLVSDFTGDSYDLAVKAMKTNAKIILFAGVYFMAEQASALNPNKKVLSPDPNAGCTLSDSLDVKTLQEYKEKYPNAPVVLYINTSIYAKALADYIVTSSTAVKVVQKLNADTILFGPDANLANYVQQKVPNKKIIKVPPNGRCIVHANYTKQLVELARKKYPNALLMAHPEAPLEILESADFVGSTNQMIQFSKENKNEEFIVATEIGMINALKIKNPSKKFYPLVTTEACACARCPYMNMINLEKVKRSLEEEVYEVKVPEDIAERVKRAFENTMKLL

SEQ ID NO: 148 - protein, 320 aa, Synechococcus elongatus
MFLAADRPTTDLPADLPAAIAALKQELNAVILAHYYQEAAIQDVADYIGDSLGLSRQAAAADADVIVFAGVHFMAETAKILNPQRQVLLPDLAAGCSLADSCPPEAFAAFKAAHPNHIVISYINCTAEIKALSDIICTSSNAVKIVQQIPVDQPIIFAPDRNLGRYVMQQTGRDLVLWDGSCIVHETFSEQRLLELQARHPDAEIIAHPECETPVLDQARFIGSTTALLNYSLNSPSREFIVVTEPGIIHQMQQAAPEKTFIPAPPQDTTCACNECPFMRLNTLEKLYLCMRDRRPEIQIPEETRLAALRPIERMLAMSA

SEQ ID NO: 149 - protein, 310 aa, Thermus thermophilus
MGRMRGEALAQEVLRLKRERNAVILAHSYQLPEVQEVADFVGDSLGLAREAQRTRAEVIVFCGVHFMAETAAILNPEKTVLLPDLEAGCSLADSIRPEDVLAWKAKHPDGIVVAYVNTKAEVKALADVCVTSANAVEVVSRLPQDRPIYFVPDMFLGAHVARVTGRRLDLFPGECHVHAGIREEHLKALLEAHPGAEFLIHPECGCGSGCLYLKPDAKMLSTEGMVRYAKGAEAREFVVATEVGILHRLKKEAPEKAFFPVKPDAVCEYMKRITLEKVYLSLKEMRHVVRVPEEVAGRARRALEAMVAVG

SEQ ID NO: 150 - protein, 718 aa, Arabidopsis thaliana
MALALSVAPTSSSLSSLLSRTPNPSPNFRTTHLNFGSQRRIYTINPLLRSFKCLQSSSRDVNASPFSISAIASSSSSSQTTELVPYKLQRLVKEFKSLTEPIDRLKWVLHYASLLPQMPESSKTESNRVMGCTARVWLDAELGQDGKMRFCADSDSDVSKGMCSCLIQVLDEASPVEVMELKTEDLAELNVGLLGGERSRVNTWYNVLVSMQKKTRRLVAEREGKVPSFEPFPSLVLTAHGIEAKGSFAQAQAKYLFPEESRVEELVNVLKEKKIGVVAHFYMDPEVQGVLTAAQKHWPHISISDSLVMADSAVTMAKAGCQFITVLGVDFMSENVRAILDQAGFEKVGVYRMSDETIGCSLADAASAPAYLNYLEAASRSPPSLHVVYINTSLETKAFAHELVPTITCTSSNVVQTILQAFAQMPELTVWYGPDSYMGANIVKLFQQMTLMTNEEIANIHPKHSLDSIKSLLPRLHYFQEGTCIVHHLFGHEVVERIKYMYCDAFLTAHLEVPGEMFSLAMEAKKREMGVVGSTQNILDFIKQKVQEAVDRNVDDHLQFVLGTESGMVTSIVAVIRSLLGSSANSKLKVEVVFPVSSDSMTKTSSDSSNSIKVGDVALPVVPGVAGGEGCSIHGGCASCPYMKMNSLSSLLKVCHKLPDLENVYGGFIAERFKRQTPQGKLIADVGCEPILHMRHFQANKELPDKLVHQVLSCESKR

SEQ ID NO: 151 - protein, 540 aa, Escherichia coli
MNTLPEHSCDVLIIGSGAAGLSLALRLADQHQVIVLSKGPVTEGSTFYAQGGIAAVFDETDSIDSHVEDTLIAGAGICDRHAVEFVASNARSCVQWLIDQGVLFDTHIQPNGEESYHLTREGGHSHRRILHAADATGREVETTLVSKALNHPNIRVLERSNAVDLIVSDKIGLPGTRRVVGAWVWNRNKETVETCHAKAVVLATGGASKVYQYTTNPDISSGDGIAMAWRAGCRVANLEFNQFHPTALYHPQARNFLLTEALRGEGAYLKRPDGTRFMPDFDERGELAPRDIVARAIDHEMKRLGADCMFLDISHKPADFIRQHFPMIYEKLLGLGIDLTQEPVPIVPAAHYTCGGVMVDDHGRTDVEGLYAIGEVSYTGLHGANRMASNSLLECLVYGWSAAEDITRRMPYAHDISTLPPWDESRVENPDERVVIQHNWHELRLFMWDYVGIVRTTKRLERALRRITMLQQEIDEYYAHFRVSNNLLELRNLVQVAELIVRCAMMRKESRGLHFTLDYPELLTHSGPSILSPGNHYINR

SEQ ID NO: 152 - protein, 275 aa, Mannheimia succiniciproducens
MTLQQQIIKALGAKPQINAEEEIRRSVDFLKSYLQTYPFIKSLVLGISGGQDSTLAGKLCQMAINELRLETGNESLQFIAVRLPYGVQADEQDCQDAIAFIQPDRVLTVNIKGAVLASEQALREAGIELSDFVRGNEKARERMKAQYSIAGMTSGVVVGTDHAAEAITGFFTKYGDGGTDINPLYRLNKRQGKQLLAALACPEHLYKKAPTADLEDDRPSLPDEVALGVTYDNIDDYLEGKNVPQQVARTIENWYLKTEHKRRPPITVFDDFWKK

SEQ ID NO: 153 - protein, 237 aa, Escherichia coli
MRKITQAISAVCLLFALNSSAVALASSPSPLNPGTNVARLAEQAPIHWVSVAQIENSLAGRPPMAVGFDIDDTVLFSSPGFWRGKKTFSPESEDYLKNPVFWEKMNNGWDEFSIPKEVARQLIDMHVRRGDAIFFVTGRSPTKTETVSKTLADNFHIPATNMNPVIFAGDKPGQNTKSQWLQDKNIRIFYGDSDNDITAARDVGARGIRILRASNSTYKPLPQAGAFGEEVIVNSEY

SEQ ID NO: 154 - protein, 213 aa, Escherichia coli
MPPRALLLVDLQNDFCAGGALAVPEGDSTVDVANRLIDWCQSRGEAVIASQDWHPANHGSFASQHGVEPYTPGQLDGLPQTFWPDHCVQNSEGAQLHPLLHQKAIAAVFHKGENPLVDSYSAFFDNGRRQKTSLDDWLRDHEIDELIVMGLATDYCVKFTVLDALQLGYKVNVITDGCRGVNIQPQDSAHAFMEMSAAGATLYTLADWEETQG

SEQ ID NO: 155 - protein, 241 aa, Streptomyces sampsonii
MRTRLIGLLTAAASALTLSVLALGPTAQADTRAAADDFIVSEGQFESMFPNRNSFYSYSGLTDALSAYPGFTGTGSDTTRKQEAAAFLANVSHETGGLVHIVEQNEANYPHYCDTTQPFGCPAGNDKYYGRGPVQLSWNYNYKAAGDALGIDLLNNPDLVQNDASVAWKTGLWFWNTQSGAGSMTPHDAIVNGAGFGETIRAINGSIECNGGNPGQVQSRIDLYERFTDVLGVEPGDNLSC

SEQ ID NO: 156 - protein, 297 aa, Escherichia coli
MPPRRYNPDTRRDELLERINLDIPGAVAQALREDLGGTVDANNDITAKLLPENSRSHATVITRENGVFCGKRWVEEVFIQLAGDDVTIIWHVDDGDVINANQSLFELEGPSRVLLTGERTALNFVQTLSGVASKVRHYVELLEGTNTQLLDTRKTLPGLRSALKYAVLCGGGANHRLGLSDAFLIKENHIIASGSVRQAVEKASWLHPDAPVEVEVENLEELDEALKAGADIIMLDNFETEQMREAVKRTNGKALLEVSGNVTDKTLREFAETGVDFISVGALTKHVQALDLSMRFR

SEQ ID NO: 157 - protein, 448 aa, Pseudomonas denitrificans
MQDSSALPVRPHACPGLLRIVASRDGGICRIKLPCGQLDAQAARAIALAAERHADGVLEATNRANLQIRGVHAGAENALSRDLLAAGLGPRELAADDVRNLLVSPALGLDAGTAFDAQPLARQLLDLLERTPRFHGLSPKFGVQLDAGESLAMLEHPNDIWLSAMSEDAAPLFAFGLAGCPPQRKTDRPALAAVSAEQLPLLVETLLHLFLDLAGDGQTRMRHLREKLRDEALLERLQQRLPFLLRLGPLVDNWRRPAPQAFGYLGIRPQRQPERTLVGAGFVLGRLDSSILFGLADLTERFSGGQLRLTPWQSLILPDVAPADAPVALGALANLGLLIDAAQPLSRLIACSGASGCARGLADTKADALRLAERLRHAPPAGVHLCGCPRSCAAAHVASYTLLAVADDRYDLFRRADGVAGFGQPLARNLSLKAAGDLLAAPGAPTDA

SEQ ID NO: 158 - protein, 258 aa, Cornybacterium glutamicum
MSSDVVFQSVSVDSGVSVGTVSFPAGRVGASEWEMLADVAESQGDGVIYVTSLAQCQVRGGSYSHAEPCATVVATPGHFVALAREIAGAVRRELTVGLDAGDGAILRQSVDVGFLLVDASFHIHINGVSTGQSVAPDDVVEVVRGLADASELSVESVAELCTPVAPVSLPEAQGNPAPIGWLEHDGVVSLGAGIPGGRVEARLARFIAVIEAETTITPWNSLIIHDLHEGVAEQVVKVLAPMGLVFDANSPLLESPAL

SEQ ID NO: 159 - protein, 468 aa, Frankia canadensis
MPGWRTYAPPVPTSPRERVADACPGVLRTHAAADGALARLRLPGGLLDATAARALAACAREFGDGYLELTSRGNVQLRGLADGAATILAARARAAGLLPSDTHERVRNIVASPLSGRAVASWRDVTPLVEALDRALCADPALAALPGRILFALDDGSGDVAALPADLTLIALPAGRMLLRVAGEPRAVLLPERCAIPAILAAAHAFLDLLAATAPGDTPAWRIAELPTGHDRLLTAAQLAARRARTDDREPPAATIEIIQAGTAARSRGDASAVTPGPHRHSDGRYALTVLVPLGRLRADQLDLLAEIADEAAGDSRMVVTPWRTVVLRDLDPSASQGFGARLAAVGLVVDPASGWVGVTSCAGRPGCARALADVRRDAARYAAHVQGSTTSVHWSACARRCGRPAGDVVDVIADESGYRVHGGEELPADRWAALSRELASRHESASLQELMSTRAGTSGASTGREGD

SEQ ID NO: 160 - protein, 725 aa, Nostoc sp. CENA543
MLAGSTPCPGLFYATPAQDGILSRIRIPGGIINSKQCQAIADIADKYGGGYVDVTNRANLQIREIHQGIDAQTLQQLQQLGIGSHNASVDQIRNIMTSPTAGIDPQELIDTRPYVESWDKYITAHPTLSELSPKFSVCFDGGGLVSVCDRPNDITLAAVTIDNKIYFRLYITAGAKGQPPTATDIILHPEQTLKVLATFTDIYLSLCVYPTGTSPANGPLCEKNHPHTQRKPRLRQIINHIGLDNYLREVEQKLPFPLQRIDLTPLPPSLRGKGELETIFSPLVVKEGLGERSKTQYHHIGIHPQRQPGLYYIGLVLPLGRLETKQLRGLAKLSAKYGDGTLRLTPWQNVLLTDIPQHQLTDVQTEITDLKLDASPTNIKSALVACSGNQGCASAATDTKTHALAVAEYLQSRITLENPVNIHFSGCDKSCAQHTQSDIALVGVSEERYQLYVGTGENNQKFGRELYQDVSFTDLPALMEKILNVYKNKRLNSAESFGEFANRHTPNQLKQLLSRPSMPDYIRNANEIYRNSFAIIRAEANLDILPSDVAKVAVRLIHACGMTDIVTDIGYSTTAVNSARKAIAAGAPILCDCRMVADGVTRKRLPANNAVICTLNEPEVPELAKKLGNTRSAAALELWKPYLEGAIVAIGNAPTALFRLLEMLDAGCPKPAVILGFPVGFVGAAESKAALAADSRNVPFLTLHGRRGGSAIAAAAVNALATEEE

SEQ ID NO: 161 - protein, 460 aa, Rhizobium leguminosarum
MSIKAAKASDRTGEADLHGRPAATMRRRGACPALAAPMATGDGLLVRLRPVGGALTLSQFASLARSAAAHGNGILEITARGNLQIRGLRAETVGQLAADIDAAGITVPEGPAIETSPLHGIDPEEISDPAVMEMALRATLHDLFASPRLAPKLSLIVDGGGSFGLSALSADIRVVAQFGADWLVAINGDGETAMPVAIGPAEAAISAVGEILSLLATLGQGRRARDIDATLLRARFPAMDGIQFISSRAAVMPLAGSHRLDDGKIILGVRPEFGQMRASDLVALLDLAKARGATAIRLAPGRGFFLIGLSDDAVLAMQIAAARHGFSSQPGENTEHIAACAGAGACGSAFYETRTLARRLIAAAPALFDGSLTLHLSGCAKGCAHARPALTLTGSAEGYGLILNGLAADQPDERIAGGRIDFAIERLARSIEDNKDAGESTAACLTRLGAIGVSKALRQE

SEQ ID NO: 162 - protein, 464 aa, Mycoplana dimorpha
MIAAVDAGCDTDRSTAASGSISALRRGACPSLTAPMRTGDGLLVRLRPARPGIAPSDWIEIARLAAAHGNGLLEVTARGNLQVRGLTEQSVPKLAAGLAEAGIDLHSGVAVETPALSGIDPGEIADANPPAAAIRAAITACRPPLLLAPKLSIVVNGGGRLDLSAMVADVRLDAVRRDGALFWRLAAGGTLRSARPIAVVADRDAVAAVLAVLEALHGLGPAVRGRDVDVDAVRRQLGSMALPAEAAGATAPPASPIGRRGFGDTEILGLGLAYGQTSAARLEALMRGLQALGATEVRLAPQRALLVLGLAAERIEPAGALAAAEGFWTHADEPGNAIAVCAGAAGCASGHFDTKAAADLLVARGADLLDGSAAVHISGCGKGCAHPSAAALTLVGRADGVALVIGGRAGDATGGVVPADALGTAIDRLTALYRQEKRQGDPARDVLDRIGAAALAKAFQQDLQ

SEQ ID NO: 163 - protein, 374 aa, Rhodobacter sphaeroides
MNARVAAKGWCPGAWRPMASGDGLIVRVRPVLGRLTAAQVATLCDAAMECGSGMLELTSRANLQLRGVRGEAHEDLLARLWELGLIDADQEAETRRNLLVAPLWQAGDDTESTATALLERLADLPVLPAKFGFAIDAGEAPVLGAASADIRLERGVTGGMILRADGCAKGRPVTAGDAVDEIIRLARWFAETSGTAGRMARHPALREVEGSEPPAPSASLPVPGDTLLGSAWGVAFGQIDADRLARLVTDSGAEALRITPSRVVILEGGKAVGTEGFIGDRSNPLLTTDACPGAPLCGSATVETRKLARALAGHVAGLHVSGCAKGCARARAAPVTLVGRDGRFDLVSDGAAWDAPQRRGMTPAQVLELFGVAR

SEQ ID NO: 164 - protein, 421 aa, Granulicella tundricola
MRLGENFCPGILHAVPAKDGQLIRIRVPGGLISAAQLAAVASLAASHANGEIELTSRSNLQLRAIQPSSLPYIVTTLTEAGLLPSSAHDRVRNLVTSPFAGIDPAASLDTRPLIRALDQRLIANDLFLQLHPKFTFGIEANPTRFTYEVEDLGLQAFDSTHRLSLSLAGKDTGCSVAPEDAVVCLLTAAEACIDLAKQHNVPVRTKRLTAIPGVLETLLKTLSPLLEPTTNPARGPDVRRDVIGPHPALTPGHLNLVPSIPLGHLTASQATNIATLATQHNADLHLAPWRGIVLGNLTPKAAALIAPQLAALGLPLDATNGFEGIAACAGISRCDASLADVRADAARLATYLATRPHIPGWTIHLSGCEKQCALRHSASAALIATPAGYTLRLPNHPEAHSLPSAAAIASILQSHTEALAS

SEQ ID NO: 165 - protein, 455 aa, Sinorhizobium meliloti
MTSCAAPRADRSATGTSMRRGSCPSLAAPMQTGDGLLVRLRPAGDGLTPAEMRAIAGAAARCGSGVIEVTARGNLQIRGLTPESVPALAEAIGAAEIAIAEGVAIETPPLAGFDPDEIADPRPLARALRVAISGAGEALRLAPKLSIVVDGGGRFHLGSLTADLRVSALAHEGGVRWLFSLGGTARSAKPVALLESKDIVPMVLEILGDLTARGPASRARELDADLLRQRAAAGDALPRVDGPVFSPPPAGIHHFGGAGTVLGIGLAFAQTDAGSLVAFLRQAEELGANEIRLAPPHGLFVLGLSGETAAVAQRLASAHGFLVASGDPRNHIATCAGLACASARMDTKAMARLLLEAAPDLLDGSATVHLSGCPKGCASPTASPLTLVGAPSGYALVVNGTASVAPSAYRDENGIGSAFGALNALVRENKDAGESALSCLTRLGTARIAAAFEQG

SEQ ID NO: 166 - protein, 442 aa, Streptomyces cattleya
MAGSTPGAIIGMLAAMSTASTPPSSQAAAVSRDRGDACPGALRLHAADDGALARVRLPGGVLTTRQAEALGSAALRLGDGDLHLTSRGNVQLRGLPAGCGAELAGLLEAAGLLPSPGHERVRNVVASPLSGLDGHGLRDVRPWLTGLDALLCASEAARELSGRFLFALDDGRGDVAGLGADVTVRAVPDGDALVRLGASGTALRVPGGQAARATLTAAEVFLDAARASGTRAWRVAELALPAEELTAALGRRLTADGINHATGVPEATGTGGPLPGVTGDALSVHVPLGRLSAAQWRELTRAARGELRLTPWRGVVLPAPGLTPERAADALARLRAVGLVTDPDSPWPRVGACVGRPGCARSRADVRTDAAAALDAAGLPGLPLYWSGCERRCGHPVGDRIDVVAAEDGGYRVSVTASGRAPRTATVDHPSRLATVLAAITS

SEQ ID NO: 167 - protein, 457 aa, Pannonibacter phragmitetus
MSALKTPALAVGEAPRVPVRRGACPSLAAPMATGDGLLVRLRPQAPGLSPAEWMTMAGLARRTGNGLLEVTARGNLQIRGLTPESAAPLAEGLQAAGIVTRTGTAIEVPPLSGLDPSETADATELAEAIRQGIASHVPPLALAPKLAITVNGGGCFNLSTASADIRLDAVKAEGGVLWRLALAGDAGSAKPVALLPEEAAVARVMELLRALSQRGGHARGRDIAPDIEGSEDGAGPEAGFPAPVRPAGVIRSGALLVLALRLAYGRIHADELEALMAGLQHLGATEIRLAPDHALLVLGLTDETVEAAAALAGAQGLITSPSDPAHFISACPGTGFCAAARINTRQLAECLISAASAAEPGVLDGSCRVHVSGCSKGCAHPQAAALTVTGTEQGAAIVLGGRADSRPDLQLSPQRLETVLPRLQRGIELNRQPGQTAQHVLGASGAAILAMAGAERA

SEQ ID NO: 168 - protein, 246 aa, Pseudomonas denitrificans
MTGRLIGLGVGPGDPELLTLKALRLLRASPVIGYFVAKAKHHAGQGGNAFAIIEQHLLDEQQRLPLVYPVTTEKLEPPLTYEGVISDFYDTAAAQVAAHLDANRDVAVICEGDPFFYGSYMYLHDRLSQRYATEVVPGVCSMLGSAAVLGVPLVYRNQSLAVLSGVLPEDELKRRLADADAAVVMKLGRNFDKVRRVLRELGRDGGAHYVERATMREQRIVALDEVDPMASPYFSMIVIPGQRWNG

SEQ ID NO: 169 - protein, 251 aa, Pseudomonas denitrificans
MTVYFIGAGPGDPELITVKGQRLIRTCPVILYAGSLVPPAVLEGHSAERVVNTAELNLEEIVELLTQAHAEGKDVARVHSGDPSLYGAIGEQIRHLRERGIPYEIIPGVTATSACAAILGCELTLPEISQTLILTRYASRTRMPEGESLAELAQHQATLAIHLGIGQLANIVAELLPHYGADCPVAVIHRASWPDQDQVTGTLGDILPKAQAKGFRRTALILVGKVLGAEGFADSSLYSTGHTHLYRGGTL

SEQ ID NO: 170 - protein, 365 aa, Pseudomonas denitrificans
MREETPEEPRPLRSGYTTGSCATATSLAAARLLLTGVASDAAQIVLPKERSATLRLEFCRLTVDGAEAGTLKDAGDDPDVTHGALVFVRVALSAEPGVRFHAGEGVGTVTKPGLVLAVGEPAINPVPRQMMRDNLAVLAAECGYAGGFDVTINIEGGAELALKTMNPRLGILGGLSILGTTGIVRPFSCSAYIASIQQGIDVARANGFRHLAACTGNASEDAMRRRYGFDDTALIEMGDFAGAALKHLRKVPVERFSVCGGFGKISKLAAGHMDLHSRHSSIDLPQLAEWAAEIGASAELQQRMRAANTSQQALALCRAEGIALGDAVCARALAFARRIVPVEVVLEVFAIDRQGNLVGEALEVR

SEQ ID NO: 171 - protein, 242 aa, Pseudomonas denitrificans
MKRVLLLGGTGEALAIARRLGPRHLYSLAGLGKVPDDLACEVRVGGYGGAEGLAAFIREQGFELLLDATHPYAAQISANAARAAQLAGVPCWALRRPGWQAGAEDDWREVADWPALIAALEPFHRPLFTMGREPLEHLDETPAHQYWTVRCLQSLSGNERAEVLGARGPFSLDGERELFTRLGTDVLVSKNSGSQATEPKLQVARERGVPVLILARPTLPAVEREFDSLDSLWQALELLAAD

SEQ ID NO: 172 - protein, 208 aa, Pseudomonas denitrificans
MLDYIRDGQAIYRQSFATIRAEAELSAIPADLEKLAVRVIHACGMVDVVQDLRFSAGAGAIGRAALAAGAPILCDARMVAEGITRARLPAHNEVICTLNDPDVPALARELGNTRSAAALEHWREHLEGSVVVIGNAPTALFYLLEMLDAGAPKPALILGFPVGFVGAMESKDALAADSRGVPYVIVRGRRGGSAMAAAAVNALATEVE

SEQ ID NO: 173 - protein, 403 aa, Pseudomonas denitrificans
MTPWLTIVGIGEDGYAGLGKAARRALLAASEVIGAPRQLALLPHCLRASRQAWPSPFSLEPVLRRRGEPTCVLASGDPMLFGVGASLSRQIPPEEMQVISAPSSAALAAARLGWALQDCTLLSLLARPLAALNAQLHHGAKLLVLSNDGESPAAIAALLRERGFGPSRICVLEHLGGESERRMDGLASDWSPSETAALNIVAVQCVAGSDAVRLPLTIGLPDEAYRHDGQLTKRDVRAVTLARLAPTPGELLWDVGAGCGSIGIEWMRAHPSCRTLAIEANDGRQEHIRFNRDALGVPALQLVCGAAPDALQGLERPEAIFIGGGVTVPGVFERCWDQLKPGGRLVANAVTLQSEALLVSWRERIGGELTRLSVAQAQPLGGFDTWRAALPITLLEVHKPLAD

SEQ ID NO: 174 - protein, 549 aa, Pseudomonas denitrificans
MRIAIVILGQGALATARRLQGIYPQARVLGLKDRVEGADESYANFGETLRQLYRDDTPIIALCAAGIVIRTLAPLLAEKGAEPPVLAVAEDGSAVVPLLGGLGGVNRMAREMAAALDIAPAITTSGELRFGACLLDPPPGYALADIEQGKRFVSDLLGGDTLRIEGDALWLDELDLPLADDAAHTLRIDSRAEGIAHNELRIHPRIVLAHVTLVDSQLPERIRQGLAQAGLAEVSLAALVAPRCRMADPALAEAARELGVPLRFIHSDDELPPAMLHGDSFHLHRTATPGEAEGIGQPRGRLSVIGLGPGASDFMIPAVRHALDEAQDLLGYETYVNMAEPLRDDQVRHCTDNREEMQRARHAFELAASGRRVVVISSGDPGVFAMGAAVMEALHGSDDPDWHGVELEVLPGVSAALAAAAKAGAPLGHDFCLISLSDNLKPWETIEKRLDHAGAADLVMAFYNPISRARPWQLGRALEIVRRHRTPETLVVLGRDIGRPAEALRVVPLGELVPEMVDMRTLVIVGSSLTRRFPRAGGGDWVYTPRWYR

SEQ ID NO: 175 - protein, 1263 aa, Brucella melitensis
MHLLLAQKGTISDGNEAVDLGQTPGEIVFLSAADTELASIAQAHRLAPDMPSLRLANFLGLTHPMSVDAYVERTARHAKLIVVRIIGGETYWPYGLEALHACAITHGAKMAVLPGDDKPDHGLDRFSTIPAGERDDLWHYLIESGAANTHGFLAYCKALISGCEKPEAAAPLLKAGLWWPGEAVSSLTSVQRHWADPAAPVAAIILYRALVQSGQTQPVDALIAALQARGLNPLPIFVSSLKDPLSAAVVDGLFEDCPPDIVLNATGFAISSPGAERKPTVLDKRGNMVLQVIFSGTPKTVWEASQQGLLARDLAMNVALPEVDGRVLSRAVSFKSAQQFDASVEANIVTHEPHPDRVAFVAELAANWVRLKRKPPAERRVALILANYPNRDGRLGNGVGLDTPAGTVEVLRAMAAEGYRVGEIPADSDALMRALMAGPTNAARDGREIRETISLNQYKALFGKLALTIQAEVEARWGAPENDPYFARELDAFALPLMRFGETFVGIQPARGYNIDPKETYHSLDLVPPHGYIAYYAYLRAVAGVDAVVHMGKHGNLEWLPGKALALSQNCYPEAVFGPMPHIYPFIVNDPGEGTQAKRRASAVIIDHLTPPLTRAESYGPLKDLEALVDEYYEASGVDPRRLLRLKAQILDLVRDIGLDRDAGIHDHDDEDMALQKLDAYLCDLKEMQIRDGLHVFGLAPQARLLTDLLVALARVPRGIPVSLGGAPGDQSLQRAIAADAGLGEGFDPLDCNMAEPWAGAKPDMLLAASPATWRIAGDTVERIEILAAQLVAGEVPCPEDWTQTRAVLQSIEEQLRPMVVSCGPSEIDGFLAALSGRFVPPGPSGAPTRGRPDVLPTGRNFFSTDSRAVPTPAAWELGKKSAELLITRYTQDHGEWPTSFGLTAWGTSNMRTGGDDIAQALALIGVQPVWDMASRRVTGYEIVPPAKLARPRVDVTLRISGFFRDAFPEQIALFDKAVRAVGALDEDVEDNPIAARMKAEQARLVAGGADPQTAERRAGYRVFGSKPGAYGAGLQALIDENGWAGRNDLAEAWLVWGGYAYGAGEEGQAERGLLEERLRSVQAVVQNQDNREHDLLDSDDYYQFEGGMAATVESLTGAMPSVYHNDHSRPEKPVIRALEEELSRVVRGRAANPKWIAGVMRHGYKGAAEIAATVDYLFAFAATTGKVGNHHFEAVYQAYIADRAVHDFMAEKNPAALAETAAKLNEAIERGFWTPRSNSARFELENLSVHLQKLNPERAING

SEQ ID NO: 176 - protein, 328 aa, Brucella melitensis
MNKVERDIANLPDTTVSVREVFGIDSDMMVPAYAAGDSYVPELDPDYLFDRQTTLAILAGFAYNRRVMVSGYHGTGKSTHIEQVAARLNWPCVRVNLDSHVSRIDLVGKDAIVVKEGVQVTEFKDGILPWAYQHNVALVFDEYDAGRPDVMFVIQRVLESSGRLTLLDQSRVIRPHPAFRLFATANTVGLGDTTGLYHGTQQINQAQMDRWSIVTTLNYLPHDNEVNIVLVKAKHYQNAEGREIVNKMVRVADMTRQAFINGDLSTVMSPRTVITWAENAAIFNDVGFAFRLTFLNKCDELERATVAEFYQRAFGVELPESAANIVLA

SEQ ID NO: 177 - protein, 632 aa, Brucella melitensis
MSGIGDNSRDRKTGPVDSEPFKRAITACVRAISGDHEMEVAFSHDRPALSANRARLPDLPKRPTAHDIAVTRGLGDSMALRQARHNPRIHAALAPEGKQARAIFDAVEQARVEAIGARAMAGVADNLSTMLADKYSRANFSAVTTKEDAPLEEAVSLLLREKLTGRPAPAEAGQVLELWRDWIEQKASADIARLGENLEDQQAFARTVRDMLASMDMAEELSQEEPSDDEEQNDEQTPDSDENEEGGDENQEGSDSAESEESDSSSDEGEQGEMDAADASADEMDDSEDIDAETPGDTRRPNQPFANFAEHVDYKVFTREFDEEVEATDLCDEAELDRLRGFLDKQLANLQGVVGRLANRLQRRLMAQQNRSWDFDLEEGYLDSARLVRIVIDPTQPLSYKQERDTDFRDTVVTLVLDNSGSMGRPITVAATCADILARTLERCGVKVEILGFTTKAWKGGQSREAWLGRGKPANPGRLNDLRHIVYKSADAPWRRARRNLGLMMREGLLKENIDGEALIWAHQRLLGRPEQRKILMMISDGAPVDDSTLSVNPGNYLERHLRAVIEEIETRSPVELIAIGIGHDVTRYYQRAVTIVDAEELAGAMTEQLASLFEEQGAAASVRGRRRAGRR

SEQ ID NO: 178 - protein, 173 aa, Brucella melitensis
MQTVNNIISSVSTVESKAYRDAMSHYAGAVQIVTTAGAAGRRGLTLTAACSVSDNPPTILICLQKIHEENRIFIENGVFAINTLAGPHQQLADAFSGRIGLTQDERFELAAWEILATGAPVLKGALAAFDCRVVSVQDHSTHHVLFGEVVGLSSHAEEEALIYLNRRYHKLEL

SEQ ID NO: 179 - protein, 203 aa, Pseudomonas denitrificans
MSESPEKDERHRSRMQRKKTVVDEKIAQAQGERGVFLVHSGNGKGKSSSAFGMVARALGHGMKVGVVQFIKGAATTGEEAFFRRFPEEVSYHVMGEGFTWETQDRQRDIAKAREAWDVARQLLGDPQVGLVVLDEVNIALKHGYLALDSVLADIAARPPMQHVVATGRGVLPAMVEAADTVTEMGLVKHAFKAGIKAQKGVEF

SEQ ID NO: 180 - protein, 504 aa, Pseudomonas denitrificans
MTSPTGQAERLMDQGATLMVQGTTLMVQGTTSDAGKSTLVTALCRWLRRQGVAVAPFKPQNMALNSAVTADGGEIGRAQAVQAQACGLAPHTDMNPVLLKPNSDIGAQVVIHGRAVTSMNAAAYHDYKRVAMQAVLESHARLAAQYPVVMVEGAGSPAEINLRTNDIANMGFAEAVDCPVILVADIDRGGVFAHLVGTLELLSVSERARVKGFVINRFRGDIALLQNGLDWLEQRTGVPVLGVLPYLMDFHLEAEDAIDLRQTAKSGECLRVAVPVLPRISNHTDFDPLRLHPQVELSFVGPGQPRPPTDLIILPGSKSVRADLAFLREQGWDTAIQRHLRYGGKLLGICGGLQMLGQTLADPHGLEGAAGESDGLGLLDFRTVLEPEKQLRNVRGRLALEDAEVAGYEIHAGVSEGPALECPAVRLGDGRADGAVSADGQVLGTYLHGLFESPAACSALLRWAGLREVRSIDYHALRERDIERLADQVEAHLDTARLKALCGL

SEQ ID NO: 181 - protein, 196 aa, Escherichia coli
MSDERYQQRQQRVKEKVDARVAQAQDERGIIIVFTGNGKGKTTAAFGTATRAVGHGKKVGVVQFIKGTWPNGERNLLEPHGVEFQVMATGFTWDTQNRESDTAACREVWQHAKRMLADSSLDMVLLDELTYMVAYDYLPLEEVVQALNERPHQQTVIITGRGCHRDILELADTVSELRPVKHAFDAGVKAQIGIDY

SEQ ID NO: 182 - protein, 181 aa, Escherichia coli
MMILVTGGARSGKSRHAEALIGDSSQVLYIATSQILDDEMAARIEHHRQGRPEHWRTVERWQHLDELIHADINPNEVVLLECVTTMVTNLLFDYGGDKDPDEWDYQAMEQAINAEIQSLIAACQRCPAKVVLVTNEVGMGIVPESRLARHFRDIAGRVNQQLAAAANEVWLVVSGIGVKIK

SEQ ID NO: 183 - protein, 364 aa, Salmonella typhimurium
MALFNSAHGGNIREAATVLGISPDQLLDFSANINPLGMPVSVKRALIDNLDCIERYPDADYFHLHQALARHHQVPASWILAGNGETESIFTVASGLKPRRAMIVTPGFAEYGRALAQSGCEIRRWSLREADGWQLTDAILEALTPDLDCLFLCTPNNPTGLLPERPLLQAIADRCKSLNINLILDEAFIDFIPHETGFIPALKDNPHIWVLRSLTKFYAIPGLRLGYLVNSDDAAMARMRRQQMPWSVNALAALAGEVALQDSAWQQATWHWLREEGARFYQALCQLPLLTVYPGRANYLLLRCEREDIDLQRRLLTQRILIRSCANYPGLDSRYYRVAIRSAAQNERLLAALRNVLTGIAPAD

SEQ ID NO: 184 - protein, 203 aa, Escherichia coli
MRLWLIRHGETQANIDGLYSGHAPTPLTARGIEQAQNLHTLLHGVSFDLVLCSELERAQHTARLVLSDRQLPVQIIPELNEMFFGDWEMRHHRDLMQEDAENYSAWCNDWQHAIPTNGEGFQAFSQRVERFIARLSEFQHYQNILVVSHQGVLSLLIARLIGMPAEAMWHFRVDQGCWSAIDINQKFATLRVLNSRAIGVENA

SEQ ID NO: 185 - protein, 359 aa, Escherichia coli
MQILADLLNTIPAIDSTAMSRAQRHIDGLLKPVGSLGKLEVLAIQLAGMPGLNGIPHVGKKAVLVMCADHGVWEEGVAISPKEVTAIQAENMTRGTTGVCVLAEQAGANVHVIDVGIDTAEPIPGLINMRVARGSGNIASAPAMSRRQAEKLLLDVICYTQELAKNGVTLFGVGELGMANTTPAAAIVSTITGRDPEEVVGIGANLPTDKLANKIDVVRRAITLNQPNPQDGVDVLAKVGGFDLVGIAGVMLGAASCGLPVLLDGFLSYAAALAACQMSPAIKPYLIPSHLSAEKGARIALSHLGLEPYLNMEMRLGEGSGAALAMPIIEAACAIYNNMGELAASNIVLPGNTTSDLNS

SEQ ID NO: 186 - protein, 247 aa, Escherichia coli
MSKLFWAMLSFITRLPVPRRWSQGLDFEHYSRGIITFPLIGLLLGAISGLVFMVLQAWCGAPLAALFSVLVLVLMTGGFHLDGLADTCDGVFSARSRDRMLEIMRDSRLGTHGGLALIFVVLAKILVLSELALRGESILASLAAACAVSRGTAALLMYRHRYAREEGLGNVFIGKIDGRQTCVTLGLAAIFAAVLLPGMHGVAAMVVTMVAIFILGQLLKRTLGGQTGDTLGAAIELGELVFLLALL

SEQ ID NO: 187 - protein, 319 aa, Salmonella typhimurium
MTILAWCIAWVLDFIIGDPQHWPHPVRWIGRLITFVQRIVRRYCPGDKALRIGGGVMWVVVVGATWGVAWGVLALAQRIHPWFGWSVEVWMIFTTLAGRSLAHAAQEVERPLRKNDLAESRIKLSWIVGRDTSQLQPAQINRAVVETVAENTVDGIIAPLFFLFLGGAPLAMAYKAVNTLDSMVGYKHEKYRAIGMVSARMDDVANYLPARLSWLLLGIAAGLCRLSGWRALRIGWRDRYNHSSPNCAWSEACVAGALGIQLGGPNNYFGERVDKPWIGDAQRDISVDDISRTIRLMWVASTLALALLIAARCGLSGVA

SEQ ID NO: 188 - protein, 300 aa, Salmonella typhimurium
MRAHYSYLKGDNVAVAQCPASCGELIQGWILGSEKLVSCPVDWYSTVAVTAAPPLINERPLSRAMVERVLAHWQYPAHWSNEIRVDVRSSIPVAKGMASSTADIAATAVATAHHLGHSLDETTLAQLCVSIEPTDSTVFHQLTLFDHNNAATQIACEPPPPIDLLVLESPVTLRTQDYHRLPRQQKLIASSATLQQAWNLVQEACITQNPLRLGEAATLSAIASQTLLPKPGFTALLSLVEECDLYGLNVAHSGSVVGLMLDRKRHDIARLKGKLAEKKLTRHWPKQHLLKMVTGGVKLQ

SEQ ID NO: 189 - protein, 93 aa, Salmonella typhimurium
MKKTLMLLAMVVALVILPFFINHGGEYGGSDGEAESQIQAIAPQYKPWFQPLYEPASGEIESLLFTLQGSLGAAVIFYILGYCKGKQRRDDRA

SEQ ID NO: 190 - protein, 225 aa, Salmonella typhimurium
MTGLDRLSYQSRWAHVAPQRKFLLWLAMMILAFVLPPVGQGIELLIIAGLSCWLLRISLWRWCRWMAIPFGFLLVGVITIIFSISREPQMLLAGISVGPYWIGITRAGVVTANETFWRSLTALSATLWLVMNLPFPQLISLLKRAHIPRLLTEQILLTWRFLFILLDEAVAIRRAQTLRFGYCSLPNGYRSLAMLAGLLFTRVLMRYQQMTTTLDIKLYQGDFHL

SEQ ID NO: 191 - protein, 245 aa, Salmonella typhimurium
MKLEQQLRQLSFSGLAAALLLMVVPQQAFAMHIMEGFLPPVWALAWWLLFLPCLWYGLVRLRRIVQEDNHQKVLLALCGAFIFVLSALKIPSVTGSCSHPTGVGLAVILFGPGVVAILGAVVLLFQALLLAHGGLTTLGANGMSMAVIGPVVGYLVWKMACRAGLRRDVAVFLCAMLADLATYFVTSVQLGVAFPDPHAGATGSVVKFMGIFCLTQIPVAIAEGLLTVMIYDQLTKRQVITVQGH

SEQ ID NO: 192 - protein, 271 aa, Salmonella typhimurium
MLATSDLWFRYQNEPVLKGLNMDFSLSPVTGLVGANGCGKSTLFMNLSGLLRPQKGAVLWQGKPLDYSKRGLLALRQQVATVFQDPEQQIFYTDIDSDIAFSLRNLGVPEAEITRRVDEALTLVDAQHFRHQPIQCLSHGQKKRVAIAGALVLQARYLLLDEPTAGLDPAGRTQMIAIIRRIVAQGNHVIISSHDIDLIYEISDAVYVLRQGQILTHGAPGEVFACTEAMEHAGLTQPWLVKLHTQLGLPLCKTETEFFHRMQKCAFREAS

SEQ ID NO: 193 - skipped

SEQ ID NO: 194 - protein, 616 aa, Escherichia coli
MPKYRSATTTHGRNMAGARALWRATGMTDADFGKPIIAVVNSFTQFVPGHVHLRDLGKLVAEQIEAAGGVAKEFNTIAVDDGIAMGHGGMLYSLPSRELIADSVEYMVNAHCADAMVCISNCDKITPGMLMASLRLNIPVIFVSGGPMEAGKTKLSDQIIKLDLVDAMIQGADPKVSDSQSDQVERSACPTCGSCSGMFTANSMNCLTEALGLSQPGNGSLLATHADRKQLFLNAGKRIVELTKRYYEQNDESALPRNIASKAAFENAMTLDIAMGGSTNTVLHLLAAAQEAEIDFTMSDIDKLSRKVPQLCKVAPSTQKYHMEDVHRAGGVIGILGELDRAGLLNRDVKNVLGLTLPQTLEQYDVMLTQDDAVKNMFRAGPAGIRTTQAFSQDCRWDTLDDDRANGCIRSLEHAYSKDGGLAVLYGNFAENGCIVKTAGVDDSILKFTGPAKVYESQDDAVEAILGGKVVAGDVVVIRYEGPKGGPGMQEMLYPTSFLKSMGLGKACALITDGRFSGGTSGLSIGHVSPEAASGGSIGLIEDGDLIAIDIPNRGIQLQVSDAELAARREAQDARGDKAWTPKNRERQVSFALRAYASLATSADKGAVRDKSKLGG

SEQ ID NO: 195 - protein, 562 aa, Acidobacterium capsulatum
MSSEQNGSAKKNSVALTEGPNRAPARAMLRAIGFTRDDLRKPIIGIANTWTEIGPCNFHLRQIAEAVKQGIREAGGTPMEFNTVTISDGITMGTEGMKASLVSREVIADSIELVTRGNLFDGLVCIAGCDKNLPGTVMALARLDVPGLMLYGGSIAPGHLNGKDLTVQDVFEAVGSHAAGKLDDAGLEAVEANACPGAGACGGQFTANTMAMACEFMGISPMSISGVPAFLPEKLASAKEAGKLVLELAKRDLRPSQLITKESLENAIASVAASGGSTNAVLHFLAIAHERGIALSIDDFDRISEKTPLLCDMKPGGKYVAADYQKAGGSRLLAKRLIEGGYIHSDCLNASGRTLAEEAALAVEEPGQKVIFPATNALKPTGGLVILKGNLAPEGCVIKVAGHSRLTHRGPARVFDREQDAFAAVENGSIQPDDVMVIRYEGPKGGPGMREMLGVTAAIAGIPALSETVALLTDGRFSGATRGLMAGHVSPEAAVRGPIAAVRNGDMIHFDIPNRRLDVELTDAQIEERLKDWKEPAPHFSRGVFKKYVDTVSSAARGAVTN

SEQ ID NO: 196 - protein, 585 aa, Saccharomyces cerevisiae
MGLLTKVATSRQFSTTRCVAKKLNKYSYIITEPKGQGASQAMLYATGFKKEDFKKPQVGVGSCWWSGNPCNMHLLDLNNRCSQSIEKAGLKAMQFNTIGVSDGISMGTKGMRYSLQSREIIADSFETIMMAQHYDANIAIPSCDKNMPGVMMAMGRHNRPSIMVYGGTILPGHPTCGSSKISKNIDIVSAFQSYGEYISKQFTEEEREDVVEHACPGPGSCGGMYTANTMASAAEVLGLTIPNSSSFPAVSKEKLAECDNIGEYIKKTMELGILPRDILTKEAFENAITYVVATGGSTNAVLHLVAVAHSAGVKLSPDDFQRISDTTPLIGDFKPSGKYVMADLINVGGTQSVIKYLYENNMLHGNTMTVTGDTLAERAKKAPSLPEGQEIIKPLSHPIKANGHLQILYGSLAPGGAVGKITGKEGTYFKGRARVFEEEGAFIEALERGEIKKGEKTVVVIRYEGPRGAPGMPEMLKPSSALMGYGLGKDVALLTDGRFSGGSHGFLIGHIVPEAAEGGPIGLVRDGDEIIIDADNNKIDLLVSDKEMAQRKQSWVAPPPRYTRGTLSKYAKLVSNASNGCVLDA

SEQ ID NO: 197 - protein, 555 aa, Aquifex aeolicus
MKFRSDKVKKGIERAPHRALLRACGLSDEDFDKPLIGIANSYIDIIPGHVHLREFVEPIKEEVRKLGGVPIEFNVIGVDDGIAMGHEGMHYSLPSRELIADSIETVVNAHQLDALICIPNCDKIVPGMLMGALRVNVPTVFISGGPMLAGEVNGQKVDLISVFEGIGKVKRGEISEQELKVIEASACPTCGSCSGMFTANSMNCLTEVLGLALPGNGTILAIDPRREILARNAVKALFELLEKDVKPRDIVTEEALDDAFTVDIAMGGSSNTILHLLAIAREAGIEYNLAKINEISKRTPTICKISPASHYHIEDLDRVGGIPTIMKELSKLGLLHTERKTVSGKTIGEIISDAPDADGEVVRTIENPYSKDGGIAILFGNLAPEGAVVKTAGVDPKMLTFKGKAICFDSEEEAIEGILGGKVKPGHVVVIRYEGPKGGPGMREMLSPTSAIMGMGLGDKVALITDGRFSGGTRGACVGHISPEAAAGGPIGIVKDGDEILIDIPNRRIELLISEEEFNERMKNFKPKQKEIKSSWLRRYAKLVTSASKGAILEA

SEQ ID NO: 198 - protein, 558 aa, Bacillus subtilis
MAELRSNMITQGIDRAPHRSLLRAAGVKEEDFGKPFIAVCNSYIDIVPGHVHLQEFGKIVKEAIREAGGVPFEFNTIGVDDGIAMGHIGMRYSLPSREIIADSVETVVSAHWFDGMVCIPNCDKITPGMLMAAMRINIPTIFVSGGPMAAGRTSDGRKISLSSVFEGVGAYQAGKINENELQELEQFGCPTCGSCSGMFTANSMNCLSEALGLALPGNGTILATSPERKEFVRKSAAQLMETIRKDIKPRDIVTVKAIDNAFALDMALGGSTNTVLHTLALANEAGVEYSLERINEVAERVPHLAKLAPASDVFIEDLHEAGGVSAALNELSKKEGALHLDALTVTGKTLGETIAGHEVKDYDVIHPLDQPFTEKGGLAVLFGNLAPDGAIIKTGGVQNGITRHEGPAVVFDSQDEALDGIINRKVKEGDVVIIRYEGPKGGPGMPEMLAPTSQIVGMGLGPKVALITDGRFSGASRGLSIGHVSPEAAEGGPLAFVENGDHIIVDIEKRILDVQVPEEEWEKRKANWKGFEPKVKTGYLARYSKLVTSANTGGIMKI

SEQ ID NO: 199 - protein, 613 aa, Corynebacterium glutamicum
MIPLRSKVTTVGRNAAGARALWRATGTKENEFGKPIVAIVNSYTQFVPGHVHLKNVGDIVADAVRKAGGVPKEFNTIAVDDGIAMGHGGMLYSLPSREIIADSVEYMVNAHTADAMVCISNCDKITPGMLNAAMRLNIPVVFVSGGPMEAGKAVVVDGVAHAPTDLITAISASASDAVDDAGLAAVEASACPTCGSCSGMFTANSMNCLTEALGLSLPGNGSTLATHAARRALFEKAGETVVELCRRYYGEEDESVLPRGIATKKAFENAMALDMAMGGSTNTILHILAAAQEGEVDFDLADIDELSKNVPCLSKVAPNSDYHMEDVHRAGGIPALLGELNRGGLLNKDVHSVHSNDLEGWLDDWDIRSGKTTEVATELFHAAPGGIRTTEAFSTENRWDELDTDAAKGCIRDVEHAYTADGGLVVLRGNISPDGAVIKSAGIEEELWNFTGPARVVESQEEAVSVILTKTIQAGEVLVVRYEGPSGGPGMQEMLHPTAFLKGSGLGKKCALITDGRFSGGSSGLSIGHVSPEAAHGGVIGLIENGDIVSIDVHNRKLEVQVSDEELQRRRDAMNASEKPWQPVNRNRVVTKALRAYAKMATSADKGAVRQVD

SEQ ID NO: 200 - protein, 607 aa, Deinococcus radiodurans
MPTYRSRTTTEGRNMAGARALWRATGMTDGDFQKPIIAVVNSFTQFVPGHVHLKDLGQLVAGEIAAAGGVAKEFNTIAVDDGIAMGHDGMLYSLPSREIIADSVEYMVNAHCADAMVCISNCDKITPGMLMAALRLNIPTVFVSGGPMEAGKVKLGDGEHALDLVDAMVMAADDSVSEEDIELVERSACPTCGSCSGMFTANSMNCLTEALGLSLPGNGSVLATHSDRQNLFKRAGHLIVDLAKRYYEGDDESVLPRSVATYDAFENAMTLDISMGGSTNTVLHLLAAAHEAGVDFTMQDIDQLSRRVPVLCKVAPAKNDVHMEDVHRAGGIMGILGELDRAGLLKTDVSTVHAPSMSAALNEWDVVRNENHHEFYRAAPGGIPTVFAFSQSRRYRELDTDREAGVIRSAEHAFSQDGGLAVLYGNLAEDGCIVKTAGVDESILKFSGPARVFESQDASVDAILSGEVKEGEVVLIRYEGPRGGPGMQEMLYPTSYLKSKGLGKACALVTDGRFSGGSSGLSIGHVSPEAAEGGTIGLVETGDLIEIDIPNRTIHLAVPDAELAARRQKREEEGWHPAEPRPRKITAALRAYASMTTSASRGAVRNI

SEQ ID NO: 201 - protein, 550 aa, Methanococcus maripaludis
MISDNVKKGVIRTPNRALLKACGYTDEDMEKPFIGIVNSFTEVVPGHIHLRTLSEAAKHGVYANGGTPFEFNTIGICDGIAMGHEGMKYSLPSREIIADAVESMARAHGFDGLVLIPTCDKIVPGMIMGALRLNIPFIVVTGGPMLPGEFQGKKYELISLFEGVGEYQVGKITEEELKCIEDCACSGAGSCAGLYTANSMACLTEALGLSLPMCATTHAVDAQKVRLAKKSGSKIVDMVKEDLKPTDILTKEAFENAILVDLALGGSTNTTLHIPAIANEIENKFITLDDFDRLSDEVPHIASIKPGGEHYMIDLHNAGGIPAVLNVLKEKIRDTKTVDGRSILEIAESVKYINYDVIRKVEAPVHETAGLRVLKGNLAPNGCVVKIGAVHPKMYKHDGPAKVYNSEDEAISAILGGKIVEGDVIVIRYEGPSGGPGMREMLSPTSAICGMGLDDSVALITDGRFSGGSRGPCIGHVSPEAAAGGVIAAIENGDIIKIDMIEKEINVDLDESVIKERLSKLGEFEPKIKKGYLSRYSKLVSSADEGAVLK

SEQ ID NO: 202 - protein, 613 aa, Pseudomonas putida
MPDYRSKTSTQGRNMAGARALWRATGMKDEDFKKPIIAIANSFTQFVPGHVHLKDLGQLVAREIERAGGVAKEFNTIAVDDGIAMGHDGMLYSLPSREIIADAVEYMVNAHCADAIVCISNCDKITPGMLMAALRLNIPVIFVSGGPMEAGKTKLASHGLDLVDAMVIAADSSASDEKVAEYERSACPTCGSCSGMFTANSMNCLTEALGLALPGNGSTLATHADREQLFLTAGRTIVELCKRYYGENDESVLPRSIANFKAFENAMMLDIAMGGSTNTILHLLAAAQEGEVEFDLRDIDRLSRKVPQLCKVAPNIQKYHMEDVHRAGGIFSILGSLARGGLLHTDLPTVHSRSMEEAIAKWDITQTDDEAVHTFFKAGPAGIPTQTAFSQSTRWETLDDDRENGCIRSFEHAYSQEGGLAVLYGNIALDGCVVKTAGVDESIHVFEGNAKIFESQDSAVRGILADEVKAGDIVIIRYEGPKGGPGMQEMLYPTSYLKSKGLGKACALLTDGRFSGGTSGLSIGHASPEAAAGGAIGLVRDGDKVLIDIPNRSINLLVSDEELAERRVEQDKKGWKPAEVRPRKVTTALKAYALLATSADKGAVRNKAMLEGL

SEQ ID NO: 203 - protein, 619 aa, Synechococcus elongatus
MPQYRSRTTTYGRNMAGARALWRATGMKDEDFEKPIIAVANSFTQFVPGHVHLKDLGQLVAREIERAGGVAKEFNTIAVDDGIAMGHGGMLYSLPSRDLIADSVEYMVNAHCADALVCISNCDKITPGMLMAALRLNIPAVFVSGGPMEAGKVILNGEERHLDLVDAMVVAADDRESDEDVATIERSACPTCGSCSGMFTANSMNCLTEALGLSLPGNGSLLATHGDRKELFLEAGRLAVKLAKQYYEQDDESVLPRSIASFKAFENAICLDIAMGGSTNTVLHLLAAAHEAGVDFTMKDIDRLSRKIPNLCKVAPSTQKYHMEDVHRAGGVIAILGELDRAGLLHREVPTVHSPSLGAALDQWDINRETATEEAKSRYLAAPGGVPTQEAFSQSKRWTALDLDRENGCIRDIEHAYSQDGGLAVLYGNLAEQGCIVKTAGVDENILVFSGPAVVCESQDEAVNWILNGRVKEGDVVLIRYEGPRGGPGMQEMLYPTSYLKSKGLGKACALITDGRFSGGTSGLSIGHVSPEAAEGGLIALVEQGDRIEIDIPNRRIHLAVSEEELAHRRAAMEARGDQAWTPKDRDRPISQALQAYAAMTTSAARGGVRDLSQILGSR

SEQ ID NO: 204 - protein, 554 aa, Thermotoga maritima; identical to SEQ ID NO: 141

SEQ ID NO: 205 - protein, 264 aa, Escherichia coli
MKPTTISLLQKYKQEKKRFATITAYDYSFAKLFADEGLNVMLVGDSLGMTVQGHDSTLPVTVADIAYHTAAVRRGAPNCLLLADLPFMAYATPEQAFENAATVMRAGANMVKIEGGEWLVETVQMLTERAVPVCGHLGLTPQSVNIFGGYKVQGRGDEAGDQLLSDALALEAAGAQLLVLECVPVELAKRITEALAIPVIGIGAGNVTDGQILVMHDAFGITGGHIPKFAKNFLAETGDIRAAVRQYMAEVESGVYPGEEHSFH

SEQ ID NO: 206 - protein, 303 aa, Escherichia coli
MKITVLGCGALGQLWLTALCKQGHEVQGWLRVPQPYCSVNLVETDGSIFNESLTANDPDFLATSDLLLVTLKAWQVSDAVKSLASTLPVTTPILLIHNGMGTIEELQNIQQPLLMGTTTHAARRDGNVIIHVANGITHIGPARQQDGDYSYLADILQTVLPDVAWHNNIRAELWRKLAVNCVINPLTAIWNCPNGELRHHPQEIMQICEEVAAVIEREGHHTSAEDLRDYVMQVIDATAENISSMLQDIRALRHTEIDYINGFLLRRARAHGIAVPENTRLFEMVKRKESEYERIGTGLPRPW

SEQ ID NO: 207 - protein, 283 aa, Escherichia coli
MLIIETLPLLRQQIRRLRMEGKRVALVPTMGNLHDGHMKLVDEAKARADVVVVSIFVNPMQFDRPEDLARYPRTLQEDCEKLNKRKVDLVFAPSVKEIYPNGTETHTYVDVPGLSTMLEGASRPGHFRGVSTIVSKLFNLVQPDIACFGEKDFQQLALIRKMVADMGFDIEIVGVPIMRAKDGLALSSRNGYLTAEQRKIAPGLYKVLSSIADKLQAGERDLDEIITIAGQELNEKGFRADDIQIRDADTLLEVSETSKRAVILVAAWLGDARLIDNKMVELA

(19) United States
(12) Patent Application Publication
(10) Pub. No.: US 2024/0368659 A1
(43) Pub. Date: Nov. 7, 2024

(54) MICROBIAL CELL FACTORIES PRODUCING VITAMIN B COMPOUNDS

(71) Applicant: BIOSYNTIA APS, Copenhagen (DE)

(72) Inventors: Carlos Guillermo ACEVEDO-ROCHA, Copenhagen (DK); Daniel Boer BONIFACIO, Copenhagen (DK); Benjamin Joseph CADDELL, Copenhagen (DK); Hans Jasper GENEE, Copenhagen (DK); Luisa Simona GRONENBERG, Copenhagen (DK); Kathleen Frances SMART, Copenhagen (DK); Nils MYLING-PETERSEN, Copenhagen (DK); Bo SALOMONSEN, Copenhagen (DK); Gijs VERKLEIJ, Copenhagen (DK)

(21) Appl. No.: 18/572,531

(22) PCT Filed: Jul. 14, 2022
(86) PCT No.: PCT/EP2022/069711

§ 371 Date: Dec. 20, 2023

(30) Foreign Application Priority Data
Jul. 16, 2021 (EP) 21186217.2
Apr. 12, 2022 (EP) 22167864.2

(51) Int. Cl. C12P 19/42; C12N 9/12; C12N 9/16; C12N 15/70; C12P 17/16

(52) U.S. Cl. CPC C12P 19/42; C12N 9/12; C12N 9/16; C12N 15/70; C12P 17/167; C12Y 207/11011; C12Y 301/04053; C12N 2800/101

(57) ABSTRACT

The present disclosure relates to a genetically modified host cell having increased production of one or more vitamin B compounds, wherein the host cell is genetically modified by mutating one or more native polynucleotide constructs for reducing formation of a CRP-cAMP complex in the host cell and/or introducing one or more genetic alterations increasing the degradation and/or non-CRP binding of cAMP in the host cell; whereby the production of the vitamin B compound in the genetically modified host cell is increased compared to a parent host cell.

### FIELD OF THE INVENTION

The present invention relates to genetically modified host cells having increased production of vitamin B compounds through providing for a reduced and/or eliminated binding of cAMP to cAMP receptor protein (CRP); to mutants of native genes and encoded polypeptides providing for a reduced binding of cAMP to CRP; to genetic constructs for expression of such mutants; to cultures of the genetically modified host cells and its use to produce vitamin B compounds; to fermentation liquids comprising vitamin B compounds resulting from such production; to compositions comprising the fermentation liquid; to dietary or pharmaceutical preparations made from such compositions and to the uses of such compositions and preparations.

### BACKGROUND OF THE INVENTION

Vitamin B compounds, such as biotin (B₇) are in nature produced by some microbes and plants. The biosynthesis pathways for vitamin B compounds in natural organisms are generally well described in the art. For example, in nature, biotin is synthesized by a linear pathway involving the fatty acid biosynthetic pathway (see FIG. 6). The initial substrate of biotin synthesis in Escherichia coli (E. coli) is malonyl-ACP, which is also the starting metabolite for fatty acid synthesis. Prior to entering the fatty acid cycle, malonyl-ACP is masked by the SAM (S-adenosylmethionine)-dependent methyltransferase, BioC, thereby generating a malonyl-ACP methyl ester. Subsequently, two rounds of fatty acid chain elongation yield the molecule pimeloyl-ester-ACP. Hydrolysis of the O-methyl group of the pimeloyl-ester-ACP by a dedicated esterase, BioH, allows this molecule to exit the fatty acid elongation cycle. Subsequently, the intermediate, pimeloyl-ester-ACP, is converted to biotin via a biotin-specific pathway. In this pathway, BioF catalyzes the PLP-dependent decarboxylative aldol condensation of pimeloyl-ACP with alanine to yield KAPA (8-Amino-7-oxononanoate). BioA (and BioK) catalyzes the PLP-dependent transamination of KAPA to yield DAPA (7,8-diaminopelargonate), where the donor is SAM; with the by-product S-adenosyl-oxomethionine. BioD catalyzes the ATP-driven carboxylation and ring closure of DAPA to form the thiophane ring in desthiobiotin (DTB). The final step in the biotin synthesis pathway is one of the most complex biological reactions known, since it involves the introduction of a sulfur bridge between two hydrocarbons by biotin synthase (BioB), to yield biotin.

Further, thiamine (B₁) is in nature produced by some microbes and plants. The biosynthesis pathways for thiamine in natural organisms are generally well described in the art. For example, in nature, thiamine is synthesized by the pathway shown in FIG. 7.

The use of microorganism-based cell factories is a potential route for the biosynthetic production of B vitamins (Acevedo-Rocha, et al. 2019). The advantages of a recombinant microorganisms such as E. coli as a cell factory for production of bio-products are widely recognized due to the fact that: (i) it has unparalleled fast growth kinetics; with a doubling time of about 20 minutes when cultivated in glucose-salts media and under optimal environmental conditions, (ii) it easily achieves a high cell density; where the theoretical density limit of an E. coli liquid culture is estimated to be about 200 g dry cell weight/L or roughly 1×10\hat{ }13 viable bacteria/mL. Additionally, there are many molecular tools and protocols at hand for genetic modification of E. coli; as well as it being an organism that is amenable to the expression of heterologous proteins; both of which may be essential for obtaining high-level production of desired bio-products.

Cellular production capacity of vitamin B compounds is influenced by many factors and is to a large extent only sparsely described in the art. WO2019/012058 and WO2020/148351 discloses that mutating transcription factor IscR to favor the apoprotein improves conversion of for example desthiobiotin to biotin by activating the SUF and ISC operons producing iron-sulfur cluster (FeS) clusters needed by biotin synthase (BioB) and/or favor the apoprotein improves conversion of for example 5-aminoimidazole ribotide (AIR) to thiamine by activating the SUF and ISC operons producing iron-sulfur cluster (FeS) clusters needed by phosphomethylpyrimidine synthase (ThiC).

Further Ifuku et al. (1995) discloses that BioB over-expression can induce cell death in E. coli strains, possibly due to depletion of FeS clusters from the cell and/or the generation of Reactive Oxygen Species (ROS).

Another factor affecting production of vitamin B compounds in cells is the metabolic state of the cell. There are several cellular regulator proteins that have large effects on the metabolic state of the cell. One such protein is the transcription factor cAMP receptor protein (CRP), also known as catabolite gene activator protein (CAP) which regulates expression of hundreds of genes in E. coli when bound to small molecule adenosine 3′,5′-cyclic monophosphate or cyclic AMP (cAMP, CAS Number 60-92-4). cAMP is synthesized by adenylate cyclase from adenosine triphosphate (ATP).

Deutscher et al. (2006) discloses that when glucose supply is depleted, while other carbon sources are available, the phosphotransferase system (PTS) proteins become phosphorylated, resulting in activation of adenylate cyclase (CyaA) by enzyme IIA-Glc (CRR) and thus cAMP synthesis, ultimately leading to an active CRP-cAMP complex. When glucose is in excess PTS remains unphosphorylated ultimately leading to low cAMP and low levels of CRP-cAMP complex. Shimada et al. (2011) discloses that CRP-cAMP activates (among others) genes involved in the utilization of secondary carbon sources, as well as the transcription of PTS/glucose import genes. Shimada et al. (2011) further discloses that an estimated ˜400-500 operons are under direct control of the CRP-cAMP complex including the following processes: a) carbon metabolism (glycolysis and gluconeogenesis); b) aerobic respiration (TCA cycle); c) nitrogen metabolism; d) regulatory roles (control of many transcription factors); and e) stress response. Barth et al (2009) discloses that a cellular decrease of cAMP derepresses rpoS, a gene that encodes the alternative sigma factor σS, which activates the general stress response in E. coli. Basak & Jiang (2012) discloses that specific mutations to the active site of CRP enables E. coli to tolerate ROS due to expression of stress response genes. Perrenoud & Sauer (2005) showed that deletion of CyaA influences biomass yields, growth rates, glucose consumption rate, acetate production, phosphoenolpyruvate levels and serine levels, while no influence on TCA cycle flux were observed.

Further background art includes US2006/121558 which discloses that mutation of CyaA impacts on cellular production of carotenoids in E. coli; WO2011102305 which discloses that mutation of CyaA impacts on cellular production of amino acids in Enterobacteriaceae; US20140096439 which discloses that mutation of CyaA impacts on cellular production of butanol in yeast; AU2012202630 which discloses that mutation of CyaA impacts on cellular production of isoprenoids in E. coli; WO2013003432 discloses that mutation of CyaA impacts on cellular production of succinate in E. coli; U.S. Pat. No. 9,932,598 which discloses that mutation of CyaA impacts on cellular production of carbohydrates in E. coli; EP3050970 which discloses that mutation of CyaA impacts on cellular production of 1,4-butanediol in E. coli and other organisms; US20180100169 which discloses that mutation of CyaA impacts on cellular production of 2,4-dihydroxybutyrate in E. coli. WO2021122687 which states that increased cyclic AMP levels and mutations causing this increase the production of fine chemicals, especially human milk oligosaccharides, in host cells.

### SUMMARY OF THE INVENTION

Against this background art, the inventors of the present invention have now found that host cells producing vitamin B compounds genetically modified to reduce binding of cAMP to CRP unexpectedly have increased production of vitamin B compounds. The present inventors have also identified several ways to reduce the binding of cAMP to CRP (see FIGS. 1, 8 and 9), including deleting CRP and/or eliminating and/or decreasing the ability of the cell to synthesize cAMP as well as degrading cAMP in vivo. It is contemplated that the increased production of vitamin B compounds arise out of increased expression and/or activity of key enzymes in the pathways producing the vitamin B compounds, such as BioB for biotin (vitamin B₇), ThiC for thiamine (vitamin B₁), NadA for quinolate (vitamin B₃), IlvD for pantothenate (vitamin B₅) and CobG for cobalamin (vitamin B₁₂). Pathways for vitamin B₃ complex (NR, NAM, and NA), cobalamin (vitamin B₁₂) and pantothenate (vitamin B₅) are shown in FIGS. 10, 11 and 12 respectively. All these enzymes are dependent on iron sulfur (FeS) clusters and are known to be bottlenecks of their biosynthetic pathways. Moreover, it is contemplated that the increased production of thiamines arise out of increased expression and/or activity of key enzymes in the pathways producing the thiamine, TMP or TPP, such as ThiC, ThiD, ThiM, THiE, ThiF, ThiS, ThiG, Thil, ThiH or ThiO. Specifically, for thiamine, herein illustrated for example by ThiC it has been found that mutations that eliminate or decrease the amount of CRP-cAMP complex increase the in vivo activity of overexpressed pathway enzymes, such as phosphomethylpyrimidine synthase (ThiC) that produces the pyrimidine part of thiamine HMP-P, which can be phosphorylated by ThiD and then ligated with THZ-P (thiazole-phosphate) by ThiE (THZ can be added to the media and phosphorylated by ThiM) to produce TMP (thiamine monophosphate), which can be dephosphorylated by an heterologous phosphatase (AtTH2), thereby increasing production of thiamine, TMP or TPP from glucose in microorganism-based cell factories.

Specifically, for vitamin B compounds, herein illustrated for example by biotin (B₇) or thiamine (B₁), it has been found that mutations that eliminate or decrease the amount of CRP-cyclic AMP complex increase the in vivo activity of overexpressed pathway enzymes, such as biotin synthase (BioB) or phosphomethylpyrimidine synthase (ThiC), and thereby increase production of vitamin B compounds such as biotin from glucose in microorganism-based cell factories. Several genes are directly related to regulating or catalyzing the formation of the CRP-cyclic AMP complex and therefore there are several distinct genes that can be modified to arrive at the same phenotype of an improved vitamin B compound production strain. The regulatory effect of eliminating or decreasing the amount of CRP-cyclic AMP complex in the cell is very complex and is known to increase the yield of cell biomass produced from glucose; however, the improvement in vitamin B compound production in genetically modified cell factories has surprisingly been found to exceed the increase in biomass, i.e. production of vitamin B compound, such as biotin or thiamine, per gram of biomass is improved on top of the increase in biomass. While the exact mechanism for improvement of pathway enzymes for vitamin B compounds (e.g., BioB or ThiC) is unknown, it is believed that the change in metabolism brought about by the lack of cyclic AMP complex is beneficial for the formation of functional FeS loaded enzymes, the bottleneck for many enzymatic steps, such as BioB, ThiC, NadA, IlvD and CobG producing biotin, thiamine, quinolate, pantothenate and cobalamin respectively.

CRP is natively activated as a transcriptional regulator by binding to its allosteric activator cyclic AMP (cAMP), which is produced by the enzyme adenylate cyclase (encoded by CyaA) (FIG. 1). Under low-glucose conditions, adenylate cyclase is activated by the phosphorylated state of glucose-specific enzyme IIA or EIIA (encoded by CRR). Under high glucose conditions EIIA is not phosphorylated and does not activate adenylate cyclase, decreasing production of cyclic AMP and therefore of the active CRP-cAMP complex. When CRR is deleted, it no longer activates adenylate cyclase. When adenylate cyclase is mutated or deleted, cAMP cannot be formed in the cell and when CRP is mutated or deleted, it can no longer bind to cAMP. In all cases the CRP-cAMP complex is decreased or eliminated. Cellular cAMP levels can also be decreased by overexpressing enzymes that degrade cAMP such as cAMP-phosphodiesterase CpdA and cAMP deaminase CadD.

Accordingly, in a first aspect the invention provides a genetically modified host cell having increased production of one or more vitamin B compound, wherein the host cell is genetically modified by
a) mutating or deleting one or more native polynucleotide constructs for reducing or eliminating formation of a CRP-cAMP complex in the host cell and/or
b) introducing one or more genetic alterations increasing the degradation of cAMP and/or binding of cAMP by a polypeptide which is not CRP in the host cell;
   whereby the production of the vitamin B compound in the genetically modified host cell is increased compared to a parent host cell.

In a further aspect the invention provides for mutated polypeptides which are at least 90% identical to
a) the mutant CRP having a sequence comprised in SEQ ID NO: 39 and further comprising one or more mutations in positions corresponding to T12, D138, T146, F69, R82, V139, G57, K58, E59, M60, 161, L62, S63, G72, E73, L74, R83, S84, T128, S129, A136, F137, Q171, E172, 1173, G174, Q175, 1176, V177, G178, C179, S180, R181, E182, T183, V184, G185, and/or R186 of SEQ ID NO: 39;
b) the mutant CRR having a sequence comprised in SEQ ID NO: 41 and further comprising one or more mutations in positions corresponding to H76 and/or H91 of SEQ ID NO: 41;
c) the mutant CyaA having a sequence comprised in SEQ ID NO: 43 and further comprising one or more mutations in positions corresponding to G60, K59, L63, T65, R188, G195, K196, R192, S103, S113, D114, D116, W118, E185, T189, K260, K264, K332, W200, and/or D300 of SEQ ID NO: 43.

In a still further aspect, the invention provides a polynucleotide construct comprising a mutated polynucleotide sequence of the invention encoding a cAMP receptor protein (CRP), a carbohydrate repression resistance protein (CRR) or a adenylate cyclase protein (CyaA) operably linked to one or more control sequences.

In a still further aspect, the invention provides a cell culture, comprising the genetically modified host cell of the invention and a growth medium.

In a still further aspect, the invention provides a genetically modified host cell comprising the nucleic acid construct.

In a still further aspect, the invention provides a cell culture, comprising the genetically modified host cell of the invention and a growth medium.

In a still further aspect, the invention provides a method for producing a vitamin B compound comprising:
d) culturing the cell culture of the invention at conditions allowing the host cells to produce the vitamin B compound; and
e) optionally recovering and/or isolating the vitamin B compound.

In a still further aspect, the invention provides a fermentation composition comprising the cell culture of the invention and the vitamin B compound comprised therein.

In a still further aspect, the invention provides a composition comprising the fermentation composition of the invention and one or more carriers, agents, adjuvants, additives and/or excipients.

In a final aspect, the invention provides a kit of parts comprising:
a) the genetically modified cell of the invention and instructions for its use; and/or
b) the nucleic acid construct of the invention and instructions for use; and
c) optionally the cell to be modified.

### DESCRIPTION OF DRAWINGS AND FIGURES

The figures included herein are illustrative and simplified for clarity, and they merely show details which are essential to the understanding of the invention, while other details may have been left out. The figures and drawing include:

FIG. 1 shows a simplified model of the regulatory pathway of the transcriptional dual regulator CRP (encoded by CRP).

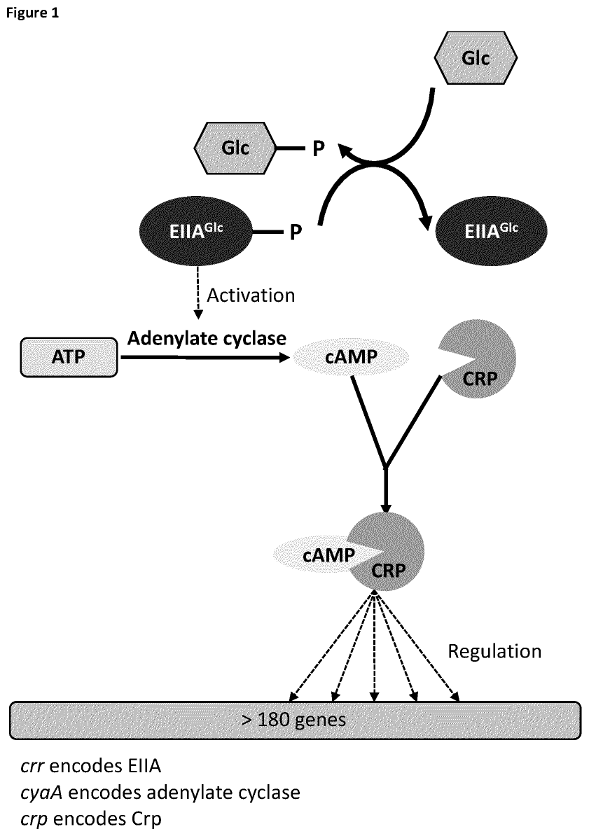

FIG. 2 shows growth of E coli strains with wild type or mutated CyaA on succinate. The growth is a phenotypic readout of functional formation of CRP-cAMP in E. coli. The wildtype strain shows growth on succinate (turbid, bottom) while a CyaA-mutant strain does not (clear, top).

FIG. 3 shows a bar diagram of biotin production from DTB of E. coli strains comprising an IPTG-inducible bioB expression plasmid and with inactivation of different genes in the chromosome.

FIG. 4 shows biotin yield per unit of biomass (mg biotin/g DCW) in fermentation with (BS2154) and without (BS3079) a functional CRP-cAMP complex.

FIG. 5 shows a bar graph of de novo biotin yield (mg/g glucose) and titre (mg/L) of E. coli strains in fed batch fermentation with (BS3304) and without (BS4759) a functional CRP-cAMP complex. Error bars represent standard deviation of triplicate conditions.

FIG. 6 shows the bacterial biotin production pathway from malonyl-CoA.

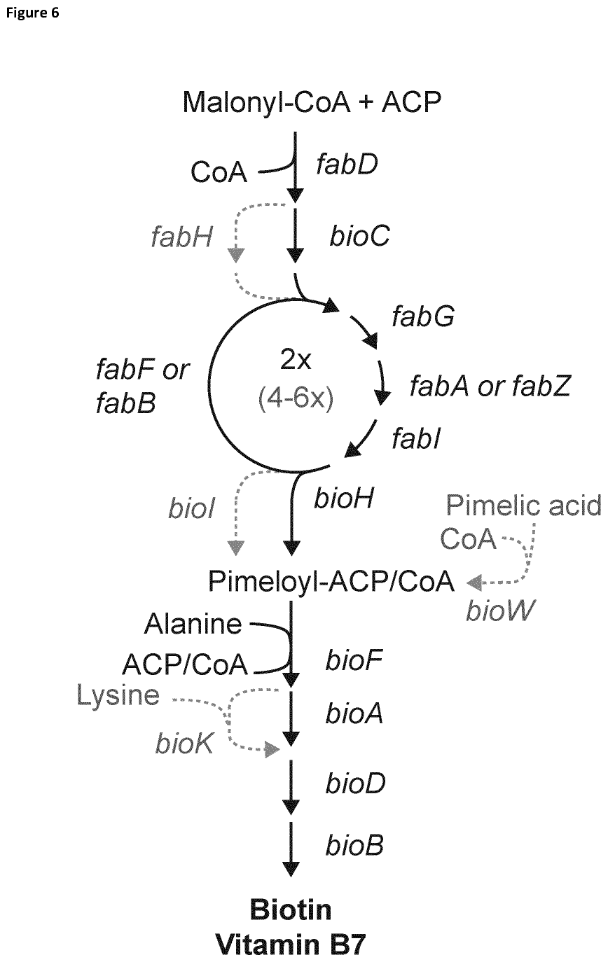

FIG. 7 shows the pathway for microbial production of thiamine in E. coli. In the pyrimidine branch (left, light gray), ThiC (phosphomethylpyrimidine synthase) converts its substrate AIR (aminoimidazole ribotide), that is available from the purine pathway, to HMP-P (hydroxymethylpyrimidine phosphate), which is subsequently phosphorylated to HMP-PP (hydroxymethylpyrimidine diphosphate) by ThiD (hydroxymethylpyrimidine/phosphomethylpyrimidine kinase). In the thiazole (THZ) branch (right, dark gray), the addition of THZ overcomes the need to express thiazole enzymes ThiFSGH because THZ can be taken up by cells and it can be phosphorylated to THZ-P [4-methyl-5-(beta-hydroxyethyl)thiazole monophosphate] by ThiM (hydroxyethylthiazole kinase). ThiE (thiamine-phosphate synthase) condenses both HMP-PP and THZ-P to form thiamine monophosphate (TMP), which is then dephosphorylated by a TMP-phosphatase to produce thiamine.

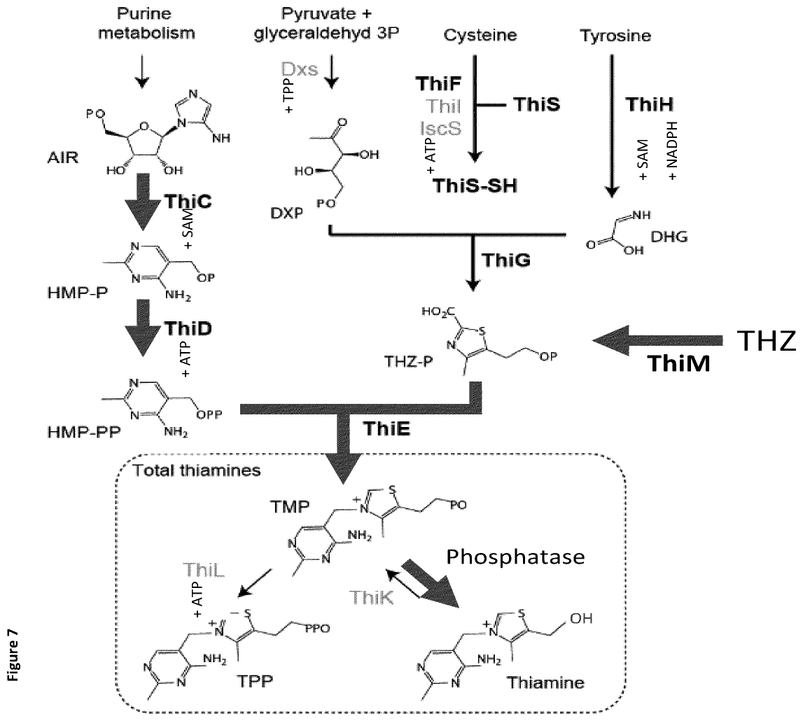

FIG. 8 shows a bar graph with thiamine production and growth (OD) after 24 h batch cultivation in mMOPS (supplemented with 500 μM THZ) of E. coli strains with IscR WT (BS04608) and mutant (BS04726) genotype, both with a functional CRP-cAMP complex, as well as IscR mutant strains without a functional CRP-cAMP complex caused by mutagenesis of CRP (BS04739) or cyaA (BS04786). Error bars represent standard deviation of quadruplicate conditions.

FIG. 9 shows a bar graph with thiamine production and growth (OD) after 24 h fed batch fermentations (supplemented with 500 μM THZ) of E. coli strains with IscR WT (BS04608) and mutant (BS04726) genotype, both with a functional CRP-cAMP complex, as well as IscR mutant strains without a functional CRP-cAMP complex caused by mutagenesis of CRP (BS04739) or cyaA (BS04786). Error bars represent standard deviation of quadruplicate conditions.

FIG. 10 shows the biosynthetic pathway of vitamin B₃ complex (NR, NAM, and NA). The intermediate quinolate is made by the condensation and cyclisation of 2-iminosuccinate by the [4Fe-4S] cluster enzyme NadA, requiring dihydroxy acetone phosphate (DHAP) as a co-factor. Nicotinic acid mononucleotide (NaMN) synthesis from quinolate is catalyzed by NadC and Nicotinic acid adenine dinucleotide (NaAD) formation from NaMN is catalyzed by NadD. Nicotinamide adenine dinulceotide (NAD+) formation is catalysed by NadE, via ATP-dependent amidation of NaAD. NudC and aphA subsequently convert NAD+ to nicotinamide mononucleotide (NMN) and Nicotinamide riboside (NR), respectively. NadE* is a NadE homologoue which prefers NaMN as a substrate and can convert NaMN to NMN directly. Nicotinamide (NAM) is produced by phosphatase activity of the NMN nucleosidase. NAM is converted to nicotinic acid by pncA. ATP=Adenosyl triphosphate; AMP=Adenosyl monophosphate; PP=Diphosphate; PRPP=5-phospho-alpha-D-ribose-1-diphosphate.

FIG. 11 shows the biosynthetic pathway for B₁₂ cobalamin. The synthesis of Precorrin-3-B from Precorrin-3-A is catalyzed by the FeS cluster enzyme CobG. The formation of Precorrin-8X needs 5 enzymes: CobJMFKL. The production of HBA and HBAD is then catalyzed by cobH and CobB, respectively. Cobalt in imported in the cell factory by using the cobal transporter made of the proteins CbiNQOM, which allows the formation of Adenosylcobyrate by the action of many enzymes: CobNSTPRQ. The final steps of the biosynthesis of adenoxyl-cobalamine via Adenosyl-cobinamide-P include CbiB and CobUSC. SAM=S-Adenosyl Methionine; SAH=S-Adenosyl-Homocysteine; ATP=Adenosyl Tri Phosphate; ADP=Adenosyl Bi Phosphate; HBA=Hydrogenobyrinic Acid; HBAD=Hydrogenobyrinic Acid a,c diamide; RAYP=(R)-1-amino-2-propanol O-2-phosphate; DMB=5,6-dimethylbenzimidazole; NDR=β-nicotinate D-ribonucleotide; R5P=α-ribazole 5′-phosphate; Nt=Nicotinate; GTP=Guanosyl Tri Phosphate; GMP=Guanosyl Mono Phosphate.

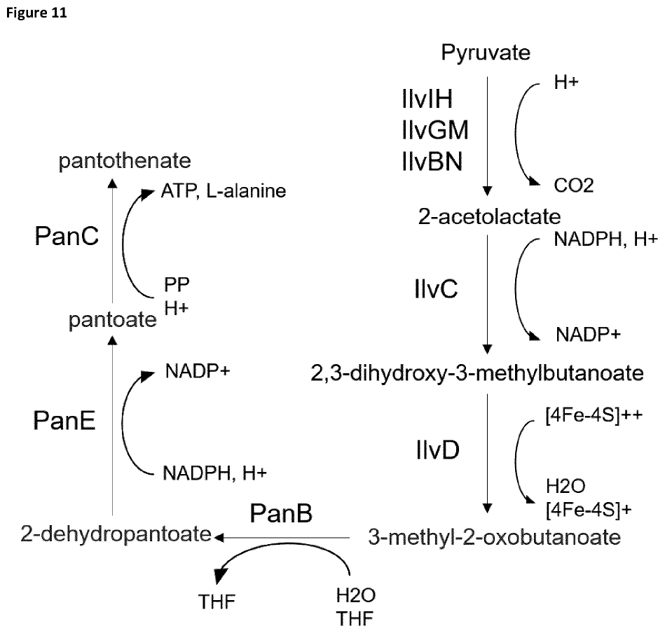

FIG. 12 shows the biosynthetic pathway for B₅ pantothenate. The first committed step for vitamin B₅ biosynthesis is the conversion of 3-methyl-2-oxobutanoate to 2-dehydropantoate by PanB (3-methyl-2-oxobutanoate hydroxymethyl-transferase). The substrate of PanB, 3-methyl-2-oxobutanoate, is produced by IlvD (Dihydroxy-acid dehydratase) from 2,3-dihydroxy-3-methylbutanoate. The product of PanB is 2-dehydropantoate, which is used by PanE (2-dehydropantoate 2-reductase) to produce pantoate. Finally, PanC (Pantothenate synthetase) converts pantoate to pantothenate. NADPH=Nicotinamide adenine dinucleotide phosphate. ATP=Adenosyl triphosphate; PP=Diphosphate.

### INCORPORATION BY REFERENCE

All publications, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILED DESCRIPTION OF THE INVENTION

Provided for herein are genetically modified host cells having increased production of one or more vitamin B compound, achieved by mutating one or more native genes for reducing formation of a CRP-cAMP complex in the host cell and/or introducing one or more genetic alterations increasing the degradation of cAMP and/or binding of cAMP by a polypeptide which is not CRP in the host cell; whereby the production of the vitamin B compound in the genetically modified host cell is increased compared to a parent host cell. For example, it was unexpectedly found that mutating CyaA and/or CRP and/or CRR in such a way as to delete, disrupt and/or attenuate the protein, e.g., produced a higher BioB or ThiC activity. It is not yet fully known if the cause of this effect is higher BioB or ThiC protein levels or higher BioB or ThiC activity per protein or a combination of both. Additionally, it was observed that deleting, disrupting and/or attenuating mutations in strains also provides for a higher production of the biotin precursor DTB or of thiamines including both thiamine and the precursor thiamine monophosphate (TMP), with the former being the most abundant (>98%) than the latter due to a very efficient phosphatase expression.

### Definitions

Any EC numbers used herein refers to Enzyme Nomenclature 1992 from NC-IUBMB, Academic Press, San Diego, California, including 30 supplements 1-5 published in Eur. J. Bio-chem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The nomenclature is regularly supplemented and updated; see e.g. http://enzyme.expasy.org/. The term “PEP” as used herein refers to phosphoenol pyruvate.

The term “CRP” as used herein refers to the cAMP receptor protein (CRP), a transcription factor which binds to cAMP and regulates transcription of a multitude of genes in many cells, such as araB, nadC, aceE, lacZ and many more.

The term “CyaA” as used herein refers to an adenylate cyclase protein (CyaA) converting ATP into cAMP.

The term “CRR” as used herein refers to an Enzyme IIA^{GIc} protein (also referred to as treD; gsr; iex; tgs; EIIA^{GIc}; Enzyme III^{GIc}; EIII^{GIc}; III^{GIc}; Enzyme IIA^{GIc}, CRR and Glucose-specific phosphotransferase enzyme IIA component), a phosphotransferase protein.

The term “CpdA” as used herein refers to a 3′,5′-cyclic adenosine monophosphate phosphodiesterase protein (also referred to as cyclic AMP phosphodiesterase, cAMP phosphodiesterase or icc) which hydrolyzes cAMP.

The term “CadD” as used herein refers to a 3′,5′-cyclic adenosine monophosphate deaminase protein (also referred to as ADD, cyclic adenylate deaminase, cyclic AMP deaminase or cAMP deaminase) which deaminates cAMP.

The term “FeS-cluster” as used herein refers to [2Fe-2S] or [4Fe-4S] clusters of the formulas:

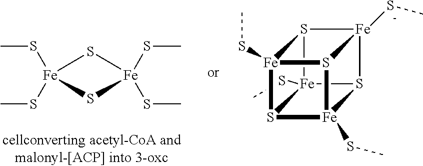

The term “FabG” as used herein refers to a 3-oxoacyl-[acyl-carrier-protein] reductase (EC: 1.1.1.100) converting a (3R)-hydroxyacyl-[ACP] into 3-oxoacyl-[ACP].

The term “FabZ” as used herein refers to a 3-hydroxyacyl-[acyl-carrier-protein] dehydratase (EC: 4.2.1.59) converting (3R)-hydroxyacyl-[ACP] into (2E)-enoyl-[ACP].

The term “Fabl” as used herein refers to an enoyl-[acyl-carrier-protein] reductase (EC: 1.3.1.9) converting 2,3-saturated acyl-[ACP] into (2E)-enoyl-[ACP].

The term “FabB” as used herein refers to a 3-oxoacyl-[acyl-carrier-protein] synthase (EC: 2.3.1.41) converting fatty acyl-[ACP] and malonyl-[ACP] into 3-oxoacyl-[ACP] and holo-[ACP].

The term “FabF” as used herein refers to a 3-oxoacyl-[acyl-carrier-protein] synthase (EC: 2.3.1.179) converting (11Z)-hexadecenoyl-[ACP] and malonyl-[ACP] into 3-oxo-(13Z)-octadecenoyl-[ACP] and holo-[ACP].

The term “BioC” as used herein refers to a malonyl-acyl carrier protein methyltransferase (EC2.1.1.197) converting Malonyl-acyl carrier protein into malonyl-acyl carrier protein methyl ester.

The term “BioF” as used herein refers to an 8-amino-7-oxononanoate synthase (EC2.3.1.47) converting a pimeloyl-acyl carrier protein into KAPA.

The term “BioA” as used herein refers to a Adenosylmethionine-8-amino-7-oxononanoate transaminase (EC2.6.1.62) converting KAPA into DABA using SAM as amino donor.

The term “Biok” as used herein refers to an adenosylmethionine-8-amino-7-oxononanoate transaminase converting KAPA into DAPA using lysine as amino donor.

The term “BioD” as used herein refers to a desthiobiotin synthase (EC6.3.3.3) converting DABA into DTB.

The term “Biol” as used herein refers to a biotin biosynthesis cytochrome P450, (pimeloyl-[acp] synthase (EC1.14.14.46) converting long-chain acyl-[acyl-carrier] protein into pimeloyl-[acp].

The term “BioW” as used herein refers a 6-carboxyhexanoate-CoA ligase (EC6.2.1.14) converting pimelate into pimeloyl-CoA.

The term “KAPA” as used herein refers to 7-keto-8-aminopelargonic acid.

The term “DAPA” as used herein refers to 7,8-Diaminopelargonic Acid.

The term “DTB” as used herein refers to desthiobiotin.

The term “SAM” as used herein refers to S-adenosyl-L-methionine.

The term “SAH” as used herein refers to S-Adenosyl-L-Homocysteine

The term “CoA” as used herein refers to coenzyme A

The term “ACP” as used herein refers to Acyl Carrier Protein

The term “AMTOB” as used herein refers to S-adenosyl-2-oxo-4-thiomethylbutyrate.

The term “5′DOA” as used herein refers to 5′-deoxyadenosine.

The term “TMP-phosphatase” as used herein refers to a thiamine monophosphate phosphatase dephosphorylating thiamine monophosphate to thiamine. It has been shown that for example the bifunctional TH2 protein from Arabidopsis thaliana has this activity (see also WO2017103221).

The term “ThiK” as used herein refers to a thiamine kinase that catalyzes the phosphorylation of thiamine to thiamine-monophosphate (TMP).

The term “ThiL” as used herein refers to a thiamine-monophosphate kinase. It catalyzes the ATP-dependent phosphorylation of thiamine-monophosphate (TMP) to form thiamine-pyrophosphate (TPP), the active form of vitamin B1. It cannot use thiamine as substrate. Is highly specific for ATP as phosphate donor.

The term “ThiM” as used herein refers to a Hydroxyethylthiazole kinase that catalyzes the phosphorylation of the hydroxyl group of 4-methyl-5-beta-hydroxyethylthiazole.

The term “ThiD” as used herein refers to a Hydroxymethylpyrimidine or phosphomethylpyrimidine kinase that catalyzes the phosphorylation of hydroxymethylpyrimidine phosphate (HMP-P) to HMP-PP, and of HMP to HMP-P. ThiD shows no activity with pyridoxal, pyridoxamine or pyridoxine.

The term “ThiC” as used herein refers to a Phosphomethylpyrimidine synthase protein that catalyzes the synthesis of the hydroxymethylpyrimidine phosphate (HMP-P) moiety of thiamine from aminoimidazole ribotide (AIR) in a radical S-adenosyl-L-methionine (SAM)-dependent reaction.

The term “ThiE” as used herein refers to a thiamine-phosphate synthase protein that condenses 4-methyl-5-(beta-hydroxyethyl)-thiazole monophosphate (THZ-P) and 2-methyl-4-amino-5-hydroxymethyl pyrimidine pyrophosphate (HMP-PP) to form thiamine monophosphate (TMP).

The term “ThiF” as used herein refers to a sulfur carrier protein ThiS adenylyltransferase. ThiF catalyzes the adenylation of the carboxy terminus of ThiS and the subsequent displacement of AMP catalyzed by Thil-persulfide to give a ThiS-Thil acyl disulfide ThiS.

The term “ThiS” as used herein refers to thiamine diphosphate biosynthesis gene that is involved in the pathway thiamine diphosphate biosynthesis, which is part of Cofactor biosynthesis.

The term “ThiG” as used herein refers to a Thiazole synthase protein that catalyzes the rearrangement of 1-deoxy-D-xylulose 5-phosphate (DXP) to produce the thiazole phosphate moiety of thiamine. Sulfur is provided by the thiocarboxylate moiety of the carrier protein ThiS.

The term “ThiH” as used herein refers to a 2-iminoacetate synthase protein that catalyzes the radical SAM-mediated cleavage of tyrosine to 2-iminoacetate and 4-cresol.

The term “Thil” as used herein refers to a tRNA sulfurtransferase protein that catalyzes the ATP-dependent transfer of a sulfur to tRNA to produce 4-thiouridine in position 8 of tRNAs, which functions as a near-UV photosensor. Also catalyzes the transfer of sulfur to the sulfur carrier protein ThiS, forming ThiS-thiocarboxylate. This is a step in the synthesis of thiazole, in the thiamine biosynthesis pathway. The sulfur is donated as persulfide by IscS.

The term “Dxs protein” as used herein refers to a 1-deoxy-D-xylulose-5-phosphate synthase that catalyzes the acyloin condensation reaction between C atoms 2 and 3 of pyruvate and glyceraldehyde 3-phosphate to yield 1-deoxy-D-xylulose-5-phosphate (DXP).

The term “ThiO” as used herein refers to a glycine oxidase that catalyzes the FAD-dependent oxidative deamination of various amines and D-amino acids to yield the corresponding alpha-keto acids, ammonia/amine, and hydrogen peroxide. It is essential for thiamine biosynthesis in organisms that generally lack ThiH since the oxidation of glycine catalyzed by ThiO also generates the glycine imine intermediate (dehydroglycine) required for the biosynthesis of the thiazole ring of thiamine pyrophosphate.

The term “IscR” as used herein refers to HTH-type transcriptional regulator IscR that regulates the transcription of several operons and genes involved in the biogenesis of Fe—S clusters and Fe—S-containing proteins. Transcriptional repressor of the iscRSUA operon, which is involved in the assembly of Fe—S clusters into Fe—S proteins.

The term “host cell” refers to any cell type that is susceptible to transformation, transfection, transduction, or the like with a nucleic acid construct or expression vector comprising a polynucleotide of the present invention. Host cell encompasses any progeny of a parent cell that is not identical to the parent cell due to mutations that occur during replication.

The term “polynucleotide construct” refers to a polynucleotide, either single- or double stranded, which is isolated from a naturally occurring gene or is modified to contain segments of nucleic acids in a manner that would not otherwise exist in nature or which is synthetic, and which comprises a polynucleotide encoding a polypeptide and one or more control sequences.

The term “operably linked” refers to a configuration in which a control sequence is placed at an appropriate position relative to the coding polynucleotide such that the control sequence directs expression of the coding polynucleotide.

The terms “nucleotide sequence” and “polynucleotide” are used herein interchangeably.

The term “coding sequence” refers to a nucleotide sequence, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

The term “control sequence” as used herein refers to a nucleotide sequence necessary for expression of a polynucleotide encoding a polypeptide. A control sequence may be native (i.e., from the same gene or organism) or heterologous or foreign (i.e., from a different or organism) to the polynucleotide encoding the polypeptide. Control sequences include, but are not limited to leader sequences, polyadenylation sequence, pro-peptide coding sequence, promoter sequences, signal peptide coding sequence, translation terminator (stop) sequences and transcription terminator (stop) sequences. To be operational control sequences usually must include promoter sequences, transcriptional and translational stop signals. Control sequences may be provided with linkers for the purpose of introducing specific restriction sites facilitating ligation of the control sequences with a coding region of a polynucleotide encoding a polypeptide.

The term “expression vector” refers to a DNA molecule, either single- or double stranded, either linear or circular, which comprises a polynucleotide encoding a polypeptide and is operably linked to control sequences that provide for its expression. Expression vectors include expression cassettes for the integration of genes into a host cell as well as plasmids and/or chromosomes comprising such genes.

The term “expression” includes any step involved in the production of a polypeptide including, but not limited to, transcription, post-transcriptional modification, translation, post-translational modification, and secretion.

The terms “heterologous” or “recombinant” or “genetically modified” and their grammatical equivalents as used herein interchangeably refers to entities “derived from a different species or cell”. For example, a heterologous or recombinant polynucleotide gene is a gene in a host cell not naturally containing that gene, i.e., the gene is from a different species or cell type than the host cell. The terms as used herein about microbial host cells refers to microbial host cells comprising and expressing heterologous or recombinant polynucleotide genes.

The term “metabolic pathway” as used herein is intended to mean two or more enzymes acting sequentially in a live cell to convert chemical substrate(s) into chemical product(s). Enzymes are characterized by having catalytic activity, which can change the chemical structure of the substrate(s).

An enzyme may have more than one substrate and produce more than one product. The enzyme may also depend on cofactors, which can be inorganic chemical compounds or organic compounds such as proteins for example enzymes (co-enzymes). A cytochrome P450 reductase (CPR) that reduces cofactors such as NADPH in certain cytochrome P450 enzymes is an example of an enzymatic co-factor. The term “operative biosynthetic metabolic pathway” refers to a metabolic pathway that occurs in a live recombinant host, as described herein.

The term “in vivo”, as used herein refers to within a living cell or organism, including, for example animal, a plant or a microorganism.

The term “in vitro”, as used herein refers to outside a living cell or organism, including, without limitation, for example, in a microwell plate, a tube, a flask, a beaker, a tank, a reactor and the like.

The term “substrate” or “precursor”, as used herein refers to any compound that can be converted into a different compound. For example, desthiobiotin can be a substrate for biotin synthase and can be converted into biotin. For clarity, substrates and/or precursors include both compounds generated in situ by an enzymatic reaction in a cell or exogenously provided compounds, such as exogenously provided organic molecules which the host cell can metabolize into a desired compound.

Term “endogenous” or “native” as used herein refers to a gene or a polypeptide in a host cell which originates from the same host cell.

The term “deletion” as used herein refers to manipulation of a gene so that it is no longer expressed in a host cell.

The term “disruption” as used herein refers to manipulation of a gene or any of the machinery participating in the expression the gene, so that it is no longer expressed in a host cell.

The term “attenuation” as used herein refers to manipulation of a gene or any of the machinery participating in the expression the gene, so that it the expression of the gene is reduced as compared to expression without the manipulation.

The terms “substantially” or “approximately” or “about”, as used herein refers to a reasonable deviation around a value or parameter such that the value or parameter is not significantly changed. These terms of deviation from a value should be construed as including a deviation of the value where the deviation would not negate the meaning of the value deviated from. For example, in relation to a reference numerical value the terms of degree can include a range of values plus or minus 10% from that value. For example, deviation from a value can include a specified value plus or minus a certain percentage from that value, such as plus or minus 9%, 8%, 7%, 6%, 5%, 4%, 3%, 2%, or 1% from the specified value.

The term “and/or” as used herein is intended to represent an inclusive “or”. The wording X and/or Y is meant to mean both X or Y and X and Y. Further the wording X, Y and/or Z is intended to mean X, Y and Z alone or any combination of X, Y, and Z.

The term “isolated” as used herein about a compound, refers to any compound, which by means of human intervention, has been put in a form or environment that differs from the form or environment in which it is found in nature. Isolated compounds include but is no limited to compounds of the invention for which the ratio of the compounds relative to other constituents with which they are associated in nature is increased or decreased. In an important embodiment the amount of compound is increased relative to other constituents with which the compound is associated in nature. In an embodiment the compound of the invention may be isolated into a pure or substantially pure form. In this context, a substantially pure compound means that the compound is separated from other extraneous or unwanted material present from the onset of producing the compound or generated in the manufacturing process. Such a substantially pure compound preparation contains less than 10%, such as less than 8%, such as less than 6%, such as less than 5%, such as less than 4%, such as less than 3%, such as less than 2%, such as less than 1%, such as less than 0.5% by weight of other extraneous or unwanted material usually associated with the compound when expressed natively or recombinantly. In an embodiment, the isolated compound is at least 90% pure, such as at least 91% pure, such as at least 92% pure, such as at least 93% pure, such as at least 94% pure, such as at least 95% pure, such as at least 96% pure, such as at least 97% pure, such as at least 98% pure, such as at least 99% pure, such as at least 99.5% pure, such as 100% pure by weight.

The term “% identity” is used herein about the relatedness between two amino acid sequences or between two nucleotide sequences. “% identity” as used herein about amino acid sequences refers to the degree of identity in percent between two amino acid sequences obtained when using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

$$\frac{identical amino acid residues}{Length of alignment - total number of gaps in alignment} \times 100$$

The term “% identity” as used herein about nucleotide sequences refers to the degree of identity in percent between two nucleotide sequences obtained when using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

$$\frac{identical deoxryibonucleotides}{Length of alignment - total number of gaps in alignment} \times 1 \; 00$$

The protein sequences of the present invention can further be used as a “query sequence” to perform a search against sequence databases, for example to identify other family members or related sequences. Such searches can be performed using the BLAST programs. Software for performing BLAST analyses is publicly available through the National Center for Biotechnology Information (http://www.ncbi.nlm.nih.gov). BLASTP is used for amino acid sequences and BLASTN for nucleotide sequences. The BLAST program uses as defaults:
Cost to open gap: default=5 for nucleotides/11 for proteins
Cost to extend gap: default=2 for nucleotides/1 for proteins
Penalty for nucleotide mismatch: default=−3
Reward for nucleotide match: default=1
Expect value: default=10
Wordsize: default=11 for nucleotides/28 for megablast/3 for proteins.

Furthermore, the degree of local identity between the amino acid sequence query or nucleic acid sequence query and the retrieved homologous sequences is determined by the BLAST program. However only those sequence segments are compared that give a match above a certain threshold. Accordingly, the program calculates the identity only for these matching segments. Therefore, the identity calculated in this way is referred to as local identity.

The term “mature polypeptide” or “mature enzyme” as used herein refers to a polypeptide in its final active form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide.

The term “cDNA” refers to a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

The terms “comprise” and “include” as used throughout the specification and the accompanying items as well as variations such as “comprises”, “comprising”, “includes” and “including” are to be interpreted inclusively. These words are intended to convey the possible inclusion of other elements or integers not specifically recited, where the context allows.

The articles “a” and “an” are used herein refers to one or to more than one (i.e., to one or at least one) of the grammatical object of the article. By way of example, “an element” may mean one element or more than one element.

Terms like “preferably”, “commonly”, “particularly”, and “typically” are not utilized herein to limit the scope of the itemed invention or to imply that certain features are critical, essential, or even important to the structure or function of the itemed invention. Rather, these terms are merely intended to highlight alternative or additional features that can or cannot be utilized in a particular embodiment of the present invention.

The term “cell culture” as used herein refers to a culture medium comprising a plurality of host cells of the invention. A cell culture may comprise a single strain of host cells or may comprise two or more distinct host cell strains. The culture medium may be any medium that may comprise a recombinant host, e.g., a liquid medium (i.e., a culture broth) or a semi-solid medium, and may comprise additional components, e.g., one or more of (i) trace metals; (ii) vitamins; (iii) salts (such as salts of phosphate, magnesium, potassium, zinc, iron); (iv) nitrogen sources (such as YNB, ammonium sulfate, urea, yeast extracts, ammonium nitrate, ammonium chloride, malt extract, peptone and/or amino acids); (v) carbon source (such as dextrose, sucrose, glycerol, glucose, maltose, molasses, starch, cellulose, xylan, pectin, lignocellolytic biomass hydrolysate, and/or acetate); (vi) nucleobases; (vii) aminoglycosides; and/or (viii) antibiotics (such as G418 and hygromycin B).

The term “radical SAM” as used herein refers to a superfamily of enzymes that use a [4Fe-4S]⁺ cluster to reductively cleave S-adenosyl-L-methionine (SAM) to generate a radical, usually a 5′-deoxyadenosyl radical, as a critical intermediate. The vast majority of known radical SAM enzymes have a cysteine-rich motif that matches or resembles CxxxCxxC.

### Genetically Modified Host Cells

The genetical modification of the host cells provided for in the first aspect having increased production of vitamin B compounds, include in some embodiments one or more mutations in native polynucleotide constructs encoding one or more proteins selected from protein cAMP receptor protein (CRP), carbohydrate repression resistance protein (CRR) and adenylate cyclase protein (CyaA).

The increase in the genetically modified host cells capacity to produce vitamin B compounds can in some embodiments be at least 50%, such as at least 100%, such as least 150%, such as at least 200%.

Mutations in the native polynucleotide constructs preferably include deletions, disruption, and/or an attenuation of the gene and particularly the deletions, disruptions and/or attenuations comprise a full or partial deletion of the gene, a translational knockout through introduction of a stop codon or a frameshift mutation. In one embodiment, the mutation is a deletion through complete removal of the gene or a translational knockout by introducing one or more stop codons or frameshift mutations preventing expression of an active peptide. In another embodiment the deletion, disruption and/or attenuation may be a point mutation in the polynucleotide constructs made in a promoter for the protein encoding gene, in the RBS region and/or in protein encoding sequence. In one particular embodiment, such point mutation is made in the sequence encoding the active site of the CyaA enzyme and reduces the activity of CyaA while in another embodiment such point mutation is made in the sequence encoding the cAMP binding moieties of CRP to reduce the affinity of CRP for cAMP. In another embodiment the phosphorylation site of CRR is mutated, preventing the formation of phosphorylated EIIA^{GIc}, and thereby activation of CyaA.

The CRP, native or mutated, is preferably at least 70%, such as at least 80%, such as at least 90%, such as at least 95%, such as at least 96%, such as at least 97%, such as at least 98%, such as at least 99%, such as 100% identical to the CRP comprised in SEQ ID NO: 39 or 97. The CRR, native or mutated is preferably at least 70%, such as at least 80%, such as at least 90%, such as at least 95%, such as at least 96%, such as at least 97%, such as at least 98%, such as at least 99%, such as 100% identical to the CRR comprised in SEQ ID NO: 41 or 99. The CyaA, native or mutated is preferably at least 70%, such as at least 80%, such as at least 90%, such as at least 95%, such as at least 96%, such as at least 97%, such as at least 98%, such as at least 99%, such as 100% identical to the CyaA comprised in SEQ ID NO: 43 or 101.

In some embodiments the gene encoding the CRP, native or mutated, is preferably least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 40 or 98 or genomic DNA thereof encoding the CRP comprised in SEQ ID NO: 39 or 97. The CRR, native or mutated, is preferably least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 42 or 100 or genomic DNA thereof encoding the CRR comprised in SEQ ID NO: 41 or 99. The CyaA, native or mutated, is preferably least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 44 or 102 or genomic DNA thereof encoding the CyaA comprised in SEQ ID NO: 43 or 101.

In some embodiments the encoded mutant CRP comprises a mutation in one or more positions corresponding to T12, D138, T146, F69, R82, V139, G57, K58, E59, M60, 161, L62, S63, G72, E73, L74, R83, S84, T128, S129, A136, F137, Q171, E172, 1173, G174, Q175, 1176, V177, G178, C179, S180, R181, E182, T183, V184, G185, R186 of SEQ ID NO: 39 or 97.

In some embodiments the encoded mutant CRR comprises a mutation in one or more positions corresponding to H76, H91 of SEQ ID NO: 41 or 99;

In some embodiments the encoded mutant CyaA comprise a mutation in the position corresponding to G60, K59, L63, T65, R188, G195, K196, R192, S103, S113, D114, D116, W118, E185, T189, K260, K264, K332, W200, D300 of SEQ ID NO: 43 or 101.

In the alternative the one or more genetic alterations increasing the non-CRP polypeptide binding of cAMP and/or degradation of cAMP in the host cell, may comprise:
a) introducing one or more heterologous polypeptides, which are not CRP, binding cAMP or cAMP degrading enzymes into the host cell;
b) overexpressing one or more native polypeptides binding cAMP which are not CRP or cAMP degrading enzymes in the host cell; and/or
c) mutating one or more native non-CRP cAMP binding polypeptides or cAMP degrading enzymes in the host cell to increase their cAMP binding and/or degradation capability.

In particular the one or more non-CRP cAMP binding proteins and/or cAMP degrading enzymes of a) can be selected among heterologous cAMP phosphodiesterase (CpdA) and cAMP deaminase (CadD). Where the host cell is E. coli or many other host cells, the CpdA and/or CadD must be introduced heterologously to the host cell using methods known in the art. In some embodiments wherein the over-expression of b) can comprise a cis-modification in the genome or a trans-modification in a plasmid, while in other embodiments the mutation of c) is a point mutation in a promoter for the protein encoding sequence, in the RBS region and/or in protein encoding sequence. Such point mutations can be made in the sequence encoding the active site of the for example CpdA and or CadD increasing the activity of CpdA and/or CadD. The CpdA may be at least 70%, such as at least 80%, such as at least 90%, such as at least 95%, such as at least 96%, such as at least 97%, such as at least 98%, such as at least 99%, such as 100% identical to the CpdA comprised in SEQ ID NO: 45 or 103; while the CadD may be at least 70%, such as at least 80%, such as at least 90%, such as at least 95%, such as at least 96%, such as at least 97%, such as at least 98%, such as at least 99%, such as 100% identical to the CadD comprised in SEQ ID NO: 47 or 105. More particularly the gene encoding the CpdA may be least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 46 or 104 or genomic DNA thereof encoding the CpdA comprised in SEQ ID NO: 45 or 105; while the gene encoding the CadD may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 48 or 106 or genomic DNA thereof encoding the CadD comprised in SEQ ID NO: 47 or 105.

The host cell of the invention further comprises an operative metabolic pathway comprising one or more native or heterologous pathway elements producing the vitamin B compound, and in particular such pathway elements comprise one or more one or more FeS cluster dependent enzymes, in particular radical SAM enzymes.

### Biotin Branch

In one preferred embodiment the vitamin B compound is biotin and the host cell comprise an operative metabolic pathway comprising one or more native or heterologous pathway elements producing the biotin. In this context the term “pathway elements” include any step or element relevant for the functioning of the pathway, including proteins, polypeptides, peptides, enzymes, co-factors or the like. The pathway elements for producing biotin can include one or more pathway elements are selected from:
a) one or more fatty acid synthesis enzymes selected from FabH, FabG, FabA, FabZ, Fabl, FabB and FabF;
b) a malonyl-acyl carrier protein methyltransferase (BioC) converting Malonyl-acyl carrier protein to malonyl-acyl carrier protein methyl ester;
c) a pimelyl-acyl carrier protin methyl ester esterase (BioH) converting O-methylpimeloyl-acyl carrier protein to pimeloyl-acyl carrier protein;
d) a 8-amino-7-oxononanoate synthase (BioF) converting Pimeloyl-acyl carrier protein to KAPA;
e) an adenosylmethionine-8-amino-7-oxononanoate transaminase (BioA) converting KAPA to DAPA using SAM as amino donor;
f) an adenosylmethionine-8-amino-7-oxononanoate transaminase (BioK) capable of converting KAPA into DAPA using lysine as amino donor;
g) a desthiobiotin synthase (BioD) converting DAPA to DTB;
h) a biotin biosynthesis cytochrome P450, (pimeloyl-[acp] synthase (Biol) converting long-chain acyl-[acyl-carrier protein to pimeloyl-[acp];
i) a 6-carboxyhexanoate-CoA ligase (BioW) converting pimelate+CoA to pimeloyl−CoA;
j) an FeS cluster Transcription factor polypeptide (IscR) capable of regulating an operon [isc operon] producing a FeS cluster co-factor; and
k) a biotin synthase (BioB) converting DTB to Biotin.

In some embodiments of the biotin pathway
the BioC may have at least 70%, such at least 75%, such as at least 80%, such as at least 90%, such as at least 95%, such as at least 99%, such as 100% identity to the BioC comprised in SEQ ID NO: 1;
the BioH may have at least 70%, such at least 75%, such as at least 80%, such as at least 90%, such as at least 95%, such as at least 99%, such as 100% identity to the BioH comprised in SEQ ID NO: 3;
the BioF may have has at least 70%, such at least 75%, such as at least 80%, such as at least 90%, such as at least 95%, such as at least 99%, such as 100% identity to the BioF comprised in SEQ ID NO: 5;
the BioA may have at least 70%, such at least 75%, such as at least 80%, such as at least 90%, such as at least 95%, such as at least 99%, such as 100% identity to the BioA comprised in SEQ ID NO: 7;
the Biok may have at least 70%, such at least 75%, such as at least 80%, such as at least 90%, such as at least 95%, such as at least 99%, such as 100% identity to the Biok comprised in SEQ ID NO: 9;
the BioD may have at least 70%, such at least 75%, such as at least 80%, such as at least 90%, such as at least 95%, such as at least 99%, such as 100% identity to the BioD comprised in SEQ ID NO: 11;
the Biol may have at least 70%, such at least 75%, such as at least 80%, such as at least 90%, such as at least 95%, such as at least 99%, such as 100% identity to the Biol comprised in SEQ ID NO: 13;
the BioW may have at least 70%, such at least 75%, such as at least 80%, such as at least 90%, such as at least 95%, such as at least 99%, such as 100% identity to the BioW comprised in SEQ ID NO: 15;
the IscR may have at least 70%, such at least 75%, such as at least 80%, such as at least 90%, such as at least 95%, such as at least 99%, such as 100% identity to the IscR comprised in SEQ ID NO: 17, 19, 21, 23, 25, 27, 29, 31, 33, or 35; and/or
the BioB may have at least 70%, such at least 75%, such as at least 80%, such as at least 90%, such as at least 95%, such as at least 99%, such as 100% identity to the T1BioB comprised in SEQ ID NO: 37.

The gene encoding the BioC of the biotin pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 2 or genomic DNA thereof encoding the BioC comprised in SEQ ID NO: 1; the gene encoding the BioH of the biotin pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 4 or genomic DNA thereof encoding the BioH comprised in SEQ ID NO: 3; the gene encoding the BioF of the biotin pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 6 or genomic DNA thereof encoding the BioF comprised in SEQ ID NO: 5; the gene encoding the BioA of the biotin pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 8 or genomic DNA thereof encoding the BioA comprised in SEQ ID NO: 7; the gene encoding the Biok of the biotin pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 10 or genomic DNA thereof encoding the Biok comprised in SEQ ID NO: 9; the gene encoding the BioD of the biotin pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 12 or genomic DNA thereof encoding the BioD comprised in SEQ ID NO: 11; the gene encoding the Biol of the biotin pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 14 or genomic DNA thereof encoding the Biol comprised in SEQ ID NO: 13; the gene encoding the BioW of the biotin pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 16 or genomic DNA thereof encoding the BioW comprised in SEQ ID NO: 15; the gene encoding the IscR of the biotin pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 18, 20, 22, 24, 26, 28, 30, 32, 34 or 36 or genomic DNA thereof encoding the IscR comprised in SEQ ID NO: 17, 19, 21, 23, 25, 27, 29, 31, 33, or 35 respectively; and the gene encoding the BioB of the biotin pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 38 or genomic DNA thereof encoding the BioB comprised in SEQ ID NO: 37.

### Thiamine Branch

In another preferred embodiment the vitamin B compound is thiamine and the host cell further comprises an operative metabolic pathway comprising one or more native or heterologous pathway elements producing the thiamine, and in particular such pathway elements comprise one or more thiamine mono-phosphate phosphatase enzymes. In this context the term “pathway elements” include any element relevant for the functioning of the pathway, including proteins, polypeptides, peptides, enzymes, co-factors or the like. In some embodiments the pathway elements for producing thiamine can include one or more pathway elements are selected from:
a) one or more phosphate synthase enzymes selected from phosphomethylpyrimidine synthase (ThiC); that catalyzes the synthesis of the hydroxymethylpyrimidine phosphate (HMP-P) moiety of thiamine from aminoimidazole ribotide (AIR) in a radical S-adenosyl-L-methionine (SAM)-dependent reaction;
b) a hydroxymethylpyrimidine/phosphomethylpyrimidine kinase (ThiD) that catalyzes the phosphorylation of hydroxymethylpyrimidine phosphate (HMP-P) to HMP-PP, and of HMP to HMP-P;
c) a sulfur carrier protein (ThiF) that catalyzes the adenylation of the carboxy terminus of ThiS and the subsequent displacement of AMP catalyzed by Thil-persulfide to give a ThiS-Thil acyl disulfide ThiS;
d) a thiamine diphosphate (ThiS) that catalyzes the adenylation of the carboxy terminus of ThiS and the subsequent displacement of AMP catalyzed by Thil-persulfide to give a ThiS-Thil acyl disulfide ThiS;
e) a 2-iminoacetate synthase (ThiH) converting that catalyzes the radical-mediated cleavage of tyrosine to 2-iminoacetate and 4-cresol;
f) a thiazole synthase (ThiG) that catalyzes the rearrangement of 1-deoxy-D-xylulose 5-phosphate (DXP) to produce the thiazole phosphate moiety of thiamine;
g) a hydroxyethylthiazole kinase (ThiM) that catalyzes the rearrangement of 1-deoxy-D-xylulose 5-phosphate (DXP) to produce the thiazole phosphate moiety of thiamine;
h) a thiamine mono-phosphate (TMP) phosphatase that dephosphorylate thiamine monophosphate to thiamine;
i) a thiamine kinase (ThiK) that catalyzes the phosphorylation of thiamine to thiamine phosphate;
j) a thiamine-monophosphate kinase (ThiL) which catalyzes the ATP-dependent phosphorylation of thiamine-monophosphate (TMP) to form thiamine-pyrophosphate (TPP);
k) a thiamine-phosphate synthase (ThiE) that condenses 4-methyl-5-(beta-hydroxyethyl) thiazole monophosphate (THZ-P) and 2-methyl-4-amino-5-hydroxymethyl pyrimidine pyrophosphate (HMP-PP) to form thiamine monophosphate (TMP);
l) a tRNA sulfurtransferase (Thil) that catalyzes the ATP-dependent transfer of a sulfur to tRNA to produce 4-thiouridine in position 8 of tRNAs;
m) an FeS cluster Transcription factor polypeptide (IscR) capable of regulating an operon [isc operon] producing a FeS cluster co-factor;
n) a glycine oxidase (ThiO) that catalyzes the FAD-dependent oxidative deamination of various amines and D-amino acids to yield the corresponding alpha-keto acids, ammonia/amine; hydrogen peroxide.

In some embodiments of the thiamine pathway
the ThiC may have at least 70%, such at least 75%, such as at least 80%, such as at least 90%, such as at least 95%, such as at least 99%, such as 100% identity to the ThiC comprised in SEQ ID NO: 51;
the ThiD may have at least 70%, such at least 75%, such as at least 80%, such as at least 90%, such as at least 95%, such as at least 99%, such as 100% identity to the ThiD comprised in SEQ ID NO: 53;
the ThiF may have at least 70%, such at least 75%, such as at least 80%, such as at least 90%, such as at least 95%, such as at least 99%, such as 100% identity to the ThiF comprised in SEQ ID NO: 55;
the ThiS may have at least 70%, such at least 75%, such as at least 80%, such as at least 90%, such as at least 95%, such as at least 99%, such as 100% identity to the ThiS comprised in SEQ ID NO: 57;
the ThiH may have at least 70%, such at least 75%, such as at least 80%, such as at least 90%, such as at least 95%, such as at least 99%, such as 100% identity to the ThiH comprised in SEQ ID NO: 59;
the ThiG may have at least 70%, such at least 75%, such as at least 80%, such as at least 90%, such as at least 95%, such as at least 99%, such as 100% identity to the ThiG comprised in SEQ ID NO: 61;
the ThiM may have at least 70%, such at least 75%, such as at least 80%, such as at least 90%, such as at least 95%, such as at least 99%, such as 100% identity to the ThiM comprised in SEQ ID NO: 63;
the TMP phosphatase may have at least 70%, such at least 75%, such as at least 80%, such as at least 90%, such as at least 95%, such as at least 99%, such as 100% identity to the TMP phosphatase comprised in SEQ ID NO: 65;
the Thik may have at least 70%, such at least 75%, such as at least 80%, such as at least 90%, such as at least 95%, such as at least 99%, such as 100% identity to the Thik comprised in SEQ ID NO: 67;
the ThiL may have at least 70%, such at least 75%, such as at least 80%, such as at least 90%, such as at least 95%, such as at least 99%, such as 100% identity to the ThiL comprised in SEQ ID NO: 69;
the ThiE may have at least 70%, such at least 75%, such as at least 80%, such as at least 90%, such as at least 95%, such as at least 99%, such as 100% identity to the ThiE comprised in SEQ ID NO: 71;
the Thil may have at least 70%, such at least 75%, such as at least 80%, such as at least 90%, such as at least 95%, such as at least 99%, such as 100% identity to the Thil comprised in SEQ ID NO: 73;
the IscR may have at least 70%, such at least 75%, such as at least 80%, such as at least 90%, such as at least 95%, such as at least 99%, such as 100% identity to the IscR comprised in SEQ ID NO: 77, 79, 81, 83, 85, 87, 89, 91, 93 or 95; and/or
the ThiO may have at least 70%, such at least 75%, such as at least 80%, such as at least 90%, such as at least 95%, such as at least 99%, such as 100% identity to ThiO comprised in SEQ ID NO: 109, 111, 113 or 115.

### The Gene Encoding

the ThiC of the thiamine pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 52 or genomic DNA thereof encoding the ThiC comprised in SEQ ID NO: 51;
the gene encoding the ThD of the thiamine pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 54 or genomic DNA thereof encoding the ThiD comprised in SEQ ID NO: 53;
the gene encoding the ThiF of the thiamine pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 56 or genomic DNA thereof encoding the ThiF comprised in SEQ ID NO: 55;
the gene encoding the ThiS of the thiamine pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 58 or genomic DNA thereof encoding the ThiS comprised in SEQ ID NO: 57;
the gene encoding the ThiH of the thiamine pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 60 or genomic DNA thereof encoding the ThiH comprised in SEQ ID NO: 59;
the gene encoding the ThiG of the thiamine pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 62 or genomic DNA thereof encoding the ThiG comprised in SEQ ID NO: 61;
the gene encoding the ThiM of the thiamine pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 64 or genomic DNA thereof encoding the ThiM comprised in SEQ ID NO: 63;
the gene encoding the TMP phosphatase of the thiamine pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 66 or genomic DNA thereof encoding the TMP phosphatase comprised in SEQ ID NO: 65;
the gene encoding the ThiK of the thiamine pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 68 or genomic DNA thereof encoding the Thik comprised in SEQ ID NO: 67;
the gene encoding the ThiL of the thiamine pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 70 or genomic DNA thereof encoding the ThiL comprised in SEQ ID NO: 69;
the gene encoding the ThiE of the thiamine pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 72 or genomic DNA thereof encoding the ThiE comprised in SEQ ID NO: 71; and
the gene encoding the Thil of the thiamine pathway may be at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 74 or genomic DNA thereof encoding the Thil comprised in SEQ ID NO: 73;
the gene encoding the IscR of the thiamine pathway may be a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 76, 78, 80, 82, 84, 86, 88, 90, 92, 94 or 96, or genomic DNA thereof encoding the IscR comprised in SEQ ID NO: 75, 77, 79, 81, 83, 85, 87, 89, 91, 93 or 95; and/or
the gene encoding the ThiO of the thiamine pathway may be a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 110, 112, 114 or 116, or genomic DNA thereof encoding the ThiO comprised in SEQ ID NO: 109, 111, 113 or 115.

### B₃ Branch

In other embodiments the vitamin B compound is one or more vitamins in the B₃ complex and the host cell disclosed herein further comprises an operative metabolic pathway comprising one or more native or heterologous pathway elements producing ome or more B₃ vitamins. In this context the term “pathway elements” include any step or element relevant for the functioning of the pathway, including proteins, polypeptides, peptides, enzymes, co-factors or the like. B₃ complex includes B3 vitamins nicotinamide riboside (NR), nicotinic acid (NA), nicotinamide (NA), nicotinamide mononucleotide (NMN), nicotinamide adenine dinucleotide (NAD), or the intermediate quinolate.

In some embodiments the pathway elements for producing B₃ vitamins can include one or more pathway elements are selected from:
a) NadA quinolate synthase (EC: 2.5.1.72);
b) NadE nicotinic acid mononucleotide amidase;
c) NMN nucleosidase (EC: 3.2.2.14);
d) pncA deamidase (EC: 3.5.1.19);
e) NadB aspartate oxidase (EC: 1.4.3.16);
f) NadC nicotinate-nucleotide pyrophosphorylase (EC: 2.4.2.19);
g) AphA Class B acid phosphatase; and/or
h) an FeS cluster Transcription factor polypeptide (IscR) capable of regulating an operon [isc operon] producing a FeS cluster co-factor.

In some embodiments of the B₃ pathway
the NadA quinolate synthase may have at least 70%, such at least 75%, such as at least 80%, such as at least 90%, such as at least 95%, such as at least 99%, such as 100% identity to the NadA quinolate synthase comprised in SEQ ID NO: 140, 141, 142, 143, 144, 145, 146, 147, 148, 149 or 150;
the NadE nicotinic acid mononucleotide amidase may have at least 70%, such at least 75%, such as at least 80%, such as at least 90%, such as at least 95%, such as at least 99%, such as 100% identity to the NadE nicotinic acid mononucleotide amidase comprised in SEQ ID NO: 152;
the NMN nucleosidase may have at least 70%, such at least 75%, such as at least 80%, such as at least 90%, such as at least 95%, such as at least 99%, such as 100% identity to the NMN nucleosidase comprised in SEQ ID NO: 155;
the pncA deamidase may have at least 70%, such at least 75%, such as at least 80%, such as at least 90%, such as at least 95%, such as at least 99%, such as 100% identity to the pncA deamidase comprised in SEQ ID NO: 154;
the NadB aspartate oxidase may have at least 70%, such at least 75%, such as at least 80%, such as at least 90%, such as at least 95%, such as at least 99%, such as 100% identity to the NadB aspartate oxidase comprised in SEQ ID NO: 151;
the NadC nicotinate-nucleotide pyrophosphorylase may have at least 70%, such at least 75%, such as at least 80%, such as at least 90%, such as at least 95%, such as at least 99%, such as 100% identity to the NadC nicotinate-nucleotide pyrophosphorylase comprised in SEQ ID NO: 157;
the AphA Class B acid phosphatase may have at least 70%, such at least 75%, such as at least 80%, such as at least 90%, such as at least 95%, such as at least 99%, such as 100% identity to the AphA Class B acid phosphatase comprised in SEQ ID NO: 153, and/or
the IscR may have at least 70%, such at least 75%, such as at least 80%, such as at least 90%, such as at least 95%, such as at least 99%, such as 100% identity to the IscR comprised in SEQ ID NO: 77, 79, 81, 83, 85, 87, 89, 91, 93 or 95.

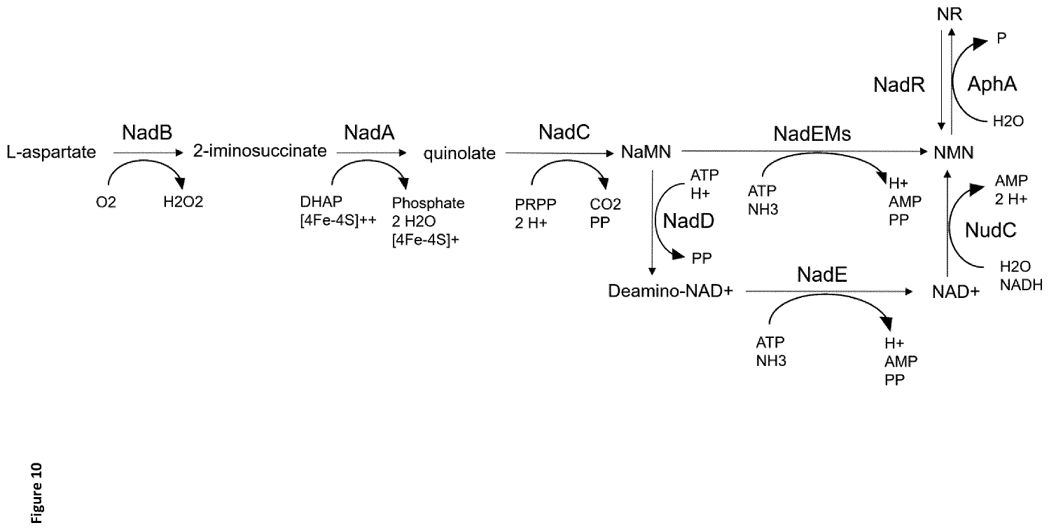

The NadA may be transgene or an up-regulated nadA endogenous gene. The NadE nicotinic acid mononucleotide amidase can synthesize both quinolate and NR (FIG. 10). Additionally, the inclusion of one or more of the NMN nucleosidase and/or pncA nicotinamide deamidase activity will allow the cells to synthesize both NA and NAM (FIG. 10). Optionally, the cells may comprise one of more transgenes or upregulated endogenous genes (as defined herein) encoding polypeptides that catalyze other steps in the NR synthesis pathway (FIG. 10), such as a NadB polypeptide having aspartate oxidase activity (EC: 1.4.3.16). Preferably, the NAD salvage pathway is down-regulated, for example by deletion or inactivation of the nadR and/or pncC genes in the genetically modified prokaryotic cell, thereby reducing NR consumption.

An increase in the levels of those polypeptides that catalyze steps in the B₃ (NR) pathway enhances the synthesis of both intermediates and end products of the NR pathway in the host cell. When the gene(s) encoding quinolate synthase together with one or more additional polypeptides that catalyze additional steps in the quinolate and B₃ vitamins pathways are transgenes, they are preferably located in the genome of the genetically modified prokaryotic cell, either integrated into the host cell chromosome or on a self-replicating plasmid. The transgene encoding NadA and one or more of the transgenes (nadB, and nadE) encoding enzymes in the NR pathway enzymes may be present in the genome within one or more operons.

The promoter driving expression of the transgene encoding NadB and one or more additional transgenes is preferably a non-native promoter, which may be a heterologous constitutive-promoter or an inducible-promoter. When expression driven by the promoter is constitutive, then a suitable promoter includes apFab family SEQ ID NO: 138 while a suitable inducible promoter includes the lac promoter lac p, which is regulated by repressor lacl SEQ ID NO: 135. Suitable terminators include members of the apFAB terminator family including SEQ ID NO: 139. The selected promoter and terminator may be operably linked to the respective gene, either to provide individual gene regulation or for regulation of an operon.

B₃ vitamins and quinolate can be produced using genetically modified host cells such as described in example 4 of WO2020148351 further modified as described herein.

### B12 Branch

In other embodiments the vitamin B compound is vitamin B₁₂ cobalamin, and the host cell disclosed herein further comprises an operative metabolic pathway comprising one or more native or heterologous pathway elements producing vitamin B₁₂. In this context the term “pathway elements” include any step or element relevant for the functioning of the pathway, including proteins, polypeptides, peptides, enzymes, co-factors or the like. Host cells described herein genetically modified to lack or down-regulate CRP-cAMP complex and modified with a transgene or up-regulated native cobG gene and additional transgenes or up-regulated native genes have an enhanced flux through the B₁₂ pathway, and enhanced production of the intermediate hydrogenobyrinic acid (HBA).

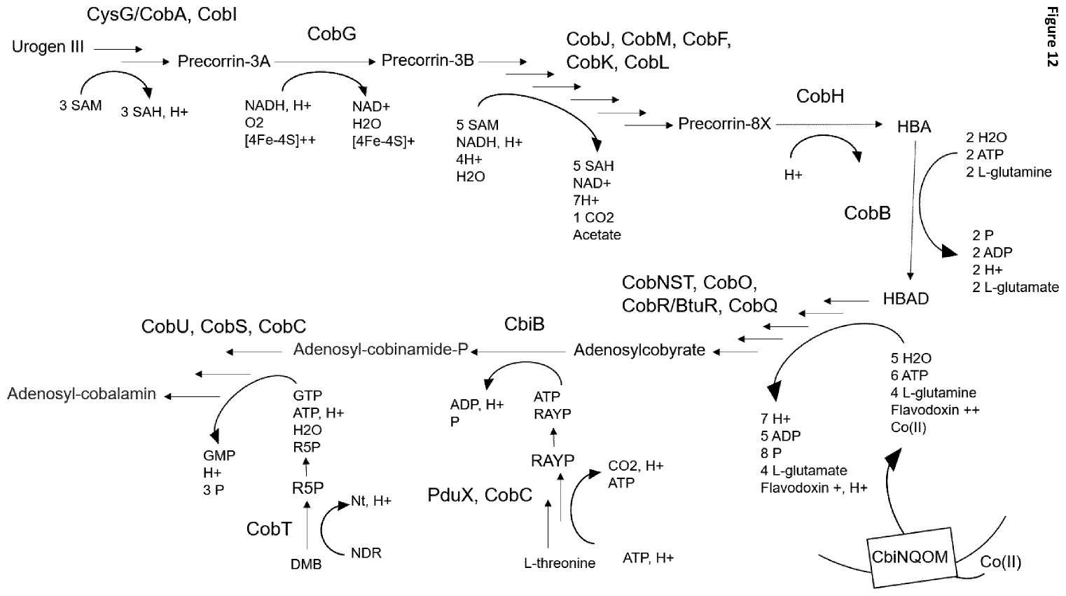

Host cells further modified to comprise transgenes encoding polypeptides that catalyze the subsequent steps in the cob synthesis pathway (see FIG. 12), in particular genes encoding CobNST, CobC, CobD, CobT, Pdux, CobU, CobS, CbiB, CbiN, CbiQ, CbiO, and CbiM have enhanced production of cobalamin.

Accordingly, in some embodiments the pathway elements for producing vitamin B₁₂ can include one or more pathway elements are selected from:
a) CobG precorrin-3B synthase (EC: 1.14.13.83);
b) Cobl precorrin-2 C20-methyltransferase (EC: 2.1.1.130), converting precorrin-2 into precorrin-3A;
c) CobM precorrin-3 methylase (EC: 2.1.1.133) catalyzing the synthesis of precorrin-5 from precorrin-4;
d) CobF cobalt-precorrin-6A synthase (EC: 2.1.1.195) catalyzing the synthesis of precorrin-6A from precorrin-5B;
e) CobK precorrin-6A reductase (EC: 1.3.1.54) catalyzing the synthesis of precorrin-6B from precorrin-6A;
f) CobH precorrin isomerase (EC: 5.4.99.61) catalyzing the conversion of precorrin-8X to hydrogenobyrinate;
g) CobL Precorrin-6Y C (5,15)-methyltransferase (EC: 2.1.1.132) catalyzing the conversion of C-5 and C-15 in precorrin-6Y to form precorrin-8X;
h) CobJ Precorrin-3B C (17)-methyltransferase (EC: 2.1.1.131) catalyzing the methylation of precorrin-3B to form precorrin-4;
i) CobN subunit of aerobic cobaltochelatase (EC: 6.6.1.2) catalyzing cobalt insertion in the corrin ring;
j) CobS subunit of aerobic cobaltochelatase (EC: 6.6.1.2) catalyzing cobalt insertion in the corrin ring;
k) CobT subunit of aerobic cobaltochelatase (EC: 6.6.1.2) catalyzing cobalt insertion in the corrin ring;
l) CobR 4-hydroxyphenylacetate 3-monooxygenase (EC: 1.14.14.9);
m) CobO corrinoid adenosyltransferase (EC: 2.5.1.17) synthesizing adenosylcobalamin from cob(II)yrinate a,c-diamide;
n) CobQ cobyric acid synthase (EC: 6.3.5.10) catalyzing aminidations of adenosylcobyrinic A,C-diamide;
o) BtuR corrinoid adenosyltransferase (EC: 2.5.1.17);
p) CobU bifunctional adenosylcobalamin biosynthesis 4-hydroxyphenylacetate 3-monooxygenase activity (EC: 2.7.1.156);
q) CobD threonine-phosphate decarboxylase (EC: 4.1.1.81) decarboxylating L-threonine-O-3-phosphate to yield (R)-1-amino-2-propanol O-2-phosphate, the precursor for the linkage between the nucleotide loop and the corrin ring in cobalamin;
r) CobC adenosylcobalamin/alpha-ribazole phosphatase (EC: 3.1.3.73) converting adenosylcobalamin 5′-phosphate to adenosylcobalamin;
s) CobT polypeptide having Nicotinate-nucleotide-dimethylbenzimidazole phosphoribosyltrans-ferase (EC: 2.4.2.21);
t) CobS adenosylcobinamide-GDP ribazoletransferase (EC: 2.7.8.26);
u) CbiB cobalamin biosynthase (EC: 6.3.1.10) converting cobyric acid into cobinamide;
v) PduX L-threonine kinase (EC: 2.7.1.177) converting L-threonine to L-threonine-O-3-phosphate;
w) CbiN cobalt transport protein;
x) ChiQ cobalt transport protein;
y) CbiM cobalt transport protein;
z) CbiO cobalt import ATP-binding protein (EC: 3.6.3 . . . ); and/or
aa) FeS cluster Transcription factor polypeptide (IscR) capable of regulating an operon [isc operon] producing a FeS cluster co-factor.

In some embodiments of the B₁₂ pathway the
CobG precorrin-3B synthase may have at least 70%, such at least 75%, such as at least 80%, such as at least 90%, such as at least 95%, such as at least 99%, such as 100% identity to the CobG precorrin-3B synthase comprised in SEQ ID NO: 157, 158, 159, 160, 161, 162, 163, 164, 165, 166. or 167;
the Cobl may have at least 70%, such at least 75%, such as at least 80%, such as at least 90%, such as at least 95%, such as at least 99%, such as 100% identity to the Cobl comprised in SEQ ID NO: 168;
the CobM may have at least 70%, such at least 75%, such as at least 80%, such as at least 90%, such as at least 95%, such as at least 99%, such as 100% identity to the CobM comprised in SEQ ID NO: 169;
the CobF may have at least 70%, such at least 75%, such as at least 80%, such as at least 90%, such as at least 95%, such as at least 99%, such as 100% identity to the CobF comprised in SEQ ID NO: 170;
the CobK may have at least 70%, such at least 75%, such as at least 80%, such as at least 90%, such as at least 95%, such as at least 99%, such as 100% identity to the CobK comprised in SEQ ID NO: 171;
the CobH may have at least 70%, such at least 75%, such as at least 80%, such as at least 90%, such as at least 95%, such as at least 99%, such as 100% identity to the CobH comprised in SEQ ID NO: 172;
the CobL may have at least 70%, such at least 75%, such as at least 80%, such as at least 90%, such as at least 95%, such as at least 99%, such as 100% identity to the CobL comprised in SEQ ID NO: 173;
the CobJ may have at least 70%, such at least 75%, such as at least 80%, such as at least 90%, such as at least 95%, such as at least 99%, such as 100% identity to the CobJ comprised in SEQ ID NO: 174;
the CobN may have at least 70%, such at least 75%, such as at least 80%, such as at least 90%, such as at least 95%, such as at least 99%, such as 100% identity to the CobN comprised in SEQ ID NO: 175;
the CobS may have at least 70%, such at least 75%, such as at least 80%, such as at least 90%, such as at least 95%, such as at least 99%, such as 100% identity to the CobS comprised in SEQ ID NO: 176;
the CobT may have at least 70%, such at least 75%, such as at least 80%, such as at least 90%, such as at least 95%, such as at least 99%, such as 100% identity to the CobT comprised in SEQ ID NO: 177;
the CobR may have at least 70%, such at least 75%, such as at least 80%, such as at least 90%, such as at least 95%, such as at least 99%, such as 100% identity to the CobR comprised in SEQ ID NO: 178;
the CobO may have at least 70%, such at least 75%, such as at least 80%, such as at least 90%, such as at least 95%, such as at least 99%, such as 100% identity to the CobO comprised in SEQ ID NO: 179;
the CobQ may have at least 70%, such at least 75%, such as at least 80%, such as at least 90%, such as at least 95%, such as at least 99%, such as 100% identity to the CobQ comprised in SEQ ID NO: 180;
the BtuR may have at least 70%, such at least 75%, such as at least 80%, such as at least 90%, such as at least 95%, such as at least 99%, such as 100% identity to the BtuR comprised in SEQ ID NO: 181;
the CobU may have at least 70%, such at least 75%, such as at least 80%, such as at least 90%, such as at least 95%, such as at least 99%, such as 100% identity to the CobU comprised in SEQ ID NO: 182;
the CobD may have at least 70%, such at least 75%, such as at least 80%, such as at least 90%, such as at least 95%, such as at least 99%, such as 100% identity to the CobD comprised in SEQ ID NO: 183;
the CobC may have at least 70%, such at least 75%, such as at least 80%, such as at least 90%, such as at least 95%, such as at least 99%, such as 100% identity to the CobC comprised in SEQ ID NO: 184;
the CobT may have at least 70%, such at least 75%, such as at least 80%, such as at least 90%, such as at least 95%, such as at least 99%, such as 100% identity to the CobT comprised in SEQ ID NO: 185;
the CobS may have at least 70%, such at least 75%, such as at least 80%, such as at least 90%, such as at least 95%, such as at least 99%, such as 100% identity to the CobS comprised in SEQ ID NO: 186;
the CbiB may have at least 70%, such at least 75%, such as at least 80%, such as at least 90%, such as at least 95%, such as at least 99%, such as 100% identity to the CbiB comprised in SEQ ID NO: 187;
the PduX may have at least 70%, such at least 75%, such as at least 80%, such as at least 90%, such as at least 95%, such as at least 99%, such as 100% identity to the PduX comprised in SEQ ID NO: 188;
the CbiN may have at least 70%, such at least 75%, such as at least 80%, such as at least 90%, such as at least 95%, such as at least 99%, such as 100% identity to the CbiN comprised in SEQ ID NO: 189;
the CbiQ may have at least 70%, such at least 75%, such as at least 80%, such as at least 90%, such as at least 95%, such as at least 99%, such as 100% identity to the CbiQ comprised in SEQ ID NO: 190;
the CbiM may have at least 70%, such at least 75%, such as at least 80%, such as at least 90%, such as at least 95%, such as at least 99%, such as 100% identity to the CbiM comprised in SEQ ID NO: 191;
the CbiO may have at least 70%, such at least 75%, such as at least 80%, such as at least 90%, such as at least 95%, such as at least 99%, such as 100% identity to the CbiO comprised in SEQ ID NO: 192; and/or
the IscR may have at least 70%, such at least 75%, such as at least 80%, such as at least 90%, such as at least 95%, such as at least 99%, such as 100% identity to the IscR comprised in SEQ ID NO: 77, 79, 81, 83, 85, 87, 89, 91, 93 or 95.

Where the gene(s) encoding the CobG polypeptide, precorrin-3B synthase, together with one or more additional polypeptides that catalyze additional steps in the Cob pathway are transgenes, these are preferably located in the genome of the genetically modified host cell, either integrated into the cell chromosome or on a self-replicating plasmid. The transgene encoding CobG and one or more of the transgenes Cob pathway enzymes may be present in the genome within one or more operons. The promoter driving expression of the transgene encoding CobG and one or more additional transgenes is preferably a non-native promoter, which may be a heterologous constitutive-promoter or an inducible-promoter. Suitable constitutive promoters include apFab family promoters of SEQ ID NO: 138 while a suitable inducible promoter includes pBad (arabinose-inducible) SEQ ID NO: 134 and lac promoter lac p, which is regulated by repressor lacl SEQ ID NO: 135. Suitable terminators include members of the apFAB terminator family including SEQ ID NO: 139. The selected promoter and terminator may be operably linked to the respective gene, either to provide individual gene regulation or for regulation of an operon.

B₁₂ cobalamin can be produced using genetically modified host cells such as described in example 5 of WO2020148351 further modified as described herein.

### B5 Branch

In other embodiments the vitamin B compound is vitamin B₅ or pantotheonate, and the host cell disclosed herein further comprises an operative metabolic pathway comprising one or more native or heterologous pathway elements producing vitamin B₅. In this context the term “pathway elements” include any step or element relevant for the functioning of the pathway, including proteins, polypeptides, peptides, enzymes, co-factors or the like. Host cells described herein genetically modified to lack or down-regulate CRP-cAMP complex and further modified with a transgene or up-regulated native IlvD gene encoding a dihydroxy-acid dehydratase (EC: 4.2.1.9) and optionally additional transgenes or up-regulated native genes encoding polypeptides PanB, PanE and PanC have an enhanced flux through the B₅ pathway, and enhanced production of pantothenic acid.

Accordingly, in some embodiments the pathway elements for producing vitamin B₅ can include one or more pathway elements are selected from:
a) IlvD dihydroxy-acid dehydratase (EC: 4.2.1.9);
b) PanB 3-methyl-2-oxobutanoate hydroxymethyltransferase;
c) PanE 2-dehydropantoate 2-reductase;
d) PanC Pantothenate synthetase; and/or
e) FeS cluster Transcription factor polypeptide (IscR) capable of regulating an operon [isc operon] producing a FeS cluster co-factor.

In some embodiments of the B₅ pathway
the IlvD dihydroxy-acid dehydratase may have at least 70%, such at least 75%, such as at least 80%, such as at least 90%, such as at least 95%, such as at least 99%, such as 100% identity to the IlvD dihydroxy-acid dehydratase comprised in SEQ ID NO: 194, 195, 196, 197, 198, 199, 200, 201, 202, 203 or 204;
the PanB 3-methyl-2-oxobutanoate hydroxymethyltransferase may have at least 70%, such at least 75%, such as at least 80%, such as at least 90%, such as at least 95%, such as at least 99%, such as 100% identity to the IlvD dihydroxy-acid dehydratase comprised in SEQ ID NO: 205;
the PanE 2-dehydropantoate 2-reductase may have at least 70%, such at least 75%, such as at least 80%, such as at least 90%, such as at least 95%, such as at least 99%, such as 100% identity to the IlvD dihydroxy-acid dehydratase comprised in SEQ ID NO: 206; and/or
the PanC Pantothenate synthetase may have at least 70%, such at least 75%, such as at least 80%, such as at least 90%, such as at least 95%, such as at least 99%, such as 100% identity to the IlvD dihydroxy-acid dehydratase comprised in SEQ ID NO: 207; and/or
the IscR may have at least 70%, such at least 75%, such as at least 80%, such as at least 90%, such as at least 95%, such as at least 99%, such as 100% identity to the IscR comprised in SEQ ID NO: 77, 79, 81, 83, 85, 87, 89, 91, 93 or 95.

Where the gene encoding the IIvD dihydroxy-acid dehydratase (eg. SEQ ID NO: 194), together with one or more additional polypeptides that catalyze additional steps in the pantothenic acid pathway (encoding PanB, PanE and PanC) are transgenes, they are located in the genome of the genetically modified host cell, either integrated into the cell chromosome or on a self-replicating plasmid. The transgene encoding IlvD and one or more of the transgenes encoding panBEC pathway enzymes may be present in the genome within one or more operons.

The promoter driving expression of the transgene encoding IlvD and one or more additional transgenes is preferably a non-native promoter, which may be a heterologous constitutive-promoter or an inducible-promoter. When the promoter is a heterologous constitutive promoter, then a suitable promoter includes the apFab family SEQ ID NO: 138, while a suitable inducible promoter includes: pBad (arabinose inducible SEQ ID NO: 134 and Lacl SEQ ID NO: 135. Suitable terminators include members of the apFAB terminator family including SEQ ID NO: 139. The selected promoter and terminator may be operably linked to the respective gene, either to provide individual gene regulation or for regulation of an operon.

B₅ pantothenoate can be produced using genetically modified host cells such as described in example 6 of WO2020148351 further modified as described herein.

For any of the Vitamin B compound pathways the IscR factor may be a mutant polypeptide having has at least one amino acid substitution selected from the group consisting of L15X, C92X, C98X, C104X, and H107X; wherein X is any amino acid other than the corresponding amino acid residue in the native IscR comprised in SEQ ID NO: 17. The amino acid substitution in the mutant IscR polypeptide can particularly be selected from the group consisting of:
a) L15X, wherein X is any one of F, Y, M and W;
b) C92X, wherein X is any one of Y, A, M, F and W;
c) C98X, wherein X is any one of A, V, I, L, F and W;
d) C104X, wherein X is any one of AV, I, L, F and W; and
e) H107X; wherein X, is any one of A, Y, V, I, and L.

In the host cell one or more genes and/or polypeptides of the pathway for the vitamin B compound may be heterologous to the host cell and may be present in the host cell in more than one copy, such as least 2 copies, such as least 3 copies, such as least 4 copies. The heterologous genes of the pathway for the vitamin B compound may be inserted in a plasmid or integrated in a chromosome. The host cell may in some embodiments comprise a transporter molecule facilitating transport of a precursor for or a product of the pathway for the vitamin B compound and/or the host cell may be further genetically modified to provide an increased amount of a substrate consumed in the pathway for the vitamin B compound. In addition, the host cell may further be genetically modified to exhibit increased tolerance towards one or more substrates, intermediates, or products in the pathway for the vitamin B compound and/or one or more native or endogenous genes of the host cell are deleted, disrupted and/or attenuated. For example, where the vitamin B compound is B₃ complex and/or quinolate it is desirable to down-regulate the NAD salvage pathway for example by deleting, disrupting and/or attenuating the nadR and/or pncC genes thereby reducing NR consumption.

In a particular embodiment, the host cell may also be modified to overexpress one or more genes in the pathway for the vitamin B compound.

### Host Cells.

The host cell of the invention may be any host cell suitable for hosting and expressing the pathway for the vitamin B compound. Such cell may be a prokaryotic or eukaryotic cell. Suitable prokaryotic host cells can be of a genus selected from Escherichia, Bacillus, Brevibacterium, Burkholderia, Campylobacter, Corynebacterium, Serratia, Lactobacillus, Lactococcus, Acinetobacter, Acetobacter or Pseudomonas. Particularly useful prokaryotic host cells are of the genus Escherichia, Corynebacterium, Bacillus, Serratia, or Pseudomonas, such as the species Escherichia coli, Corynebacterium glutamicum, Bacillus subtilis, Serratia marcescens, Pseudomonas putida and/or Pseudomonas mutabilis. Useful eukaryotic host cells include mammalian, insect, plant, fungal or archaeal cells. Among the eukaryotic host cells fungal cells of the genuses Saccharomyces, Kluveromyces, Candida, Pichia, Debaromyces, Hansenula, Yarrowia, Zygosaccharomyces, Schizosaccharomyces and Ashbya are particularly useful, such as the species Kluyveromyces lactis, Saccharomyces carlsbergensis, Saccharomyces cerevisiae, Saccharomyces diastaticus, Saccharomyces douglasii, Saccharomyces kluyveri, Saccharomyces bensis, Saccharomyces oviformis, Yarrowia lipolytica, Pichia pastoris or Ashbya gossypii.

### Mutated CRP, CRR and CyaA

The invention also provides mutated CRP, CRR and/or CyaA polypeptides. In one embodiment a mutated CRP polypeptide is provided which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the CRP comprised in SEQ ID NO: 39 or 97 and comprises one or more mutations in positions corresponding to positions T12, D138, T146, F69, R82, V139, G57, K58, E59, M60, 161, L62, S63, G72, E73, L74, R83, S84, T128, S129, A136, F137, Q171, E172, 1173, G174, Q175, 1176, V177, G178, C179, S180, R181, E182, T183, V184, G185, R186 of SEQ ID NO: 39 or 97.

In another embodiment a mutated CRR polypeptide is provided which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the CRR comprised in SEQ ID NO: 41 or 99 and comprises one or more mutations in positions corresponding to H76, H91 of SEQ ID NO: 41 or 99.

In another embodiment a mutated CyaA polypeptide is provided which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the CyaA comprised in SEQ ID NO: 43 or 101 and comprises one or more mutations in positions corresponding to G60, K59, L63, T65, R188, G195, K196, R192, S103, S113, D114, D116, W118, E185, T189, K260, K264, K332, W200, D300 of SEQ ID NO: 43 or 101.

### Polynucleotide Constructs and Expression Vectors

Also provided for herein are polynucleotide constructs harboring gene(s) encoding native or mutated CRP, CyaA or CRR operably linked to one or more control sequences, said polynucleotide constructs comprising mutations to delete, disrupt, and/or an attenuate the gene transcription or translation or the activity and/or function of the encoded CRP, CyaA or CRR. Alternatively, the polynucleotide constructs provided for herein harbor gene(s) encoding native or mutated CpdA or CadD operably linked to one or more control sequences, said polynucleotide constructs comprising mutations to increase the gene transcription or translation of the cAMP degrading activity of the encoded CpdA or CadD.

The control sequences direct the expression of the encoded CRP, CyaA, CRR, CpdA and/or CadD in the host cell harboring the polynucleotide construct. Conditions for the expression should be compatible with the control sequences. The control sequence may be heterologous or native to the gene(s) encoding the CRP, CyaA, CRR, CpdA or CadD and/or to the host cell. In some embodiments both the control sequence and the gene(s) encoding the CRP, CyaA, CRR, CpdA and/or CadD are heterologous to the host cell and optionally also to each other. In one embodiment the polynucleotide construct is an expression vector, comprising the gene(s) encoding the CRP, CyaA, CRR, CpdA and/or CadD operably linked to the one or more control sequences.

Polynucleotides may be manipulated in a variety of ways to modify expression of the CRP, CyaA, CRR, CpdA or CadD. Manipulation of the polynucleotide prior to its insertion into an expression vector may be desirable or necessary depending on the expression vector. The techniques for modifying polynucleotides utilizing recombinant DNA methods are well known in the art.

The control sequence may be a promoter, which is a polynucleotide that is recognized by a host cell for expression of a polynucleotide. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell. The promoter may also be an inducible promoter. Selecting a suitable promoter for expression in yeast is well-known and is well understood by persons skilled in the art.

The control sequence may also be a transcription terminator, which is recognized by a host cell to terminate transcription. The terminator is operably linked to the 3′-terminus of the polynucleotide encoding the polypeptide. Any terminator that is functional in the host cell may be used.

The control sequence may also be an mRNA stabilizer region downstream of a promoter and upstream of the coding sequence of a gene which increases expression of the gene.

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

It may also be desirable to add regulatory sequences that regulate expression of the CRP, CyaA CRR, CpdA and/or CadD relative to the growth of the host cell. Examples of regulatory systems are those that cause expression of the gene to be turned on or off in response to a chemical or physical stimulus, including the presence of a regulatory compound.

Various nucleotide sequences in addition to the polynucleotide construct of the invention may be joined together to produce a recombinant expression vector, which may include one or more convenient restriction sites to allow for insertion or substitution of the polynucleotide sequence encoding the CRP, CyaA, CRR, CpdA and/or CadD at such sites. The recombinant expression vector may be any vector (e.g., a plasmid or virus or chromosomal) that can be conveniently subjected to recombinant DNA procedures and can bring about expression of the CRP, CyaA, CRR, CpdA and/or CadD. The choice of the vector will typically depend on the compatibility of the vector with the host cell into which the vector is to be introduced. The vector may be an autonomously replicating vector, i.e., a vector that exists as an extrachromosomal entity, the replication of which is independent of chromosomal replication, e.g., a plasmid (linear or closed circular plasmid), an extrachromosomal element, a mini-chromosome, or an artificial chromosome. The vector may contain any means for assuring self-replication. Alternatively, the vector may, when introduced into the host cell, integrate into the genome, and replicate together with the chromosome(s) into which it has been integrated. Furthermore, a single vector or plasmid or two or more vectors or plasmids that together contain the total DNA to be introduced into the genome of the host cell, or a transposon, may be used.

The vector may contain one or more selectable markers that permit easy selection of transformed, transfected, transduced, or the like cells. A selectable marker is a gene from which the product provides for biocide or viral resistance, resistance to heavy metals, prototrophy to auxotrophs, and the like.

The vector may further contain element(s) that permits integration of the vector into genome (being a vector in itself) of the host cell or permits autonomous replication of the vector in the cell independent of the genome. Alternatively, the vector may contain additional polynucleotides for directing integration by homologous recombination into the genome of the host cell at precise location(s) in the chromosome(s). To increase the likelihood of integration at a precise location, the integrational elements should contain a sufficient number of nucleic acids, such as 100 to 10,000 base pairs, such as 400 to 10,000 base pairs, and such as 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” refers to a polynucleotide that enables a plasmid or vector to replicate in vivo.

As mentioned, supra, more than one copy of a gene encoding pathway elements for vitamin B compounds may be inserted into a host cell to increase production of the vitamin B compound. An increase in the gene copy number can be obtained by integrating one or more additional copies of a gene into the host cell genome or by including an amplifiable selectable marker gene with the gene, so that cells containing amplified copies of the selectable marker gene—and thereby additional copies of the polynucleotide—can be selected by cultivating the cells in the presence of the appropriate selectable agent. The procedures used to ligate the elements described above to construct the recombinant expression vectors of the present disclosure are well known to one skilled in the art (see, e.g., Sambrook et al., 1989, supra).

In a separate embodiment the host cell comprising the polynucleotide constructs and/or vectors as disclosed herein.

### Cultures

Further provided for herein are cell cultures comprising the genetically modified host cells of the invention and a growth medium. Suitable growth mediums for relevant prokaryotic or eukaryotic host cells are widely known in the art.

### Methods of Producing Compounds of the Invention.

The invention also provides a method for producing vitamin B compounds (such as biotin or thiamine) comprising
a) culturing the cell culture of the invention at conditions allowing the host cells to produce the vitamin B compound; and
b) optionally recovering and/or isolating the vitamin B compound.

The cell culture can be cultivated in a nutrient medium and at conditions suitable for production of the vitamin B compounds of the invention and/or for propagating cell count using methods known in the art. For example, the culture may be cultivated by shake flask cultivation, or small-scale or large-scale fermentation (including continuous, batch, fed-batch, or solid-state fermentations) in laboratory or industrial fermenters in a suitable medium and under conditions allowing the host cells to grow and/or propagate, optionally to be recovered and/or isolated.

The cultivation can take place in a suitable nutrient medium comprising carbon and nitrogen sources and inorganic salts, using procedures known in the art. Suitable media are available from commercial suppliers or may be prepared according to published recipes (e.g., from catalogues of the American Type Culture Collection). The selection of the appropriate medium may be based on the choice of host cell and/or based on the regulatory requirements for the host cell. Such media are available in the art. The medium may, if desired, contain additional components favouring the transformed expression hosts over other potentially contaminating microorganisms. Accordingly, in an embodiment a suitable nutrient medium can include one or more of (i) trace metals; (ii) vitamins; (iii) salts (such as salts of phosphate, magnesium, potassium, zinc, iron); (iv) nitrogen sources (such as YNB, ammonium sulfate, urea, yeast extracts, ammonium nitrate, ammonium chloride, malt extract, peptone and/or amino acids); (v) carbon source (such as dextrose, sucrose, glycerol, glucose, maltose, molasses, starch, cellulose, xylan, pectin, lignocellolytic biomass hydrolysate, and/or acetate); (vi) nucleobases; (vii) aminoglycosides; and/or (viii) antibiotics (such as G418 and hygromycin B).

The cultivation of the host cell may be performed over a period of from about 0.5 to about 30 days. The cultivation process may be a batch process, continuous or fed-batch process, suitably performed at a temperature in the range of 0-100° C. or 0-80° C., for example, from about 0° C. to about 50° C. and/or at a pH, for example, from about 2 to about 10. Preferred fermentation conditions are a temperature in the range of from about 25° C. to about 55° C. and at a pH of from about 3 to about 9. The appropriate conditions are usually selected based on the choice of host cell. Accordingly, in some embodiments the method of the invention comprising one or more elements selected from:
a) culturing the cell culture under aerobic or anaerobic conditions
b) cultivating the host cells under mixing;
c) cultivating the host cells at a temperature of between 25° C. to 50° C.;
d) cultivating the host cells at a pH of between 3-9; and
e) cultivating the host cells for between 10 hours to 120 days.

The cell culture of the invention may be recovered and or isolated using methods known in the art. For example, the compound(s) may be recovered from the nutrient medium by conventional procedures including, but not limited to, centrifugation, filtration, spray-drying, or lyophilization. In a particular embodiment the method includes a recovery and/or isolation step comprising separating a liquid phase of the cell culture from a solid phase of the cell culture to obtain a supernatant comprising the vitamin B compound and subjecting the supernatant to one or more steps selected from:
a) contacting the supernatant with one or more adsorbent resins to obtain at least a portion of the produced vitamin B compound, then optionally recovering the vitamin B compound from the resin in a concentrated solution prior to isolation of the vitamin B compound by crystallisation or solvent evaporation;
b) contacting the supernatant with one or more ion exchange or reversed-phase chromatography columns to obtain at least a portion of the vitamin B compound, then optionally recovering the vitamin B compound from the resin in a concentrated solution prior to isolation of the vitamin B compound by crystallisation or solvent evaporation;
c) extracting the vitamin B compound from the supernatant, such as by liquid-liquid extraction into an immiscible solvent, then optionally isolating the vitamin B compound by crystallisation or solvent evaporation; and
   thereby recovering and/or isolating the vitamin B compound.

The yield of vitamin B compound, such as biotin or thiamine provided by the method of the invention is typically higher than when producing Vitamin B compounds by methods employing host cells without reduced CRP-cAMP complex formation and/or increased degradation and/or decreased binding of cAMP, in some embodiments at least 10% higher such as at least 50%, such as at least 100%, such as least 150%, such as at least 200% higher.

The method of the invention may further comprise one or more steps of mixing the vitamin B compound with one or more carriers, agents, adjuvants, additives and/or excipients, optionally pharmaceutical grade carriers, agents, adjuvants, additives and/or excipients.

The method of the invention may further comprise one or more in vitro steps in the process of producing the vitamin B compound. It may also comprise one or more in vivo steps performed in another cell than the host cell of the invention. For example, precursors for and/or intermediates in the pathway for the vitamin B compound may be produced in another cell and isolated therefrom and then fed to a cell culture of the invention for conversion into the vitamin B compound. Accordingly, in one embodiment the method of the invention further comprises feeding one or more exogenous vitamin B precursors to the host cell culture, such as O-methylpimeloyl-acyl carrier protein, pimeloyl-acyl carrier protein, KAPA, DAPA, DTB and/or pimelate for biotin production, whereas precursors such as thiazole, or HMP would be suitable for production of thiamine, TMP or TPP.

### Fermentation Composition

The disclosure also describes a fermentation composition comprising the cell culture of the invention and the vitamin B compound-either comprised in the cells or in the medium. In the fermentation composition the genetically modified host cells may be wholly or partially lysed and/or disintegrated. In some embodiments at least 50%, such as at least 75%, such as at least 95%, such as at least 99% of the genetically modified host cells in the fermentation composition are lysed and/or disintegrated. Further, in the fermentation composition of the invention at least 50%, such as at least 75%, such as at least 95%, such as at least 99% of solid cellular material may have been separated and/or removed from a liquid phase of the fermentation composition.

The fermentation composition may further comprise one or more compounds of
a) precursor or products of the operative metabolic pathway producing the vitamin B compound;
b) supplemental nutrients comprising; and
   wherein the concentration of the vitamin B compound is at least 1 mg/L composition. In particular the fermentation composition can comprise a concentration of vitamin B compound of at least 5 mg/kg, such as at least 10 mg/kg, such as at least 20 mg/kg, such as at least 50 mg/kg, such as at least 100 mg/kg, such as at least 500 mg/kg, such as at least 1000 mg/kg, such as at least 5000 mg/kg, such as at least 10000 mg/kg, such as at least 50000 mg/kg.

Suitable supplemental nutrients can include one or more of (i) trace metals; (ii) vitamins; (iii) salts (such as salts of phosphate, magnesium, potassium, zinc, iron); (iv) nitrogen sources (such as YNB, ammonium sulfate, urea, yeast extracts, ammonium nitrate, ammonium chloride, malt extract, peptone and/or amino acids); (v) carbon source (such as dextrose, sucrose, glycerol, glucose, maltose, molasses, starch, cellulose, xylan, pectin, lignocellolytic biomass hydrolysate, and/or acetate); (vi) nucleobases; (vii) aminoglycosides; and/or (viii) antibiotics (such as G418, hygromycin B, spectinomycin and/or Kanamycin).

### Compositions and Use

The invention also provides a composition comprising the fermentation composition of the invention and one or more carriers, agents, adjuvants, additives and/or excipients and at least trace amounts of one or more metabolites of the cell culture, optionally signature metabolites for the genetically modified host cell. Suitable carriers, agents, adjuvants, additives and/or excipients includes formulation additives, stabilising agent, fillers and the like. The composition and the one or more carriers, agents, adjuvants, additives and/or excipients can suitably be formulated into in a dry solid form, e.g., by using methods known in the art, such as spray drying, spray cooling, lyophilization, flash freezing, granulation, microgranulation, encapsulation or microencapsulation. The composition and the one or more carriers, agents, adjuvants, additives and/or excipients can also be formulated into a liquid stabilized form using methods known in the art, such as adding to the fermentation composition one or more stabilizers such as sugars and/or polyols (e.g., sugar alcohols) and/or organic acids (e.g., lactic acid).

The composition of the invention may be further refined into a pharmaceutical preparation, a dietary supplement, a cosmetic, a food/flavor preparation, a feed preparation and/or an analytical or diagnostic reagent optionally using one or more steps of the methods described herein for producing the vitamin B compound including mixing the vitamin B compound with one or more pharmaceutical grade carriers, agents, adjuvants, additives and/or excipients. In one embodiment, the pharmaceutical composition is a pharmaceutical preparation obtainable from the method of the invention. The pharmaceutical preparation may be a dry preparation, optionally in the form of a powder, tablet, capsule, hard chewable and or soft lozenge or a gum. Alternatively, the pharmaceutical preparation may in form of a liquid pharmaceutical solution. Such pharmaceutical preparations may be used as a medicament in a method for treating and/or relieving a disease and/or medical condition, in particular in a mammal, in particular for use in the treatment of a nutritional deficiency. Accordingly, the invention further provides a method for preventing, treating and/or relieving a disease and/or medical condition comprising administering a therapeutically effective amount of the pharmaceutical composition of the invention to a mammal in need of treatment and/or relief. Diseases and/or medical conditions treatable or relievable by the pharmaceutical composition includes but is not limited to diseases and/or medical conditions associated with lacking or insufficient bodily intake of vitamin B compounds. The pharmaceutical preparation can be administered parenterally, such as topically, epicutaneously, sublingually, buccally, nasally, intradermally, intravenously, and/or intramuscularly. The pharmaceutical composition can also be administered enterally via the gastrointestinal tract.

The invention also provides a kit of parts comprising
a) the genetically modified host cell as described herein; and/or
b) instructions for use of the genetically modified host cell; and/or
c) the nucleic acid construct as described herein; and/or
d) instructions for use of the nucleic acid construct; and/or
e) a host cell which can be genetically modified using the methods described herein.

In some embodiments, the kit comprises a genetically modified cell capable of producing a vitamin B compound, wherein the genetically modified cell expresses pathway enzymes producing the vitamin B compound. In one embodiment the Vitamin B compound is Biotin (B₇) and the genetically modified cell expresses Biotin synthase (BioB) and optionally one or more biotin pathway enzymes or factors selected from BioC, BioH, BioF, BioA, Biok, BioD, Biol, BioW and IscR. In other embodiments the genetically modified cell expresses thiamine synthase (ThiC) and optionally one or more thiamine pathway enzymes or factors selected from ThiD, ThiF, This, ThiH, ThiG, ThiM, ThiE, TMP phosphatase, Thik, ThiL, Thil, IscR and ThiO. In some embodiments, the kit comprises a genetically modified cell capable of producing riboflavin (B₂). In some embodiments, the kit comprises a genetically modified cell capable of producing niacin (B₃). In some embodiments, the kit comprises a genetically modified cell capable of producing pantothenic acid (B₅). In some embodiments, the kit comprises a genetically modified cell capable of producing pyridoxine (B₆). In some embodiments, the kit comprises a genetically modified cell capable of producing folate (B₉). In some embodiments, the kit comprises a genetically modified cell capable of producing cobalamin (B₁₂).

### SEQUENCE LISTING

The present application contains a Sequence Listing prepared in PatentIn included below but also submitted electronically in ST26 format which is hereby incorporated by reference in its entirety.

TABLE A
SEQ ID NO: 1: Protein sequence of BioC
SEQ ID NO: 2: DNA Sequence encoding BioC
SEQ ID NO: 3: Protein sequence of BioH
SEQ ID NO: 4: DNA Sequence encoding BioH
SEQ ID NO: 5: Protein sequence of Biof
SEQ ID NO: 6: DNA Sequence encoding Biof
SEQ ID NO: 7: Protein sequence of BioA
SEQ ID NO: 8: DNA Sequence encoding BioA
SEQ ID NO: 9: Protein sequence of BioK
SEQ ID NO: 10: DNA Sequence encoding BioK
SEQ ID NO: 11: Protein sequence of BioD
SEQ ID NO: 12: DNA Sequence encoding BioD
SEQ ID NO: 13: Protein sequence of BioI
SEQ ID NO: 14: DNA Sequence encoding BioI
SEQ ID NO: 15: Protein sequence of BioW
SEQ ID NO: 16: DNA Sequence encoding BioW
SEQ ID NO: 17: Protein sequence of IscR
SEQ ID NO: 18: DNA Sequence encoding IscR
SEQ ID NO: 19: Protein sequence of IscR
SEQ ID NO: 20: DNA Sequence encoding IscR
SEQ ID NO: 21: Protein sequence of IscR
SEQ ID NO: 22: DNA Sequence encoding IscR
SEQ ID NO: 23: Protein sequence of IscR
SEQ ID NO: 24: DNA Sequence encoding IscR
SEQ ID NO: 25: Protein sequence of IscR
SEQ ID NO: 26: DNA Sequence encoding IscR
SEQ ID NO: 27: Protein sequence of IscR
SEQ ID NO: 28: DNA Sequence encoding IscR
SEQ ID NO: 29: Protein sequence of IscR
SEQ ID NO: 30: DNA Sequence encoding IscR
SEQ ID NO: 31: Protein sequence of IscR
SEQ ID NO: 32: DNA Sequence encoding IscR
SEQ ID NO: 33: Protein sequence of IscR
SEQ ID NO: 34: DNA Sequence encoding IscR
SEQ ID NO: 35: Protein sequence of IscR
SEQ ID NO: 36: DNA Sequence encoding IscR
SEQ ID NO: 37: Protein sequence of BioB
SEQ ID NO: 38: DNA Sequence encoding BioB
SEQ ID NO: 39: Protein sequence of CRP
SEQ ID NO: 40: DNA Sequence encoding CRP
SEQ ID NO: 41: Protein sequence of CRR
SEQ ID NO: 42: DNA Sequence encoding CRR
SEQ ID NO: 43: Protein sequence of CyaA
SEQ ID NO: 44: DNA Sequence encoding CyaA
SEQ ID NO: 45: Protein sequence of CdpA
SEQ ID NO: 46: DNA Sequence encoding CdpA
SEQ ID NO: 47: Protein sequence of CadD
SEQ ID NO: 48: DNA Sequence encoding CadD
SEQ ID NO: 49: DNA Sequence encoding T5LacO repressed promoter
SEQ ID NO: 50: DNA Sequence encoding Promoter ApFAB346
SEQ ID NO: 51: Protein sequence of ThiC
SEQ ID NO: 52: DNA sequence encoding ThiC
SEQ ID NO: 53: Protein sequence of ThiD
SEQ ID NO: 54: DNA sequence encoding ThiD
SEQ ID NO: 55: Protein sequence of ThiF
SEQ ID NO: 56: DNA sequence encoding ThiF
SEQ ID NO: 57: Protein sequence of ThiS
SEQ ID NO: 58: DNA sequence encoding ThiS
SEQ ID NO: 59: Protein sequence of ThiH
SEQ ID NO: 60: DNA sequence encoding ThiH
SEQ ID NO: 61: Protein sequence of ThiG
SEQ ID NO: 62: DNA sequence encoding ThiG
SEQ ID NO: 63: Protein sequence of ThiM
SEQ ID NO: 64: DNA sequence encoding ThiM
SEQ ID NO: 65: Protein sequence of TMP Phosphatase (ATH2)
SEQ ID NO: 66: DNA sequence encoding TMP Phosphatase (ATH2)
SEQ ID NO: 67: Protein sequence of ThiK
SEQ ID NO: 68: DNA sequence encoding ThiK
SEQ ID NO: 69: Protein sequence of ThiL
SEQ ID NO: 70: DNA sequence encoding ThiL
SEQ ID NO: 71: Protein sequence of ThiE
SEQ ID NO: 72: DNA sequence encoding ThiE
SEQ ID NO: 73: Protein sequence of ThiI
SEQ ID NO: 74: DNA sequence encoding ThiI
SEQ ID NO: 75: Protein sequence of IscS DNA
SEQ ID NO: 76: DNA sequence encoding IscS DNA
SEQ ID NO: 77: Protein sequence of IscR Protein
SEQ ID NO: 78: DNA sequence encoding IscR DNA
SEQ ID NO: 79: Protein sequence of IscR Protein
SEQ ID NO: 80: DNA sequence encoding IscR DNA
SEQ ID NO: 81: Protein sequence of IscR Protein
SEQ ID NO: 82: DNA sequence encoding IscR DNA
SEQ ID NO: 83: Protein sequence of IscR Protein
SEQ ID NO: 84: DNA sequence encoding IscR DNA
SEQ ID NO: 85: Protein sequence of IscR Protein
SEQ ID NO: 86: DNA sequence encoding IscR DNA
SEQ ID NO: 87: Protein sequence of IscR Protein
SEQ ID NO: 88: DNA sequence encoding IscR DNA
SEQ ID NO: 89: Protein sequence of IscR Protein
SEQ ID NO: 90: DNA sequence encoding IscR DNA
SEQ ID NO: 91: Protein sequence of IscR Protein
SEQ ID NO: 92: DNA sequence encoding IscR DNA
SEQ ID NO: 93: Protein sequence of IscR Protein
SEQ ID NO: 94: DNA sequence encoding IscR DNA
SEQ ID NO: 95: Protein sequence of IscR Protein
SEQ ID NO: 96: DNA sequence encoding IscR DNA
SEQ ID NO: 97: Protein sequence of CRP
SEQ ID NO: 98: DNA sequence encoding CRP
SEQ ID NO: 99: Protein sequence of CRR
SEQ ID NO: 100: DNA sequence encoding CRR
SEQ ID NO: 101: Protein sequence of CyaA
SEQ ID NO: 102: DNA sequence encoding CyaA
SEQ ID NO: 103: Protein sequence of CdpA
SEQ ID NO: 104: DNA sequence encoding CdpA
SEQ ID NO: 105: Protein sequence of CadD
SEQ ID NO: 106: DNA sequence encoding CadD
SEQ ID NO: 107: DNA sequence encoding T5LacO repressed promoter
SEQ ID NO: 108: DNA sequence encoding Promoter ApFAB346
SEQ ID NO: 109: Protein sequence of ThiO
SEQ ID NO: 110: DNA sequence encoding ThiO
SEQ ID NO: 111: Protein sequence of ThiO
SEQ ID NO: 112: DNA sequence encoding ThiO
SEQ ID NO: 113: Protein sequence of ThiO
SEQ ID NO: 114: DNA sequence encoding ThiO
SEQ ID NO: 115: Protein sequence of ThiO
SEQ ID NO: 116: DNA sequence encoding ThiO
SEQ ID NO: 117: DNA Oligo sequence
SEQ ID NO: 118: DNA Oligo sequence
SEQ ID NO: 119: DNA Oligo sequence
SEQ ID NO: 120: DNA Oligo sequence
SEQ ID NO: 121: DNA Oligo sequence
SEQ ID NO: 122: DNA Oligo sequence
SEQ ID NO: 123: DNA Oligo sequence
SEQ ID NO: 124: DNA Oligo sequence
SEQ ID NO: 125: DNA Oligo sequence
SEQ ID NO: 126: DNA Oligo sequence
SEQ ID NO: 127: DNA Oligo sequence
SEQ ID NO: 128: DNA Oligo sequence
SEQ ID NO: 129: DNA Oligo sequence
SEQ ID NO: 130: DNA Oligo sequence
SEQ ID NO: 131: skipped sequence
SEQ ID NO: 132: DNA sequence of FAB terminator
SEQ ID NO: 133: DNA sequence of T5 LacO repressed promoter
SEQ ID NO: 134: DNA sequence of pBad
SEQ ID NO: 135: DNA sequence of lacI repressor
SEQ ID NO: 136: DNA sequence of promoter apFAB309
SEQ ID NO: 137: DNA sequence of rrnB terminator
SEQ ID NO: 138: DNA sequence of apFab promoter family
SEQ ID NO: 139: DNA sequence of apFAB terminator
SEQ ID NO: 140: Protein sequence of NadA
SEQ ID NO: 141: Protein sequence of NadA
SEQ ID NO: 142: Protein sequence of NadA
SEQ ID NO: 143: Protein sequence of NadA
SEQ ID NO: 144: Protein sequence of NadA
SEQ ID NO: 145: Protein sequence of NadA
SEQ ID NO: 146: Protein sequence of NadA
SEQ ID NO: 147: Protein sequence of NadA
SEQ ID NO: 148: Protein sequence of NadA
SEQ ID NO: 149: Protein sequence of NadA
SEQ ID NO: 150: Protein sequence of NadA
SEQ ID NO: 151: Protein sequence of NadB
SEQ ID NO: 152: Protein sequence of NadE
SEQ ID NO: 153: Protein sequence of AphA
SEQ ID NO: 154: Protein sequence of pncA
SEQ ID NO: 155: Protein sequence of Chi (NMN)
SEQ ID NO: 156: Protein sequence of NadC
SEQ ID NO: 157: Protein sequence of CobG
SEQ ID NO: 158: Protein sequence of CobG
SEQ ID NO: 159: Protein sequence of CobG
SEQ ID NO: 160: Protein sequence of CobG
SEQ ID NO: 161: Protein sequence of CobG
SEQ ID NO: 162: Protein sequence of CobG
SEQ ID NO: 163: Protein sequence of CobG
SEQ ID NO: 164: Protein sequence of CobG
SEQ ID NO: 165: Protein sequence of CobG
SEQ ID NO: 166: Protein sequence of CobG
SEQ ID NO: 167: Protein sequence of CobG
SEQ ID NO: 168: Protein sequence of CobI
SEQ ID NO: 169: Protein sequence of CobM
SEQ ID NO: 170: Protein sequence of CobF
SEQ ID NO: 171: Protein sequence of CobK
SEQ ID NO: 172: Protein sequence of CobH
SEQ ID NO: 173: Protein sequence of CobL
SEQ ID NO: 174: Protein sequence of CobJ
SEQ ID NO: 175: Protein sequence of CobN
SEQ ID NO: 176: Protein sequence of CobS
SEQ ID NO: 177: Protein sequence of CobT
SEQ ID NO: 178: Protein sequence of CobR
SEQ ID NO: 179: Protein sequence of CobO
SEQ ID NO: 180: Protein sequence of CobQ
SEQ ID NO: 181: Protein sequence of BtuR
SEQ ID NO: 182: Protein sequence of CobU
SEQ ID NO: 183: Protein sequence of CobD
SEQ ID NO: 184: Protein sequence of CobC
SEQ ID NO: 185: Protein sequence of CobT
SEQ ID NO: 186: Protein sequence of CobS
SEQ ID NO: 187: Protein sequence of CbiB
SEQ ID NO: 188: Protein sequence of PduX
SEQ ID NO: 189: Protein sequence of CbiN
SEQ ID NO: 190: Protein sequence of CbiQ
SEQ ID NO: 191: Protein sequence of CbiM
SEQ ID NO: 192: Protein sequence of CbiO
SEQ ID NO: 193: Skipped sequence
SEQ ID NO: 194: Protein sequence of IlvD
SEQ ID NO: 195: Protein sequence of IlvD
SEQ ID NO: 196: Protein sequence of IlvD
SEQ ID NO: 197: Protein sequence of IlvD
SEQ ID NO: 198: Protein sequence of IlvD
SEQ ID NO: 199: Protein sequence of IlvD
SEQ ID NO: 200: Protein sequence of IlvD
SEQ ID NO: 201: Protein sequence of IlvD
SEQ ID NO: 202: Protein sequence of IlvD
SEQ ID NO: 203: Protein sequence of IlvD
SEQ ID NO: 204: Protein sequence of IlvD
SEQ ID NO: 205: Protein sequence of PanB
SEQ ID NO: 206: Protein sequence of PanE
SEQ ID NO: 207: Protein sequence of PanC

### EXAMPLES

### Materials

### Chemicals

Chemicals used herein, e.g., for buffers, media and substrates are commercial products of at least reagent grade.

mMOPS Medium
The minimal medium (mMOPS) used herein had the following composition in demineralized H₂O (dH₂O):

| | Concentration of |
|---|---|
| Compound | compound in medium - [g/L] |
| 3-(N-morpholino)propane | 8.3705 |
| sulfonic acid (MOPS) | |
| Tricine | 0.7167 |
| K₂HPO₄ | 0.2300 |
| NH₄Cl | 0.5082 |
| NaCl | 2.9220 |
| MgCl2 | 0.0050 |
| ZnSO₄•7H₂O | 0.00007 |
| CuSO₄•5H₂O | 0.00002 |
| MnCl2 | 0.0002 |
| FeSO4₃•7H₂O | 0.0029 |
| CoCl₂•6H₂O | 0.00007 |
| CaCl₂•2H₂O | 0.00007 |
| K₂SO₄ | 0.0481 |
| (NH₄)₆Mo₇O₂₄•4H₂O | 0.00004 |
| H₃BO₃ | 0.0003 |
| Calcium pantothenate | 0.0190 |
| P-aminobenzoic acid | 0.0055 |
| P-hydroxybenzoic acid | 0.0055 |
| 2,3-dihydroxybenzoic acid | 0.0062 |
| Biotin | 0.000002 |
| D-glucose | 2.0000 |

pH of the mMOPS medium was adjusted to 7.4±0.1 with NaOH or H₂SO₄.

### S Medium

The minimal screening medium (S medium) used herein had the following composition in dH2O:

| | Concentration of |
|---|---|
| Compound | compound in medium - [g/L] |
| 3-(N-morpholino)propane | 41.89 |
| sulfonic acid (MOPS) | |
| (NH₄)₂SO₄ | 10.00 |
| K₂HPO₄ | 3.00 |
| Na₃Citrate•2H₂O | 3.00 |
| Na₂SO₄ | 2.00 |
| NH₄Cl | 1.00 |
| MgSO₄•7H₂O | 0.50 |
| ZnSO₄•7H₂O | 0.0006 |
| CuSO₄•5H₂O | 0.0005 |
| MnSO₄•H₂O | 0.000 |
| FeCl₃•6H₂O | 0.0418 |
| Ethylenediaminetetraacetic | 0.0334 |
| acid (EDTA) disodium | |
| CoCl₂•6H₂O | 0.0006 |
| CaCl₂•2H₂O | 0.002 |
| Biotin | 0.000002 |
| D-glucose | 10.00 |

pH of the S medium was adjusted to 7.4±0.1 with NaOH or H₂SO₄

### LB Agar Medium

The LB agar medium used herein had the following composition in dH₂O:

| | Concentration of |
|---|---|
| Compound | compound in medium - [g/L] |
| LB Broth (Lennox) | 20.00 |
| Agar | 20.00 |

mMOPS Agar Medium
The mMOPS agar medium used herein had the following composition in mMOPS medium:

| | Concentration of |
|---|---|
| Compound | compound in medium - [g/L] |
| Desthiobiotin | 0.10 |
| Agar | 15.00 |

### B Medium

The fermentation batch medium (B medium) used herein had the following composition in dH2O:

| | Concentration of |
|---|---|
| Compound | compound in medium - [g/L] |
| (NH₄)₂SO₄ | 10.00 |
| K₂HPO₄ | 4.00 |
| KH₂PO₄ | 4.00 |
| Na₃Citrate•2H₂O | 3.00 |
| Na₂SO₄ | 2.00 |
| NH₄Cl | 1.00 |
| MgSO₄•7H₂O | 1.00 |
| ZnSO₄•7H₂O | 0.000825 |
| CuSO₄•5H₂O | 0.00075 |
| MnSO₄•H₂O | 0.00045 |
| FeCl₃•6H₂O | 0.0627 |
| CoCl₂•6H₂O | 0.0009 |
| CaCl₂•2H₂O | 0.003 |
| Boric acid | 0.001125 |
| Yeast extract | 10.00 |
| Biotin | 0.00002 |
| D-glucose | 10.00 |

### F Medium

The fermentation feed medium (F medium) used herein had the following composition in dH2O:

| | Concentration of |
|---|---|
| Compound | compound in medium - [g/L] |
| MgSO₄•7H₂O | 10.00 |
| FeCl₃•6H₂O | 0.270 |
| D-glucose | 550.00 |

### G Medium

The fermentation feed medium (F medium) used herein had the following composition in dH2O:

| | Concentration of compound |
|---|---|
| Compound | in medium - [g/L] |
| MgSO₄•7H₂O | 5.00 |
| D-glucose | 275.00 |

### Antibiotic Solution

The antibiotics solution used herein had the following composition in dH2O:

| | Concentration of compound |
|---|---|
| Compound | in medium - [g/L] |
| Ampicillin | 0.10 |
| Zeocin | 0.040 |
| Kanamycin | 0.05 |

### Strains and Plasmids

Strains of Escherichia coli used herein were the following:

| | | | CRP-cAMP |
|---|---|---|---|
| | | | relevant |
| Strain name | Plasmid | Relevant description | genotype |
| BS1011 | — | ΔbioB derivative of BS1013¹ | WT |
| BS1013 | — | E. coli K-12 BW25113¹ parent strain: rrnB3 ΔlacZ4787 | WT |
| | | hsdR514 Δ(araBAD)567 Δ(rhaBAD)568 rph-1 | |
| BS1093 | pBS451 | Zeocin resistant and biotin auxotrophic strain used for | WT |
| | | biotin bioassay | |
| BS1353 | — | BS1011 derivative comprising a H107Y mutation in IscR² | WT |
| BS1575 | — | BS1353 derivative with deletion of bioAFCDH | WT |
| BS2720 | | Derivative of BS1575 with frameshift mutation in CyaA | CRP-cAMP |
| | | | defective |
| BS2003 | | BS1011 derivative with deletion of bioAFCDH and | WT |
| | | comprising a H107Y mutation in IscR² | |
| BS4755 | | Derivative of BS1575 with complete deletion of CyaA | CRP-cAMP |
| | | | defective |
| BS4090 | | Derivative of BS1575 with a translation knockout of | CRP-cAMP |
| | | CyaA (MAGE). | defective |
| BS4260 | | Derivative of BS1575 with a translation knockout of CRP | CRP-cAMP |
| | | (MAGE). | defective |
| BS4261 | | Derivative of BS1575 with a translation knockout of CRR | CRP-cAMP |
| | | (MAGE) | defective |
| BS2658 | pBS679 | BS1353 derivative with deletion of bioAFCDH | WT |
| | | comprising a plasmid with IPTG inducible expression of | |
| | | Escherichia coli BioB | |
| BS4118 | pBS679 | Derivative of BS4090 (translational knockout of CyaA) | CRP-cAMP |
| | | comprising a plasmid with IPTG inducible expression of | defective |
| | | Escherichia coli BioB | |
| BS4377 | pBS679 | Derivative of BS4260 (translation knockout of CRP) | CRP-cAMP |
| | | comprising a plasmid with IPTG inducible expression of | defective |
| | | Escherichia coli BioB | |
| BS4378 | pBS679 | Derivative of BS4261 (translation knockout of CRR) | CRP-cAMP |
| | | comprising a plasmid with IPTG inducible expression of | defective |
| | | Escherichia coli BioB | |
| BS3304 | pBS679 | Derivative of BS1575 comprising a plasmid with IPTG | WT |
| | | inducible expression of Escherichia coli BioB | |
| BS4759 | pBS679, | Derivative of BS4755 comprising a plasmid with IPTG | CRP-cAMP |
| | pBS1565 | inducible expression of Escherichia coli BioB and a | defective |
| | | plasmid with constitutive expression of DTB production | |
| | | pathway | |
| BS2154 | pBS679 | Derivative of BS2003 comprising a plasmid with IPTG | WT |
| | | inducible expression of Escherichia coli BioB | |
| BS3079 | pBS679 | Derivative of BS2720 comprising a plasmid with IPTG | CRP-cAMP |
| | | inducible expression of Escherichia coli BioB | defective |
| BS4118 | pBS679 | Derivative of BS4090 with a translation knockout of | CRP-cAMP |
| | | CyaA (MAGE) comprising a plasmid with IPTG inducible | defective |
| | | expression of Escherichia coli BioB | |
| BS6275 | | Derivative of BS1575 with increased expression CpdA | CRP-cAMP |
| | | | defective |
| BS6276 | | Derivative of BS1575 with overexpression of | CRP-cAMP |
| | | heterologous CadD. | defective |

¹Commercially available strain e.g. from The Coli Genetic Stock Center; http://cgsc2.biology.yale.edu/KeioList.php
²WO2019012058A1

Further strains of Escherichia coli used herein were the following:

| | | | | cAMP-CRP |
|---|---|---|---|---|
| | | | IscR | relevant |
| Strain name | Plasmid | Relevant description | genotype | genotype |
| BS00734 | — | ΔthiP derivative of BS01013¹ | WT | WT |
| BS01013 | — | E. coli K-12 BW25113¹ parent strain: rrnB3 ΔlacZ4787 | WT | WT |
| | | hsdR514 Δ(araBAD)567 Δ(rhaBAD)568 rph-1 | | |
| BS04501 | pBS0451 | Zeocin resistant and thiamine auxotrophic strain with a | WT | WT |
| | | KO in ThiC used for a thiamine bioassay | | |
| BS04565 | — | BS00734 derivative with chromosomally integrated | WT | WT |
| | | phosphatase from Arabidopsis thaliana At5g32470² | | |
| BS04701 | — | BS04565 derivative comprising a H107Y mutation in iscR³ | Mutant | WT |
| BS04705 | — | Derivative of BS04701 with a translation knockout of CRP | Mutant | cAMP-CRP |
| | | (MAGE strain). | | defective |
| BS04782 | — | Derivative of BS04701 with complete deletion of CyaA | Mutant | cAMP-CRP |
| | | (recombineering). | | defective |
| BS04608 | pBS2180 | Derivative of BS04565 comprising a plasmid with IPTG | WT | WT |
| | pBS2184 | inducible expression of E. coli ThiC and a plasmid with | | |
| | | constitutive expression of ThiMDE genes from E. coli | | |
| BS04726 | pBS2180 | Derivative of BS04701 comprising a plasmid with IPTG | Mutant | WT |
| | pBS2184 | inducible expression of E. coli ThiC and a plasmid with | | |
| | | constitutive expression of ThiMDE genes from E. coli | | |
| BS04739 | pBS2180 | Derivative of BS04705 comprising a plasmid with IPTG | Mutant | cAMP-CRP |
| | pBS2184 | inducible expression of E. coli ThiC and a plasmid with | | defective |
| | | constitutive expression of ThiMDE genes from E. coli | | |
| BS04786 | pBS2180 | Derivative of BS04782 comprising a plasmid with IPTG | Mutant | cAMP-CRP |
| | pBS2184 | inducible expression of E. coli ThiC and a plasmid with | | defective |
| | | constitutive expression of ThiMDE genes from E. coli | | |

¹Commercially available strain, e.g., from the Coli Genetic Stock Center; http://cgsc2.biology.yale.edu/KeioList.php
²WO2017103221A1
³WO2019012058A1

Plasmids used herein were as follows:

| | | | Antibiotic | DNA |
|---|---|---|---|---|
| Plasmid ID | IPTG inducible expression of | Origin | marker | sequence ID |
| pBS451 | | p15A | Zeo^{R} | |
| pBS679 | BioB [SEQ. ID No. 38] | SC101 | Amp^{R} | SEQ ID NO: |
| | overexpression plasmid from a | | | 38 and 50 |
| | T5LacO repressed promoter | | | |
| | [SEQ ID No. 50] | | | |
| | | | Antibiotic | DNA |
| Plasmid ID | Constitutive expression of | Origin | marker | sequence ID |
| pBS1565 | DTB pathway genes BioF, BioA, | p15A | Kan^{R} | [SEQ. ID No. |
| | BioD, BioC, BioH [SEQ. ID | | | 6, 8, 12, 2, 4] |
| | No. 6, 8, 12, 2, 4, respectively] | | | |

Further plasmids used herein were as follows:

| | | | Antibiotic | DNA |
|---|---|---|---|---|
| Plasmid ID | IPTG inducible expression of | Origin | marker | sequence ID |
| pBS2180 | ThiC [SEQ ID No. 52] | SC101 | Amp^{R} | SEQ ID NO: |
| | overexpression plasmid | | | 52 and 107 |
| | from a T5LacO repressed | | | |
| | promoter [SEQ ID No. 107] | | | |
| | | | Antibiotic | DNA |
| Plasmid ID | Constitutive expression of | Origin | marker | sequence ID |
| pBS451 | Green Fluorescent | p15A | Zeo^{R} | SEQ ID NO: |
| | Protein from Aequorea | | | |
| | victoria | | | |
| pBS2184 | Thiamine pathway genes | pBR322 | Kan^{R} | [SEQ ID No. |
| | ThiM, ThiD, ThiE [SEQ ID | | | 64, 54, 72, 108] |
| | No. 64, 54, 72, | | | |
| | respectively] under | | | |
| | constitutive apFAB71 | | | |
| | promoter (SEQ. ID No. 108) | | | |

### Analytical Procedures

### Procedure I: Optical Densities Measurements

To measure optical densities (OD) of a cell culture as cuvette OD at 600 nm (cOD₆₀₀), the culture was diluted 10-fold with dH₂O to a final volume of 1 mL and transferred to a 1.5 mL transparent cuvette with 10 mm pathlength. The diluted culture was measured at 600 nm and 10 mm pathlength on a mySPEC (VWR). If the diluted culture was measured to cOD₆₀₀>0.4, the culture was further diluted 10-fold and remeasured.

### Procedure II: Bioassay for Vitamin Quantification

For quantification of biotin in supernatant samples from small-scale screening and fermentations, a bioassay involving biotin auxotrophic strain BS1093, mMOPS (excluding biotin) supplemented with zeocin and >5 of biotin standards in the dynamic growth range of BS1093 was used following the assay described in section 1.6 of the methods in the examples in WO2019012058. Specifically, the supernatant from each culture was diluted alongside >5 biotin standards in the concentration range of 0 μM (mg/L) to 40 μM (12.9 mg biotin/L) prepared in Milli-Q water. 15 μl of each diluted supernatant and each of the biotin standards was then added to a well of a 96-well microtiter plate. Each well contained prior to addition 135 μl of mMOPS medium without biotin but with supplementation of zeocin and inoculated to an initial OD₆₀₀ of 0.01 with a biotin-starved overnight culture of BS1093. The plate was sealed with a breathable seal and incubated at 37° C. with 275 rpm shaking for 20 h before OD₆₀₀ was measured. A biotin bioassay calibration curve was obtained using the dynamic range of growth response of BS1093 to the biotin standards. For sample wells, the calibration curve was used to calculate concentrations of biotin produced based on the growth response of BS1093 in the individual wells.

For quantification of thiamine in supernatant samples from small-scale cultivations and fermentations, a bioassay involving thiamine auxotrophic strain BS04501 cultivated in mMOPS supplemented with zeocin and >5 thiamine standards in the dynamic growth range of BS04501 was used following the assay described in section 1.6 of the methods in the examples in WO2019012058. Specifically, the supernatant from each culture was diluted alongside >5 thiamine standards in the concentration range of 0 μM (mg/L) to 60 μM (15.9 mg thiamine/L) prepared in Milli-Q water. 15 μL of each diluted supernatant and each of the biotin standards was then added to a well of a 96-well microtiter plate. Each well contained prior to addition 135 μL of mMOPS medium without thiamine but with supplementation of zeocin and inoculated to an initial OD₆₀₀ of 0.01 with a thiamine-starved overnight culture of BS04501. The plate was sealed with a breathable seal and incubated at 37° C. with 275 rpm shaking for 20 hours before OD₆₀₀ was measured. A thiamine bioassay calibration curve was obtained using the dynamic range of growth response of BS04501 to the thiamine standards. For sample wells, the calibration curve was used to calculate concentrations of thiamine produced based on the growth response of BS04501 in the individual wells.

For quantification of pantothenic acid, similar procedures were followed but using other standards and strains are reported elsewhere:

(https://www.sciencedirect.com/science/article/abs/pii/0076687979622188).

### Procedure III: Measurement of Dry Cell Weight (DCW)

For the quantification of DCW in culture samples harvested from fermentations a known volume of culture was added to a centrifuge tube. Prior to addition this centrifuge tube was dried at 80° C. for 24 h and weighed with the weight recorded. The culture was then spun at 4000 g for 20 min at 4° C., the supernatant carefully removed, and the pellet resuspended in Milli-Q water, then spun at 4000 g for 20 min at 4° C. and again the supernatant carefully removed. This washing step was repeated and then the remaining wet cell mass was dried at 80° C. The dried cell mass was regularly weighed until no further weight loss was recorded. The dried centrifuge tube weight was subtracted from the dried cell mass weight and g/L DCW calculated based on the known volume of culture transferred to the centrifuge tube. This procedure was done in duplicate or triplicate for each culture sample with the final DCW g/L representing an average of measurements.

### Procedure IV (a): Ultra-High Performance Liquid Chromatography Coupled to Tandem Mass Spectrometry (UHPLC-MS/MS) for Analyzing Biotin and Metabolites Related to the Metabolic Pathway of Biotin

### Chemicals and Reagents

Commercial standards for the analytes of interest were purchased. Biotin (BTN) was acquired from Sigma Aldrich, biotin sulfoxide (BX), biotin sulfone (BSN), 7-keto-8-aminopelargomic acid (KAPA) and 7,8-diaminopelargonic acid (DAPA) from Santa Cruz Biotechnology and d-desthiobiotin (DTB) from Biosynth Carbosynth. The internal standards d₄-biotin (d₄-BTN) and ¹³C₅-biotin sulfoxide (¹³C₅—BX) were purchased from Sigma Aldrich. Water (H₂O) and acetonitrile (ACN) were purchased from Honeywell and dimethyl sulfoxide (DMSO) and acetic acid (CH₃COOH) were purchased from Carl Roth. Stock solutions of the analytes and internal standards were prepared in DMSO to a concentration of 1 mg mL⁻¹. Working standard solutions of the stock solutions were then prepared in H₂O. Calibration curves in the concentrations of 0.1, 0.25, 0.5, 1, 2.5, 5, 10, 25, 50, 100, 250, 500 and 1000 ng mL⁻¹ were prepared in H2O containing 50 ng mL⁻¹ of ¹³C₅—BX and 5 ng mL⁻¹ of d₄-BTN. An internal standard mixture (ISTD MIX) containing 5 μg mL⁻¹ of ¹³C₅—BX and 0.5 μg mL⁻¹ of d₄-BTN was prepared in H₂O.

### Sample Preparation

Before analyses, the samples from the bioreactors are diluted and a mixture of internal standards (ISTD MIX) is added to correct for possible technical variation. The dilution factor differs depending on which of the analytes are to be quantified. For the quantification of BX, BSN, KAPA and DAPA a 1:10 dilution is prepared. First 890 μL of H₂O is pipetted into a glass vial. Then 10 μL of the ISTD MIX and 100 μL of the original sample are added, and the solution is vortex mixed. For the quantification of BTN and DTB a two-step dilution pattern is followed to achieve a 1:1000 dilution of the original sample. First a 1:10 dilution is created by pipetting 900 μL of H₂O and 100 μL of the original sample into a glass vial. The solution is vortex mixed. Finally, 980 μL of H₂O, 10 μL of the ISTD MIX and 10 μL of the 1:10 diluted sample are pipetted into a vial and the solution is vortex mixed.

### Liquid Chromatography-Tandem Mass Spectrometry

The samples are randomized after sample preparation and analyzed by ultra-high performance liquid chromatography (Infinity II, Agilent Technologies) coupled to tandem mass spectrometry (6470 Triple Quadrupole, Agilent Technologies) using electrospray ionization in positive ion mode. Selected reaction monitoring is used for quantifying the analytes. Fragmentor voltages, collision energies and cell accelerator voltages are optimized for each ion transition.

The analytes are separated chromatographically before they enter the mass spectrometer. This is done using a ACQUITY UPLC HSS T3 Column (2.1 mm×100 mm, particle size 1.8 μm, Waters Corporation) and H₂O+0.1% (v/v) CH₃COOH as eluent A and ACN+0.1% (v/v) CH₃COOH as eluent B with a flow rate of 0.4 mL min⁻¹. The elution gradient is as follows: 0-0.5 min 0% B, 0.5-1.5 min 0% to 15% B, 1.5-3 min 15% B, 3-5 min 15% to 100% B, 5-7 min 100% B. After each run, the column is re-equilibrated at 0% B for 2 min. The injection volume for each sample is 5 μL. All data is acquired using the MassHunter Acquisition software (Version 10.0, Build 10.0.142).

### Data Processing

All data is processed using the MassHunter Quantitative Analysis software (Version B.09.00, Build 9.0.647.0). The peak areas for the analytes of interest are normalized against the peak areas of the corresponding internal standards (BSN, BX, DAPA and KAPA are normalized against ¹³C₅—BX and BTN and DTB are normalized against d₄-BTN). Data quality is ensured by evaluating technical replicates of a specific fermentation sample as well as commercial standards. The quantified concentrations of BTN and DTB in these samples are required to remain within ±10% for the data set to be approved.

### Molecular Biology Techniques

Standard techniques were used for DNA isolation, amplification, purification and cloning (restriction digestion, ligation), transformation and the like. Such techniques are well known in the art and standard protocols can be found in: Maniatis et al., 1982 Molecular Cloning, Cold Spring Harbor Laboratory, Plainview NY. And M. Green, J. Sambrook (2012) Molecular Cloning: a laboratory manual. 4^{th} Edition, Cold Spring Harbor Laboratory Press, CSH, NY., which are both hereby incorporated by reference in their entireties.

### Procedure IV (b): Ultra-High Performance Liquid Chromatography Coupled to Tandem Mass Spectrometry (UHPLC-MS/MS) for Analyzing Thiamine and Metabolites Related to the Metabolic Pathway of Thiamine

### Chemicals and Reagents

Commercial standards for the analytes of interest were purchased. thiamine was acquired from Sigma Aldrich, thiamine sulfoxide (BX), thiamine sulfone (BSN), 7-keto-8-aminopelargomic acid (KAPA) and 7,8-diaminopelargonic acid (DAPA) from Santa Cruz Biotechnology and d-desthiothiamine (DTB) from Biosynth Carbosynth. The internal standards d₄-thiamine (d₄-BTN) and ¹³C₅-thiamine sulfoxide (¹³C₅—BX) were purchased from Sigma Aldrich. Water (H₂O) and acetonitrile (ACN) were purchased from Honeywell and dimethyl sulfoxide (DMSO) and acetic acid (CH₃COOH) were purchased from Carl Roth.

Stock solutions of the analytes and internal standards were prepared in DMSO to a concentration of 1 mg/mL. Working standard solutions of the stock solutions were then prepared in H₂O. Calibration curves in the concentrations of 0.1, 0.25, 0.5, 1, 2.5, 5, 10, 25, 50, 100, 250, 500 and 1000 ng/ml were prepared in H2O containing 50 ng/ml of ¹³C₅—BX and 5 ng/ml of d₄-BTN. An internal standard mixture (ISTD MIX) containing 5 μg/mL of ¹³C₅—BX and 0.5 μg/mL of d₄-BTN was prepared in H₂O.

### Sample Preparation

Before analyses, the samples from the bioreactors were diluted and a mixture of internal standards (ISTD MIX) is added to correct for possible technical variation. The dilution factor differs depending on which of the analytes were to be quantified. For the quantification of BX, BSN, KAPA and DAPA a 1:10 dilution is prepared. First 890 μL of H₂O is pipetted into a glass vial. Then 10 μL of the ISTD MIX and 100 μL of the original sample were added, and the solution was vortex mixed. For the quantification of BTN and DTB a two-step dilution pattern was followed to achieve a 1:1000 dilution of the original sample. First a 1:10 dilution was created by pipetting 900 μL of H₂O and 100 μL of the original sample into a glass vial. The solution was vortex mixed. Finally, 980 μL of H₂O, 10 μL of the ISTD MIX and 10 μl of the 1:10 diluted sample were pipetted into a vial and the solution is vortex mixed.

### Liquid Chromatography-Tandem Mass Spectrometry

The samples were randomized after sample preparation and analyzed by ultra-high performance liquid chromatography (Infinity II, Agilent Technologies) coupled to tandem mass spectrometry (6470 Triple Quadrupole, Agilent Technologies) using electrospray ionization in positive ion mode. Selected reaction monitoring was used for quantifying the analytes. Fragmentor voltages, collision energies and cell accelerator voltages were optimized for each ion transition.

The analytes were separated chromatographically before they enter the mass spectrometer. This was done using a ACQUITY UPLC HSS T3 Column (2.1 mm×100 mm, particle size 1.8 μm, Waters Corporation) and H₂O+0.1% (v/v) CH₃COOH as eluent A and ACN+0.1% (v/v) CH₃COOH as eluent B with a flow rate of 0.4 mL min⁻¹. The elution gradient was as follows: 0-0.5 min 0% B, 0.5-1.5 min 0% to 15% B, 1.5-3 min 15% B, 3-5 min 15% to 100% B, 5-7 min 100% B. After each run, the column was re-equilibrated at 0% B for 2 min. The injection volume for each sample was 5 μL. All data is acquired using the MassHunter Acquisition software (Version 10.0, Build 10.0.142).

### Data Processing

All data was processed using the MassHunter Quantitative Analysis software (Version B.09.00, Build 9.0.647.0). The peak areas for the analytes of interest were normalized against the peak areas of the corresponding internal standards (BSN, BX, DAPA and KAPA were normalized against ¹³C₅—BX and BTN and DTB were normalized against d₄-BTN). Data quality was ensured by evaluating technical replicates of a specific fermentation sample as well as commercial standards. The quantified concentrations of BTN and DTB in these samples were required to remain within +10% for the data set to be approved.

### Molecular Biology Techniques

Standard techniques were used for DNA isolation, amplification, purification, and cloning (restriction digestion, ligation), transformation and the like. Such techniques were well known in the art and standard protocols can be found in for example: Maniatis et al., 1982 Molecular Cloning, Cold Spring Harbor Laboratory, Plainview NY. And M. Green, J. Sambrook (2012) Molecular Cloning: a laboratory manual. 4^{th} Edition, Cold Spring Harbor Laboratory Press, CSH, NY., which were both hereby incorporated by reference in their entireties.

### Example 1—Construction of CyaA, CRR and CRP Knockout Strains

CRP is activated as a transcriptional regulator by binding to its allosteric activator cyclic AMP (cAMP) which is produced by the enzyme adenylate cyclase (encoded by CyaA). Under low-glucose conditions adenylate cyclase is activated by the phosphorylated state of glucose-specific enzyme IIA or EIIA (encoded by CRR). Mutations to effectively knock out CyaA, CRR and CRP were introduced into parent such as BS1575 by multiplex automated genome engineering (MAGE) as described in Methods in Enzymology, 498, 409-426, 2011 using the DNA oligos shown in the Table 1 to introduce the desired mutations. Translational knockouts were generated by introducing 3 stop codons immediately following the start codon of the gene to eliminate any translation of the relevant polypeptide and separately a frameshift mutation was introduced into CyaA (at L169 of the translated polypeptide) using oligo moBS506 to eliminate production of a functional protein.

TABLE 1
Oligos used to introduce mutations

| Mutation | | | Resulting |
|---|---|---|---|
| description | Oligo name | Oligo sequence | strain |
| CyaA (L169 | moBS506 | TGGAGCCACAATCTTCGCCCCCAGGCTGCCGCTTTCATTA | BS4088 |
| frameshift) | | TGACGGAAGCGGTTTTCATCAAT (SEQ ID NO: 127) | |
| CyaA translational | moBS508 | AAGCGCGCGATCCACACGCAATTGATTTATGGCATCCAGT | BS4262 |
| KO | | CaCTaTTaCAGAGTCTCAATATAGAGGTACAAGACGTATC | |
| | | GCCTGATTTG (SEQ ID NO: 128) | |
| CRP translational | moBS509 | CTTGGCAAACCGCAAACAGACCCGACTCTCtAATaGTgaTT | BS4260 |
| KO | | GTCTCATTGCCACATTCATAAGTACCCATCCAAGAGCACG | |
| | | CTTATTCAC (SEQ ID NO: 129) | |
| CRR translational | moBS510 | ACGAGATGCGGCCCAATTTACTGCTTAGGAGAAGATCAT | BS4261 |
| KO | | GtGaTaGTaaGATAAACTGAAATCTCTGGTTTCCGACGAC | |
| | | AAGAAGGATAC (SEQ ID NO: 130) | |

Successful introduction of the desired mutations was verified by PCR amplification of the region followed by Sanger sequencing.

An alternative to translational knockouts is the complete removal of a gene from the chromosome by methods known in the art. To construct strain BS4755 the gene CyaA was completely removed from the chromosome of strain BS1575 using methods known in the art and replaced with only a short FRT sequence. One method to achieve this removal of the CyaA gene is by generating a DNA fragment carrying a Kanamycin resistance gene flanked by homologous regions of CyaA and transforming this DNA into strain BS1575 carrying the λRed recombinase genes expressed from an inducible promoter. One protocol for such a method can be found in Datsenko, K. A. and Wanner, B. L., One-step inactivation of chromosomal genes in Escherichia coli K-12 using PCR products, PNAS, 2000, 97 (12): 6640-5, DOI: 10.1073/pnas.120163297.

### Example 2—Construction of Strains Overexpressing cAMP-Degrading Enzymes CpdA and CadD

cAMP phosphodiesterase (CpdA) hydrolyzes cAMP, thereby regulating levels in E. coli. Overexpression of CpdA increases hydrolysis of cAMP and therefore decreases the levels of functional CRP-cAMP complex.

cAMP deaminase (CadD) is an enzyme found in, e.g., Leptospira interrogans that deaminates cAMP to cyclic-3′,5′-inosine monophosphate (ACS Chem. Biol. 2013, 8, 12, 2622-2629). Heterologous expression of CadD in E. coli leads to degradation of cAMP and therefore decreases the levels of functional CRP-cAMP complex.

To construct a strain overexpressing CpdA (BSBS6275) or CadD (BSBS6276) the respective genes are expressed using a constitutive or inducible promoter from a plasmid or from the chromosome. To generate said expression plasmids the genes are amplified using Phusion U polymerase (Thermo Fischer Scientific) following manufacturer's protocol and using primers containing uracil for recognition by USER restriction enzymes. Similarly, plasmid backbones are amplified. DNA fragments are digested and ligated using USER enzyme (New England Biolabs) and T4 ligase (Thermo Fischer Scientific) following the manufacturers' protocols. These mixtures are transformed by electroporation into BS1575, and the transformed cells are grown on selective LB agar overnight at 37° C. To express the genes from the chromosome of E. coli they are introduced as DNA fragments carrying a promoter, the gene, an antibiotic resistance cassette and flanking regions homologous to the integration site into the chromosome using methods known in the art such as by transforming the respective DNA into strain carrying the λRed recombinase genes expressed from an inducible promoter and selecting for antibiotic resistance.

### Example 3—Construction of Strains Carrying Genes for Providing Biotin Production

pBS679: An IPTG-inducible (Promoter SEQ ID NO: 49) transgene encoding BioB (SEQ ID NO: 38) was cloned on plasmid pBS679 by amplification of the gene and a ribosome binding site using Phusion U polymerase (Thermo Fischer Scientific) following manufacturer's protocol and using primers containing uracil for recognition by USER restriction enzymes. Similarly, a plasmid backbone carrying origin pSC101 and an Ampicillin resistance cassette as well as the promoter sequence was amplified. DNA fragments were digested and ligated using USER enzyme (New England Biolabs) and T4 ligase (Thermo Fischer Scientific) following the manufacturers' protocols. These mixtures were transformed by electroporation into BS1575 and transformed cells were grown on selective LB agar supplemented with ampicillin overnight at 37° C.

PBS1565: A plasmid encoding genes BioFADCH (SEQ ID Nos. 6, 8, 12, 2, 4, respectively) driven by constitutive promoter apFAB346 (SEQ ID No: 50) was cloned on a plasmid backbone carrying a Kanamycin resistance cassette and pBR322 origin of replication using USER cloning methods as described above for pBS679 construction. Each gene was preceded by a ribosome binding site.

Transformation: Plasmids pBS679 and/or pBS1565 were transformed into background strains of interest using electroporation transformation protocols well known in the art and transformant strains were selected on selective Agar plates contains Ampicillin (pBS679), Kanamycin (pBS1565) or both.

### Example 4—Growth of CyaA, CRR and CRP Knockout Strains on Glucose Vs. Succinate

E. coli strains require a functional CRP-cAMP complex to express pathways allowing utilization of alternative carbon sources such as succinate. A strain that cannot form a functional CRP-cAMP complex cannot grow on succinate as a sole carbon source. To confirm that CyaA, CRR and CRP knockout strains were all defective in the formation of CRP-cAMP, growth of the CyaA, CRR and CRP knockout strains of example 1 were tested on medium containing glucose or succinate as the sole carbon source.

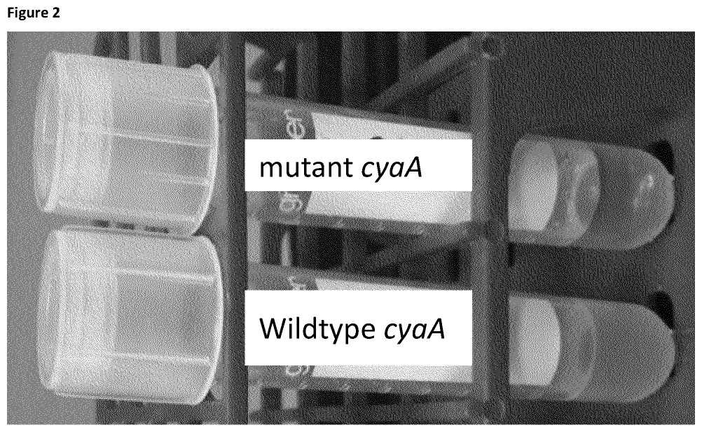

To show visually that strains defective in CyaA are unable to grow on succinate a colony of strain BS1575 (wt) and a colony of strain BS4264 (CyaA mutant) were inoculated in 2 mL of mMOPS medium containing 2 g/L succinate as the sole carbon source. The cultures were incubated at 37° C. for 24 h while shaking. The culture of BS1575 was grown to high density after this time while the culture of BS4264 showed no detectable growth (FIG. 2). Both strains grow equally well in rich medium (not shown).

Similarly strains BS4260, BS4261, BS4262 and control strain BS1575 were inoculated from a single colony each in 200 μL of mMOPS medium containing either 2 g/L succinate or 2 g/L glucose in a microtiter plate and incubated at 37° C. for 24 h while shaking. The resulting cultures were measured for growth by measuring absorbance at 600 nm in a spectrophotometer. As shown in table 2 below all strains grew to high density on glucose medium while only the control strain BS1575 was able to grow on succinate medium.

TABLE 2
experimental observation of growth of different E. coli
strain in liquid medium containing either glucose or succinate
as a sole carbon source. Strains with deactivating mutations
(translational knockouts) in CRP, CRR and CyaA are all unable
to grow on succinate as a sole carbon source. Growth (OD600)
was measured using a plate-reader.

| | | Growth on | Growth on |
|---|---|---|---|
| Strain ID | Relevant genotype | glucose | succinate |
| BS1575 | Wildtype | +++ | +++ |
| BS4260 | CRP translational KO | +++ | − |
| BS4261 | CRR translational KO | +++ | − |
| BS4090 | CyaA translational KO | +++ | − |

### Example 5—Growth of CpdA and CadD Overexpression Strains on Glucose Vs. Succinate

E. coli strains require a functional CRP-cAMP complex to express pathways allowing utilization of alternative carbon sources such as succinate. Strains overexpressing CpdA or CadD (two enzymes that degrade cAMP) have lower levels of functional CRP-cAMP complex and therefore cannot grow on succinate as a sole carbon source. To show that CRP-cAMP complex formation is decreased in strains overexpressing CpdA or CadD their growth is tested using succinate as a sole carbon source.

Strains BS6275, BS6276 and control strain BS1575 are inoculated from a single colony each in 200 μL of mMOPS medium containing either 2 g/L succinate or 2 g/L glucose in a microtiter plate and incubated at 37° C. for 24 h while shaking. The resulting cultures are measured for growth by measuring absorbance at 600 nm in a spectrophotometer. As shown in table 3 all strains grow to high density on glucose medium while only the control strain BS1575 grow on succinate medium.

TABLE 3
growth of different E. coli strain in liquid medium
containing either glucose or succinate as a sole carbon
source. Strains overexpressing CpdA or CadD grow poorly
or not at all on succinate as a sole carbon source.

| | | Growth on | Growth on |
|---|---|---|---|
| Strain ID | Relevant genotype | glucose | succinate |
| BS1575 | Wildtype | +++ | +++ |
| BS6275 | CpdA increased expression | +++ | −/+ |
| BS6276 | CadD overexpression | +++ | −/+ |

### Example 6: BioB Activity is Increased in Strains Defective in Formation of a CRP-cAMP Complex Vs Control

To show that biotin synthases (BioB) activity is greatly increased in E. coli strains defective in CRP-cAMP complex formation such strains overexpressing BioB were cultivated in the presence of biotin precursor desthiobiotin (DTB) and the production of biotin in each culture was measured compared to a control culture.

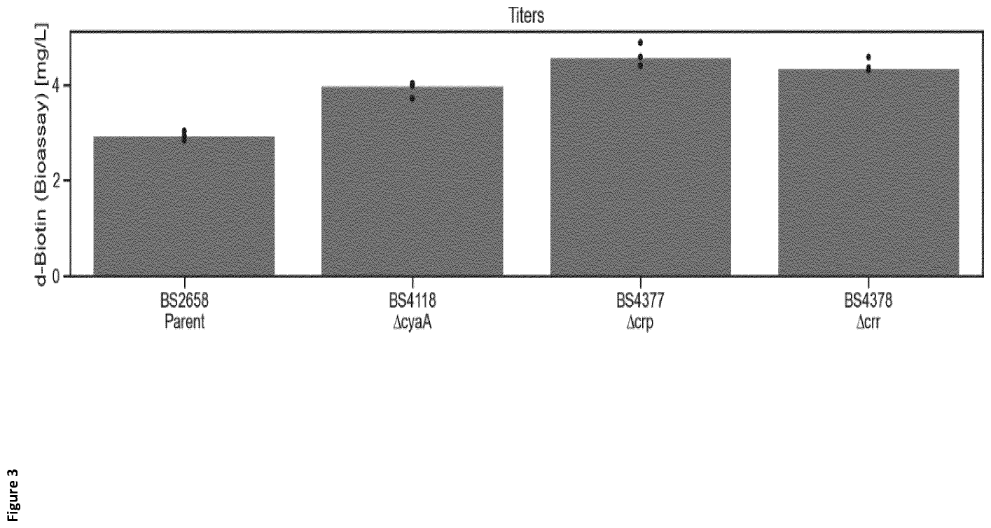

The strains tested all expressed BioB from a plasmid. Control parent strain (BS2658) and KO mutants of CRR, CyaA and CRP (BS4378, BS4118 and BS4377, respectively) are described above in the section: Strains and plasmids. BioB expression was IPTG inducible and the IPTG concentration that gives maximal activity was chosen for each strain. All strains were cultured, in triplicate, in 400 μL mMOPS medium containing 100 μg/mL ampicillin under optimal IPTG concentrations for induction of BioB and in the presence of 0.1 g/L biotin synthase substrate (DTB). The strains were grown in deep well plates for 24 h at 37° C. shaken at 275 rpm, after which biotin production was evaluated using a growth-based bioassay according to procedure II. The resulting biotin titers are shown in FIG. 3. Bars illustrate the median biotin production value (height), black dots show biotin production from individual replicate cultures. The data shows significantly higher biotin titers in all three strains defective in CRP-cAMP formation (CRR, CyaA and CRP knockout strains) than the control strain.

### Example 7: Increase in BioB Activity in Strains Defective in CRP-cAMP Complex Formation Exceeds Increase in Biomass

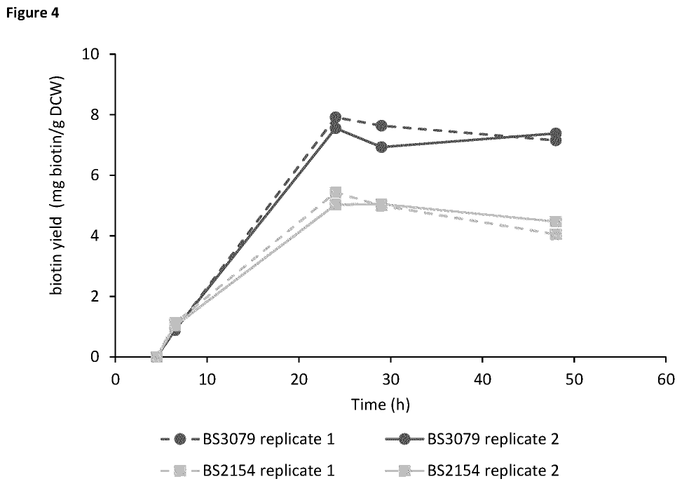

It has been reported that strains defective in CRP-cAMP formation increase the yield of cell biomass produced from glucose (Perrenoud & Sauer (2005)). To test if the biotin titer increases seen in example 6 were simply due to increases in biomass formation per gram of glucose both biotin titers and biomass levels were carefully measured in a fed-batch cultivation of two strains expressing BioB with added DTB: a wildtype strain (BS2154) and a strain with a frameshift mutation in CyaA (BS3079). A 1 mL glycerol stock of BS3079 (BioB overexpressing strain with a CyaA knockout genotype) and BS2154 (BioB overexpressing strain with a CyaA positive genotype) were inoculated into two separate 250 ml shake flasks containing 50 mL mMOPS medium with ampicillin. The shake flasks were incubated at 37° C. and 250 rpm shake for 17 h resulting in an optical density of cOD₆₀₀=2.59 and COD₆₀₀=2.62 respectively. 200 mL B medium supplemented with ampicillin, 0.4 g/L desthiobiotin and 1 ml of a 1% (v/v) antifoam solution was added to four Applikon 500 mL MiniBio Reactors with temperature set to 37° C., pH controlled to pH=7 by addition of 5 M NH₄OH and dissolved oxygen (DO) set-point to DO=15% by agitation speed, sparging was fixed at 1 VVM. 10 ml of each strain preculture was used to inoculate the B medium in duplicate reactors resulting in an initial cOD₆₀₀=0.13 for all reactors and strains. Once the CO₂ in the outlet gas reached greater than 0.4% each fermentation was induced by addition of isopropyl β-D-1-thiogalactopyranoside (IPTG) to a concentration of 0.0191 g/L for BS3079 (4.5 h) and 0.006 g/L for BS2154 (3.8 h), the concentrations represented previously determined induction optimums for biotin production. Following the depletion of glucose in the medium a fed batch phase was initiated by the addition of F medium to each fermentation at a constant feed rate of 1.6 ml/h. This transition took place between 8.5-11 h. The DO control continued to operate at DO=15% with the agitation increasing in all reactors to between 1250-1800 rpm. Additional 1 mL 1% (v/v) antifoam solution was added at 7.00 h and additional 0.4 g/L desthiobiotin was added at 24 h. The fermentations were terminated at 48 h. Culture samples were taken from the fermenter at, 4.5, 6.5, 24, 29 and 48 h after inoculation. From these samples optical density (cOD₆₀₀) was measured according to analytical procedure I and DCW (g/L) measured according to analytical procedure III. Supernatants were obtained by spinning biomass down in a microcentrifuge at 17,000 G for 1 min. Supernatants were screened for biotin using LC-MS method described in analytical procedure IV. From the data the biotin yield per g DCW was calculated (mg biotin produced/g DCW produced) this calculation can be seen over the course of the fermentation in FIG. 4 and shows that by 48 h the mg biotin produced per g DCW was approximately 2-fold greater in the strain without a functional CRP-cAMP complex (BS3079) compared to the control strain with a functional CRP-cAMP complex (BS2154).

This clearly showed that the increase in biomass does not explain the increased BioB activity. Increase in biotin production by mutating CyaA is much higher than increase in biomass. The CyaA frameshift mutation and thereby the elimination of CRP-cAMP complex in the cell indeed increases the activity of BioB per cell.

### Example 8: De Novo Biotin Production in Fed Batch Fermentation of a Strain Defective in Formation of a Functional CRP-cAMP Complex Compared to a Strain with a Functional CRP-cAMP Complex

To determine if mutations in CRP-cAMP complex formation improve biotin production directly from glucose (de novo) biotin production was measured in fed-batch fermentation in representative CyaA wildtype and knockout strains each carrying two plasmids: pBS1565 (DTB production genes) and pBS679 (BioB).

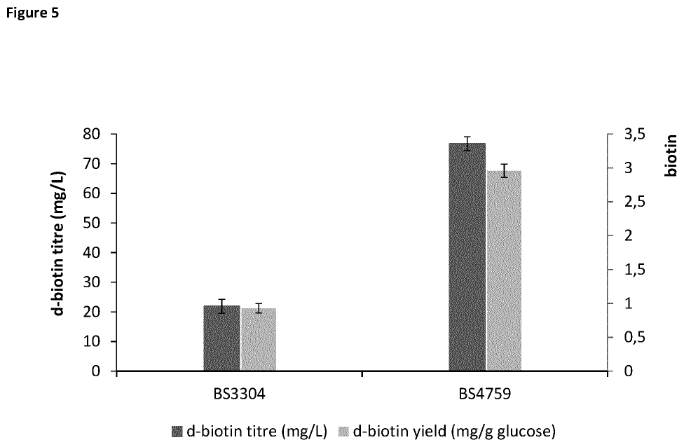

A 1 ml glycerol stock of BS4759 (de novo biotin producing strain with a CyaA knockout genotype) and BS3304 (de novo biotin producing strain with a CyaA positive genotype) were inoculated into two separate 250 mL shake flasks containing 50 mL mMOPS medium with ampicillin and kanamycin. The shake flasks were incubated at 37° C. and 250 rpm shake for 20 h resulting in an optical density of COD₆₀₀ 2.5-3.5. 0.9 mL B medium supplemented with ampicillin and kanamycin was added to a BioLector 32 well microfluidic plate equipped with pH, DO and biomass fluorescence (m2p-labs). 5% (v/v) of each strain culture was used to inoculate the B medium in triplicate fermentation wells resulting in an initial cOD₆₀₀ 0.125-0.175 for each well and strain. The microfluidic plate was then sealed with gas-permeable sealing foil with evaporation reduction and loaded into the BioLector Pro (m2p-labs, Baesweiler, Germany). The temperature set to 37° C., pH controlled in each well to pH=7 by addition of 5 M NH₄OH, the agitation speed of the plate set to 1300 rpm (3 mm orbit) and the relative humidity in the chamber controlled at 85%. Four hours after inoculation the plate fermentations were paused, and each culture well was induced by addition of isopropyl β-D-1-thiogalactopyranoside (IPTG) to a concentration of 0.0048 g/L. The fermentation was resumed and following the depletion of glucose in the batch medium a fed batch phase was initiated by the addition of G medium to each culture well. This addition was controlled to an unlimited 1 μL pulse provided the DO % in each well registered greater than 70%. This transition took place between 6.7-11 h. The fermentations were terminated at 24 h. An endpoint culture sample was taken from each fermentation well at 24 h after inoculation. From this sample optical density (cOD₆₀₀) was measured according to analytical procedure I. Supernatants were obtained by spinning biomass down in a centrifuge at 4000 g, 5 min at 4° C. Supernatants were screened for biotin using LC-MS method described in analytical procedure IV. The final biotin yield was calculated by normalizing the mg biotin produced by the total g glucose fed to each well. The biotin titre and yield for each strain can be seen in FIG. 5 and shows that the de novo biotin titre and yield were approximately 3-fold greater in the strain without a functional CRP-cAMP complex (BS4759) compared to the control strain with a functional CRP-cAMP complex (BS3304).

### Example 9: BioB Activity is Increased in CpdA and CadD Overexpression Strains Compared to Control

Strains overexpressing CpdA and CadD (BS6275 and BS6276) as described in Example 2 are transformed with pBS679 plasmid carrying BioB. Strain BS2658 is used as a control. BioB expression is IPTG inducible and the IPTG concentration that gives maximal activity is chosen for each strain. All strains are cultured, in triplicate, in 400 μL mMOPS medium containing 100 μg/mL ampicillin under optimal IPTG concentrations for induction of BioB and in the presence of 0.1 g/L biotin synthase substrate (DTB). The strains are grown in deep well plates for 24 h at 37° C. shaken at 275 rpm, after which biotin production is evaluated using a growth-based bioassay according to procedure II. The results in table 4 below show significantly higher biotin titers in the strains overexpressing CpdA and CadD than the control strain indicating that overexpression of cAMP degrading enzymes also improves biotin production.

TABLE 4
Comparison of biotin production of three strains.

| | Known effect on | Effect on biotin |
|---|---|---|
| Genetic mutation | CRP-cAMP complex | production |
| Wildtype | wildtype | wildtype |
| Increased expression of cAMP | decrease | Increased |
| phosphodiesterase CpdA | | |
| Increased expression of cAMP | decrease | Increased |
| deaminase CadD | | |

### Example 10—Construction of Further IscR, CRP and CyaA Mutant Strains

Using the ΔthiP mutant BS00734 as starting strain, a PCR cassette of the Arabidopsis thaliana phosphatase was introduced into the chromosome at location KO-176 via the “clonetegration” method generally known in the art. The resulting strain BS04565 was used as the starting point for mutagenesis of IscR. This transcription factor regulates the expression of dozens of genes involved in the biosynthesis of FeS clusters. By mutating IscR to favor the apoprotein formation, with the aim to improve thiamine production. Accordingly, IscR was mutated in BS04565 by multiplex automated genome engineering (MAGE) as described in Methods in Enzymology, 498, 409-426, 2011 using the DNA oligo shown in Table 5, yielding strain BS04701.

CRP was activated as a transcriptional regulator by binding to its allosteric activator cyclic AMP (cAMP) which was produced by the enzyme adenylate cyclase (encoded by cyaA). Under low-glucose conditions adenylate cyclase was activated by the phosphorylated state of glucose-specific enzyme IIA or EIIA (encoded by CRR). Mutations to effectively knock out CRP were introduced into parent strain BS04701 by MAGE. Translational knockouts were generated by introducing 3 stop codons immediately following the start codon of the gene to eliminate any translation of the relevant polypeptide with the DNA oligo shown in Table 5. An alternative to translational knockouts was the complete removal of a gene from the chromosome by methods known in the art. To construct strain BS04782, the gene cyaA was completely removed from the chromosome of strain BS04701 using methods known in the art and replaced with only a short FRT sequence. One method to achieve this removal of the cyaA gene was by generating a DNA fragment carrying a Kanamycin resistance gene flanked by homologous regions of cyaA and transforming strain BS04701 (carrying the λRed recombinase genes expressed from an inducible promoter) with this DNA cassette. One protocol for such a method can be found in Datsenko, K. A. and Wanner, B. L., One-step inactivation of chromosomal genes in Escherichia coli K-12 using PCR products, PNAS, 2000, 97 (12): 6640-5, DOI: 10.1073/pnas.120163297.

TABLE 5
Oligos used to introduce mutations

| Mutation | | | Resulting |
|---|---|---|---|
| description | Oligo name | Oligo sequence | strain |
| IscR mutation | moBS151 | TCGCCTAAAGTAATGTTGTTGAGAAAACCGGTGAG | BS04701 |
| H107Y | (MAGE) | ACGGTCGCTCAAATCACGCCACAGCGCGTaGGTCAG | |
| | | GCATTTATCGCCGCCCTGG (SEQ ID NO: 117) | |
| IsCR FW | oBS3225 | AATGCTTGACGTTGCGCTCAA (SEQ ID NO: 118) | — |
| | (Check) | | |
| IsCR RV | oBS3226 | GTGGTTGCGGAGTAGTCGAGAT (SEQ ID NO: 119) | — |
| | (Check) | | |
| CRP | moBS509 | CTTGGCAAACCGCAAACAGACCCGACTCTCtAATaG | BS04705 |
| translational KO | (MAGE) | TgaTTGTCTCATTGCCACATTCATAAGTACCCATCCA | |
| | | AGAGCACGCTTATTCAC (SEQ ID NO: 120) | |
| CRP FW | oBS3033 | GGCGTTATCTGGCTCTGGAGAA (SEQ ID NO: 121) | — |
| | (Check) | | |
| CRP RV | oBS3034 | ACGAGAACAGCCGACAATCTGA (SEQ ID NO: 122) | — |
| | (Check) | | |
| CyaA deletion | oBS4519 | GTTGGCGGAATCACAGTCATGACGGGTAGCAAATC | BS04782 |
| FW | (KO) | AGGCGATACGTCTTGGCATTACACGTCTTGAGCGAT | |
| | | TGTG (SEQ ID NO: 123) | |
| CyaA deletion | oBS4520 | CGGATAAGCCTCGCTTTCCGGCACGTTCATCACGAA | BS04782 |
| RV | (KO) | AAATATTGCTGTAATGGGAATTAGCCATGGTCCATA | |
| | | TGAAT (SEQ ID NO: 124) | |
| CyaA FW | oBS4046 | CGAAATATCCCGAATGCCGCGT (SEQ ID NO: 125) | — |
| | (Check) | | |
| CyaA RV | oBS4047 | CGTCGCACGGCACCATATTCCA (SEQ ID NO: 126) | — |
| | (Check) | | |

Successful introduction of the desired mutations was verified by PCR amplification of the region followed by Sanger sequencing using the oligos in Table 5.

### Example 11—Construction of Strains Carrying Genes for Providing Thiamine Production

pBS2180: An IPTG-inducible (Promoter SEQ ID NO: 107) transgene encoding ThiC (SEQ ID NO: 52) was cloned on plasmid pBS2180 by amplification of the gene from E. coli 's chromosome and a ribosome binding site using Phusion U polymerase (Thermo Fischer Scientific) following manufacturer's protocol and using primers containing Uracils for recognition by USER restriction enzymesly, a plasmid backbone carrying origin pSC101 and an Ampicillin resistance cassette as well as the promoter sequence was amplified. DNA fragments were digested and ligated using USER enzyme (New England Biolabs) and T4 ligase (Thermo Fischer Scientific) following the manufacturers' protocols. These mixtures were introduced by electroporation into BS04608 and transformed cells were grown on selective LB agar supplemented with ampicillin overnight at 37° C.

pBS2184: A plasmid encoding genes ThiMDE (SEQ ID Nos. 64, 4, 72, respectively) driven by constitutive promoter apFAB71 (SEQ ID No: 108) was cloned on a plasmid backbone carrying a Kanamycin resistance cassette and pBR322 origin of replication using USER cloning methods as described above for pBS2180 construction. Each gene, except ThiD that is located downstream of ThiM, was preceded by a ribosome binding site.

Transformation: Plasmids pBS2180 and/or pBS2184 were introduced into background strains of interest using electroporation transformation protocols that were well known in the art and transformant strains were selected on selective agar plates contains ampicillin (pBS2180), kanamycin (pBS2184) or both.

### Example 12—ThiC Activity is Increased in Strains Defective in Formation of a CRP-cAMP Complex Compared to Controls

To show that phosphomethylpyrimidine synthase (ThiC) activity is greatly increased in E. coli strains defective in CRP-cAMP complex formation such strains overexpressing key thiamine genes (ThiMDE) were cultivated in mMOPS with thiazole supplementation (THZ) and the production of thiamine in each culture was measured compared to a control culture.

The strains tested all expressed ThiC from a first plasmid (pBS2180) and ThiMDE (pBS2184) from a second plasmid. Control parent strains (BS04608) and IscR mutant (BS04726) as well as KO mutants of CRP (BS04739) and CyaA (BS04786) were described above in the section: Strains and plasmids. ThiC expression was IPTG inducible and the IPTG concentration that gave maximal activity was chosen for each strain, whereas ThiMDE expression was constitutive. All strains were cultured, in 4 replicates in 400 μL mMOPS medium containing 100 μg/mL ampicillin under optimal IPTG concentrations for induction of ThiC and 50 μg/mL kanamycin for plasmid expressing ThiMDE.

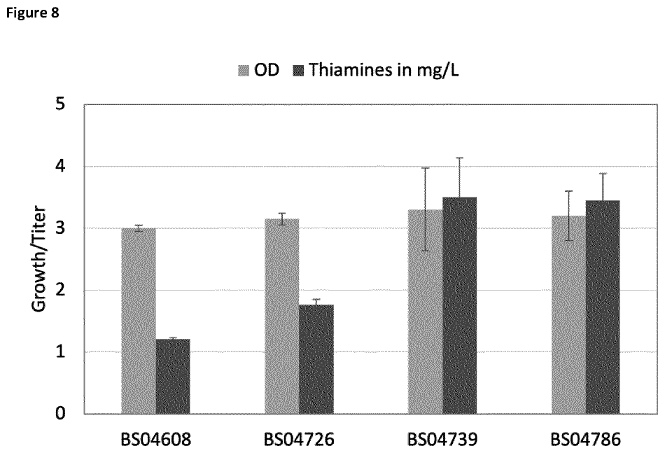

The strains were grown in deep well plates for 24 hours at 37° C. shaken at 275 rpm, after which thiamine production was evaluated using a growth-based bioassay according to procedure II. The resulting thiamine titers were shown in FIG. 8. Bars illustrate the median thiamine production value (height), black dots show thiamine production from individual replicate cultures. The data shows significantly higher biotin titers in both strains defective in CRP-cAMP formation (CRP and CyaA knockout strains) than the control strains (IscR WT or mutant genotype).

### Example 13—Thiamine Production in Fed Batch Fermentation of a Strain Defective in Formation of a Functional CRP-cAMP Complex Compared to Strains with a Functional CRP-cAMP Complex

To determine if mutations in CRP-cAMP complex formation improve thiamine production, fed-batch fermentations were performed using all strains.

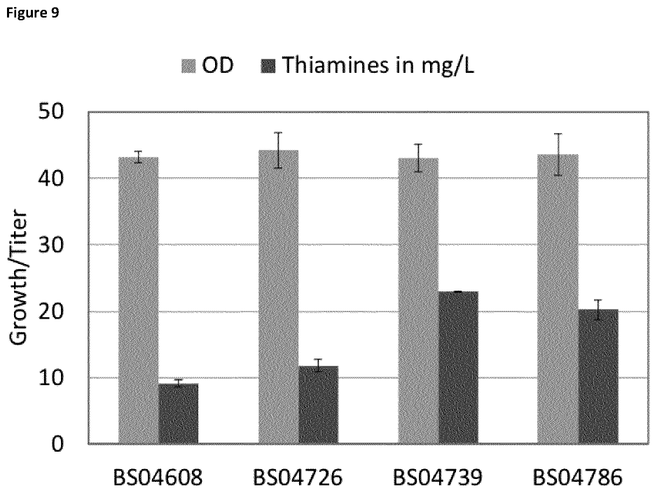

A 1 ml glycerol stock of BS04608, BS04726, BS04739 and BS04786 were inoculated into four separate 250 ml shakeflasks containing 50 ml mMOPS medium with ampicillin and kanamycin. The shake flasks were incubated at 37° C. at 250 rpm shaking for 20 hours resulting in an optical density of COD₆₀₀ 2.5-3.5. 0.9 ml B medium supplemented with ampicillin and kanamycin was added to a BioLector 32 well microfluidic plate equipped with pH, DO and biomass fluorescence (m2p-labs). 5% (v/v) of each strain culture was used to inoculate the B medium in four fermentation wells resulting in an initial cOD₆₀₀ 0.125-0.175 for each well and strain. The microfluidic plate was then sealed with gas-permeable sealing foil with evaporation reduction and loaded into the Biolector Pro (m2p-labs, Baesweiler, Germany). The temperature set to 37° C., pH controlled in each well to pH=7 by addition of 5 M NH₄OH, the agitation speed of the plate set to 1300 rpm (3 mm orbit) and the relative humidity in the chamber controlled at 85%. Four hours after inoculation the plate fermentations were paused, and each culture well was induced by addition of isopropyl β-D-1-thiogalactopyranoside (IPTG) to a concentration of 50 μM. The fermentation was resumed and following the depletion of glucose in the batch medium a fed batch phase was initiated by the addition of G medium to each culture well. This addition was controlled to an unlimited 1 μL pulse provided the DO % in each well registered greater than 70%. This transition took place between 6.7-11 h. The fermentations were terminated at 24 hours. An endpoint culture sample was taken from each fermentation well at 24 hours after inoculation. From this sample optical density (cOD₆₀₀) was measured according to analytical procedure I. Supernatants were obtained by spinning biomass down in a centrifuge at 4000 g, 5 min at 4° C. Supernatants were screened for thiamine using the bioassay described in analytical procedure II. The thiamine titre is shown in FIG. 9 and shows that the ThiC activity is approximately 2-fold greater in the strain without a functional CRP-cAMP complex (BS04876, BS04739) compared to the control strains with a functional CRP-cAMP complex having IscR mutant (BS04726) or WT (BS04608) genotype.

### Example 14—Engineering and Characterization of Genetically Modified E. coli Strains Capable of Enhanced Nicotinamide Riboside Production

The following strains of Escherichia coli from example 4 WO2020148351 are used.

TABLE 6
Strains

| Name | Description |
|---|---|
| BS1575 | BS1353 derivative with deletion of bioAFCDH |
| BS4260 | Derivative of BS1575 with a translation |
| | knockout of CRP (MAGE). |
| BS1575_NR | BS1575 transformed with pBS_NR |
| BS4260_NR | BS4260 transformed with pBS_NR |
| BS1575_NAM | BS1575 transformed with pBS_NAM |
| BS4260_NAM | BS4260 transformed with pBS_NAM |

The following plasmids are used.

TABLE 7
Plasmids

| Name | Description |
|---|---|
| pBS_NR | Plasmid (AmpR, p15A) comprising nadABCNadE*aphA genes SEQ ID NO: 140, |
| | 151, 156, 152, and 153 operably linked to a T5 LacO repressed promoter SEQ |
| | ID NO: 133 and a rrnB terminator SEQ ID NO: 137. Constitutive promoter SEQ |
| | ID NO: 136 and a rrnB terminator SEQ ID NO: 137. |
| pBS_NAM | Plasmid (AmpR, p15A) comprising nadA, nadB, nadC, nadE* and chi genes |
| | encoding SEQ ID NO: 140, 156, 152, 154, 155 operably linked to a |
| | T5 LacO repressed promoter SEQ ID NO: 133 and a rrnB terminator SEQ ID |
| | NO: 137. Constitutive promoter SEQ ID NO: 136 and a rrnB terminator SEQ ID |
| | NO: 137. |

Enhanced Nicotinamide Riboside (NR) Production in a CRP-cAMP Deficient Strain Overexpressing nadABC, nadE* and aphA Genes

The parent E. coli strain BS1575, and a mutant thereof lacking CRP (BS4260), are transformed with a plasmid (pBS_NR) comprising the genes nadABCE*aphA operatively linked an IPTG inducible promoter resulting in strains BS1575_NR and BS4260_NR. The genes expressed in plasmid pBS_NAM include:
E. coli nadA gene encoding quinolate synthase (NadA);
E. coli nadB encoding L-aspartate oxidase (NadB);
nadC encoding Nicotinate-nucleotide pyrophosphorylase (NadC);
aphA encoding Class B acid phosphatase (AphA); and
the Mannheimia succiniciproducens nadE gene encoding a polypeptide with nicotinic acid mononucleotide amidating activity (NadE*).

Cells of each strain are grown in 50 ml mMOPS medium, supplemented with 1 nmol/L biotin, 100 μg/mL ampicillin, at 37° C. and 250 rpm until the OD600 nm reaches 0.6. IPTG is then added to each culture to a final concentration of 0.064 mM that are then incubated for a further 6 h, and subsequently lysed.

NR, present in the recovered lysed cell supernatant, is measured by LC-MS using a 1290 Infinity series UHPLC coupled to a 6470 triple quadrupole from Agilent Technologies (Santa Clara, USA) (Ollagnier-de Choudens et al., 2005). NR production by an E. coli strain expressing the genes of the NR pathway (i.e. nadABCE*aphA), is enhanced when the host strain lacks the CRP-cAMP complex as compared to a strain expressing a gene encoding a native, CRP-cAMP complex.

Enhanced Nicotinamide (NAM) Production in a CRP-cAMP Deficient Strain Overexpressing nadABC, nadE* and NMN Nucleosidase (Chi) Genes.

For nicotinamide NAM production E. coli strain BS1575, and a mutant thereof lacking CRP (BS4260), are transformed with a plasmid (pBS_NAM) comprising the genes nadABCE* and chi operatively linked an IPTG inducible promoter, resulting in strains BS1575_NAM and BS4260_NAM. The genes expressed in plasmid pBS_NAM include:
E. coli nadA gene encoding quinolate synthase (NadA);
E. coli nadB encoding L-aspartate oxidase (NadB);
nadC encoding Nicotinate-nucleotide pyrophosphorylase (NadC);
chi encoding NMN nucleosidase (chi); and
Mannheimia succiniciproducens nadE* gene encoding a polypeptide with nicotinic acid mononucleotide amidating activity.

NAM production by an E. coli strain expressing the genes of the NAM pathway is enhanced when the host strain lacks the CRP-cAMP complex as compared to a strain expressing a gene encoding a native, CRP-cAMP complex.

### Example 15—Engineering and Characterization of Genetically Modified E. coli Strains Capable of Enhanced Production of Cobalamin

The following strains of Escherichia coli from example 5 WO2020148351 are used.

TABLE 8
Strains

| Name | Description |
|---|---|
| BS1575 | BS1353 derivative with deletion of bioAFCDH |
| BS1575_B12 | BS1575 derivative comprising an operon comprising |
| | cbiNQOM genes inserted into the genome |
| BS4260 | Derivative of BS1575 with a translation knockout |
| | of CRP (MAGE). |
| BS4260_B12 | BS4260 derivative comprising an operon comprising |
| | cbiNQOM genes inserted into the genome |
| BS1575_B12x3 | BS1575_12 transformed with pBS1748, |
| | pBS1749, pBS1750 |
| BS4260_B12x3 | BS4260_12 transformed with pBS1748, |
| | pBS1749, pBS1750 |

The following plasmids used in the example are listed below.

TABLE 9
Plasmids

| Name | Description |
|---|---|
| pBS1748 | Plasmid (AmpR, p15A) comprising CobG SEQ ID NO: 157 operably |
| | linked to a T5 LacO repressed promoter SEQ ID NO: 133 and a rrnB |
| | terminator SEQ ID NO: 137 and an operon of genes CobHIJLFK |
| | encoding SEQ ID NO: 1172, 168, 174, 173, 170, 1171 from a |
| | constitutive promoter apFAB309 SEQ ID NO: 1363 and a FAB |
| | terminator SEQ ID NO: 132. |
| pBS1749 | Plasmid (pBR322; KanR) comprising the operon of genes CobMNST |
| | encoding SEQ ID NO: 169, 175, 176 or 177 operably linked to a |
| | constitutive promoter apFAB309 SEQ ID NO: 136 and a FAB |
| | terminator SEQ ID NO: 132; second operon of genes CobROQBtuR |
| | SEQ ID NO: 178, 178, 179, 180, from a constitutive promoter |
| | apFAB309 SEQ ID NO: 136 and a FAB terminator SEQ ID NO: 132. |
| pBS1750 | Plasmid (pSC101, SpecR) comprising the operon of genes |
| | CobCDTPduX SEQ ID NO: 1184, 183, 185, 188 operably linked to a |
| | constitutive promoter apFAB309 SEQ ID NO: 136 and a FAB |
| | terminator SEQ ID NO: 132; second operon of genes CobUSCbiB SEQ |
| | ID NO: 182, 186, 187 from a constitutive promoter apFAB309 SEQ ID |
| | NO: 136 and a FAB terminator SEQ ID NO: 132. |

### Enhanced Cobalamin Production in a Strain Lacking CRP-cAMP Complex Overexpressing the Cobalamin Pathway Genes

Cobalamin is produced by E. coli cells expressing an IPTG inducible transgene encoding CobG and a constitutively expressing transgenes encoding transgenes encoding CobHIJLFK, CobMNST, CobCDTPduX, CobROQBtuR and CobUSCbiB (see table 9); and where the host E. coli cells further comprise the transgenes cbiNQOM inserted into their genome. E. coli strains BS1575_B12x3 and BS4260_B12x3 are cultured as described in WO2020148351. Cobalamin produced by the cultures is measured as follows: 2.5 mL of NaNO2 8% (w/v) and 2.5 mL of glacial acetic acid are added to 25 ml samples of each culture; which are then boiled for 30 min, and the resulting mixture filtered. Then 20 μL NaCN 10% (w/v) is added to 1 ml of aqueous phase; and 20 [symbol] L of resulting upper aqueous phase is injected into an HP1100 HPLC system (Agilent). NH2 column (4.6×250 mm2, 5 um) is employed for HPLC analysis with a flow rate of 1.7 mL/min and a wavelength of 360 nm, using a mobile phase of 250 mM phosphoric acid/acetonitrile (30/70, v/v). The production of cobalamin is enhanced when said host E. coli cells comprise the CRP-cAMP deficient strain (BS4260_B12x3) as compared to host E. coli cells comprising WT CRP-cAMP complex (BS1575_B12x3).

### Example 16—Engineering and Characterization of Genetically Modified E. coli Strains Capable of Enhanced Production of Pantothenate

The following strains of Escherichia coli are used.

TABLE 10
Strains

| Name | Description |
|---|---|
| BS1575 | BS1353 derivative with deletion of bioAFCDH |
| BS4260 | Derivative of BS1575 with a translation |
| | knockout of CRP (MAGE). |
| BS1575_B5 | BS1575 transformed with pBS_PAN |
| BS4260_B5 | BS1353 transformed with pBS_PAN |

The following plasmids used in the example are listed below.

TABLE 11
Plasmids

| Name | Description |
|---|---|
| pBS_PAN | Plasmid (p15A; AmpR) comprising a gene encoding ilvD SEQ ID NO: 194 |
| | operably linked to a T5 LacO repressed promoter SEQ ID NO: 133 and |
| | a rrnB terminator SEQ ID NO: 137. Second operon of genes PanBEC (SEQ |
| | ID NO: 205, 206, 207) from a constitutive promoter apFAB309 SEQ ID NO: |
| | 136 and a FAB terminator SEQ ID NO: 132. |

Enhanced Pantothenate Production in a Strain Lacking CRP-cAMP Complex Overexpressing ilvD, panB, panE and panC Genes

The parent E. coli strain BS1575, and a mutant thereof lacking CRP (BS4260), are transformed with a plasmid (pBS_PAN) comprising the genes ilvD, panB, panE and panC operably linked a constitutive promoter. The E. coli panB gene encodes a) 3-methyl-2-oxobutanoate hydroxymethyl-transferase; E. coli panE encodes 2-dehydropantoate 2-reductase and E. coli panC encodes pantothenate synthetase.

Cells of each strain are cultivated aerobically in 50 ml of NM1 Medium (NM1 composition: glucose, 20 g; (NH4) 2SO4, 20 g; KH2PO4, 2.0 g; MgSO4, 7H2O,0.4 g; NaCl, 1.6 g; yeast extract, 2 g; trace metal solution, supplemented with 1 nmol/L biotin, 100 μg/mL ampicillin at 31° C. and 250 rpm for 24 h. Pantothenate produced in the culture medium by each strain, following normalization for cell density, is measured as described in the methods section and the reference provided therein.

The production of pantothenate is enhanced in the CRP-cAMP deficient strain, BS1575_B₅, when co-expressing the transgene encoding ilvD and the transgenes encoding panBEC, as compared to their co-expression in the parent host E. coli strain expressing wild type CRP-cAMP complex (BS4260_B5).

### REFERENCES

Ifuku, O. et al. Molecular analysis of growth inhibition caused by overexpression of the biotin operon in Escherichia coli. Biosci Biotechnol Biochem 59, 184-189 (1995).
Deutscher, J., Francke, C. & Postma, P. W. How Phosphotransferase System-Related Protein Phosphorylation Regulates Carbohydrate Metabolism in Bacteria. Microbiol. Mol. Biol. Rev. 70, 939-1031 (2006).
Shimada, T., Fujita, N., Yamamoto, K. & Ishihama, A. Novel roles of camp receptor protein (CRP) in regulation of transport and metabolism of carbon sources. PLOS One 6, e20081 (2011).
Barth, E. et al. Interplay of cellular cAMP levels, {sigma}S activity and oxidative stress resistance in Escherichia coli. Microbiology 155, 1680-9 (2009).
Basak, S. & Jiang, R. Enhancing E. coli Tolerance towards Oxidative Stress via Engineering Its Global Regulator cAMP Receptor Protein (CRP). PLOS One 7, (2012).
Perrenoud, A. & Sauer, U. Impact of global transcriptional regulation by ArcA, ArcB, Cra, CRP, Cya, Fnr, and Mlc on glucose catabolism in Escherichia coli. J. Bacteriol. 187, 3171-3179 (2005).

1. A genetically modified host cell having increased production of one or more vitamin B compounds, wherein the host cell is genetically modified by
a) mutating one or more native polynucleotide constructs for reducing formation of a CRP-cAMP complex in the host cell and/or
b) introducing one or more genetic alterations increasing the degradation and/or non-CRP binding of cAMP in the host cell;
whereby the production of the vitamin B compound in the genetically modified host cell is increased compared to a parent host cell.

2. The host cell of claim 1, wherein the one or more mutated native polynucleotide constructs comprise one or more genes encoding one or more proteins selected from cAMP receptor protein (CRP), carbohydrate repression resistance protein (CRR) and adenylate cyclase protein (CyaA).

3. The host cell of claim 2, wherein the mutation is a deletion, disruption, and/or an attenuation of the gene.

4. The host cell of claim 3, wherein the mutation is a deletion through complete removal of the gene or a translational knockout by introducing one or more stop codons or frameshift mutations preventing expression of an active peptide.

5. The host cell of claim 3, wherein the deletion, disruption and/or attenuation comprise a translational knockout or a frameshift mutation.

6. The host cell of claim 3 or 5, wherein the deletion, disruption and/or attenuation is a point mutation in a promoter for the protein encoding sequence, in the RBS region and/or in protein encoding sequence.

7. The host cell of claim 6, wherein the point mutation is made in the sequence encoding the active site of the CyaA and reduces the activity of CyaA.

8. The host cell of claim 6, wherein the point mutation is made in the sequence encoding the cAMP binding moieties of CRP and reduced the affinity of CRP for cAMP.

9. The host cell of claims 2 to 8, wherein the
a) CRP is at least 70%, such as at least 80%, such as at least 90%, such as at least 95%, such as at least 96%, such as at least 97%, such as at least 98%, such as at least 99%, such as 100% identical to the CRP comprised in SEQ ID NO: 39 OR 97;
b) CRR is at least 70%, such as at least 80%, such as at least 90%, such as at least 95%, such as at least 96%, such as at least 97%, such as at least 98%, such as at least 99%, such as 100% identical to the CRR comprised in SEQ ID NO: 41 OR 99; and/or
c) CyaA is at least 70%, such as at least 80%, such as at least 90%, such as at least 95%, such as at least 96%, such as at least 97%, such as at least 98%, such as at least 99%, such as 100% identical to the CyaA comprised in SEQ ID NO: 43 OR 101.

10. The host cell of claims 2 to 8, wherein the gene encoding
a) the CRP is least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 40 or 98 or genomic DNA thereof encoding the CRP comprised in SEQ ID NO: 39 or 97;
b) the CRR is least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 42 or 100 or genomic DNA thereof encoding the CRR comprised in SEQ ID NO: 41 or 99; and
c) CyaA is least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 44 or 102 or genomic DNA thereof encoding the CyaA comprised in SEQ ID NO: 43 or 101.

11. The host cell of claim 9, wherein
a) the mutant CRP has a sequence comprised in SEQ ID NO: 39 OR 97 which has a mutation in one or more positions corresponding to T12, D138, T146, F69, R82, V139, G57, K58, E59, M60, 161, L62, S63, G72, E73, L74, R83, S84, T128, S129, A136, F137, Q171, E172, 1173, G174, Q175, 1176, V177, G178, C179, S180, R181, E182, T183, V184, G185, R186 of SEQ ID NO: 39 OR 97;
b) the mutant CRP has a sequence comprised in SEQ ID NO: 41 OR 99 which has a mutation in one or more positions corresponding to H76 and/or H91 of SEQ ID NO: 41 OR 99
c) the mutant CRP has a sequence comprised in SEQ ID NO: 39 OR 97 which has a mutation in one or more positions corresponding to G60, K59, L63, T65, R188, G195, K196, R192, S103, S113, D114, D116, W118, E185, T189, K260, K264, K332, W200, and/or D300 of SEQ ID NO: 43 OR 101.

12. The host cell of claim 11, wherein the mutation of CRP is one or more of T12N, D138V, T1461, F69C, R82C and V139M.

13. The host cell of claim 1, wherein the one or more genetic alterations increasing the non-CRP binding and/or degradation of cAMP in the host cell, comprise
a) introducing one or more heterologous non-CRP cAMP binding proteins or cAMP degrading enzymes into the host cell;
b) overexpressing one or more native cAMP binding proteins or cAMP degrading enzymes in the host cell; and/or
c) mutating one or more native non-CRP cAMP binding proteins or cAMP degrading enzymes in the host cell to increase their cAMP binding and/or degradation capability.

14. The host cell of claim 13, wherein the one or more non-CRP cAMP binding proteins and/or cAMP degrading enzymes are selected from cAMP phosphodiesterase (CpdA) and cAMP deaminase (CadD)

15. The host cell of claim 13 or 14, wherein the over-expression comprises a cis-modification in the genome or a trans-modification in a plasmid.

16. The host cell of claim 13 or 14, wherein the mutation is a point mutation in a promoter for the protein encoding sequence, in the RBS region and/or in protein encoding sequence.

17. The host cell of claim 16, wherein the point mutation is made in the sequence encoding the active site of the CpdA and or CadD and increases the activity of CpdA and/or CadD.

18. The host cell of claims 13 to 17, wherein the
a) CpdA is at least 70%, such as at least 80%, such as at least 90%, such as at least 95%, such as at least 96%, such as at least 97%, such as at least 98%, such as at least 99%, such as 100% identical to the CpdA comprised in SEQ ID NO: 45 or 103; and
b) CadD is at least 70%, such as at least 80%, such as at least 90%, such as at least 95%, such as at least 96%, such as at least 97%, such as at least 98%, such as at least 99%, such as 100% identical to the CadD comprised in SEQ ID NO: 47 or 105.

19. The host cell of claims 13 to 17, wherein the gene encoding:
a) the CpdA is least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 46 or genomic DNA thereof encoding the CpdA comprised in SEQ ID NO: 45 or 103; and
b) the CadD is least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 48 or genomic DNA thereof encoding the CadD comprised in SEQ ID NO: 47 or 105.

20. The host cell of any preceding claim, further comprising an operative metabolic pathway comprising one or more native or heterologous pathway elements producing the vitamin B compound.

21. The host cell of claim 20 wherein the one or more pathway elements comprise one or more FeS cluster dependent enzymes.

22. The host cell of claim 21, wherein the one or more pathway elements comprise one or more radical SAM enzymes.

23. The host cell of claims 20 to 22, wherein the vitamin B compound is biotin.

24. The host cell of claim 23, wherein the one or more pathway elements are selected from:
a) one or more fatty acid synthesis enzymes selected from FabH, FabG, FabA, FabZ, Fabl, FabB and FabF;
b) a malonyl-acyl carrier protein methyltransferase (BioC) converting Malonyl-acyl carrier protein to malonyl-acyl carrier protein methyl ester;
c) a pimelyl-acyl carrier protin methyl ester esterase (BioH) converting O-methylpimeloyl-acyl carrier protein to pimeloyl-acyl carrier protein;
d) a 8-amino-7-oxononanoate synthase (BioF) converting pimeloyl-acyl carrier protein to KAPA;
e) an adenosylmethionine-8-amino-7-oxononanoate transaminase (BioA) converting KAPA into DAPA using SAM as amino donor;
f) an adenosylmethionine-8-amino-7-oxononanoate transaminase (BioK) capable of converting KAPA into DAPA using lysine as amino donor;
g) a desthiobiotin synthase (BioD) converting DAPA to DTB;
h) a biotin biosynthesis cytochrome P450, (pimeloyl-[acp] synthase (Biol) converting long-chain acyl-[acyl-carrier protein to pimeloyl-[acp];
i) a 6-carboxyhexanoate-CoA ligase (BioW) converting pimelate+CoA to pimeloyl−CoA;
j) an FeS cluster Transcription factor polypeptide (IscR) capable of regulating an operon [isc operon] producing a FeS cluster co-factor; and
k) a biotin synthase (BioB) converting DTB to Biotin.

25. The host cell of claim 24, wherein the:
a) BioC has at least 70%, such at least 75%, such as at least 80%, such as at least 90%, such as at least 95%, such as at least 99%, such as 100% identity to the BioC comprised in SEQ ID NO: 1;
b) BioH has at least 70%, such at least 75%, such as at least 80%, such as at least 90%, such as at least 95%, such as at least 99%, such as 100% identity to the BioH comprised in SEQ ID NO: 3;
c) BioF has at least 70%, such at least 75%, such as at least 80%, such as at least 90%, such as at least 95%, such as at least 99%, such as 100% identity to the BioF comprised in SEQ ID NO: 5;
d) BioA has at least 70%, such at least 75%, such as at least 80%, such as at least 90%, such as at least 95%, such as at least 99%, such as 100% identity to the BioA comprised in SEQ ID NO: 7;
e) Biok has at least 70%, such at least 75%, such as at least 80%, such as at least 90%, such as at least 95%, such as at least 99%, such as 100% identity to the Biok comprised in SEQ ID NO: 9;
f) BioD has at least 70%, such at least 75%, such as at least 80%, such as at least 90%, such as at least 95%, such as at least 99%, such as 100% identity to the BioD comprised in SEQ ID NO: 11;
g) Biol has at least 70%, such at least 75%, such as at least 80%, such as at least 90%, such as at least 95%, such as at least 99%, such as 100% identity to the Biol comprised in SEQ ID NO: 13;
h) BioW has at least 70%, such at least 75%, such as at least 80%, such as at least 90%, such as at least 95%, such as at least 99%, such as 100% identity to the BioW comprised in SEQ ID NO: 15;
i) IscR has at least 70%, such at least 75%, such as at least 80%, such as at least 90%, such as at least 95%, such as at least 99%, such as 100% identity to the IscR comprised in SEQ ID NO: 17, 19, 21, 23, 25, 27, 29, 31, 33, or 35; and/or
j) BioB has at least 70%, such at least 75%, such as at least 80%, such as at least 90%, such as at least 95%, such as at least 99%, such as 100% identity to the T1BioB comprised in SEQ ID NO: 37.

26. The host cell of claims 24 to 41, wherein the one or more pathway elements are encoding by one or more genes selected from the group of:
a) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 2 or genomic DNA thereof encoding the BioC comprised in SEQ ID NO: 1;
b) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 4 or genomic DNA thereof encoding the BioH comprised in SEQ ID NO: 3;
c) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 6 or genomic DNA thereof encoding the BioF comprised in SEQ ID NO: 5;
d) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 8 or genomic DNA thereof encoding the BioA comprised in SEQ ID NO: 7;
e) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 10 or genomic DNA thereof encoding the Biok comprised in SEQ ID NO: 9;
f) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 12 or genomic DNA thereof encoding the BioD comprised in SEQ ID NO: 11;
g) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 14 or genomic DNA thereof encoding the Biol comprised in SEQ ID NO: 13;
h) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 16 or genomic DNA thereof encoding the BioW comprised in SEQ ID NO: 15;
i) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 18, 20, 22, 24, 26, 28, 30, 32, 34, or 36 or genomic DNA thereof encoding the IscR comprised in SEQ ID NO: 17, 19, 21, 23, 25, 27, 29, 31, 33, or 35 respectively; and
j) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 38 or genomic DNA thereof encoding the BioB comprised in SEQ ID NO: 37.

27. The host cell of claims 20 to 22, wherein the vitamin B compound is thiamine.

28. The host cell of claim 27, wherein the one or more pathway elements are selected from:
a) one or more phosphate synthase enzymes selected from phosphomethylpyrimidine synthase (ThiC); that catalyzes the synthesis of the hydroxymethylpyrimidine phosphate (HMP-P) moiety of thiamine from aminoimidazole ribotide (AIR) in a radical S-adenosyl-L-methionine (SAM)-dependent reaction;
b) a hydroxymethylpyrimidine/phosphomethylpyrimidine kinase (ThiD) that catalyzes the phosphorylation of hydroxymethylpyrimidine phosphate (HMP-P) to HMP-PP, and of HMP to HMP-P;
c) a sulfur carrier protein (ThiF) that catalyzes the adenylation of the carboxy terminus of ThiS and the subsequent displacement of AMP catalyzed by Thil-persulfide to give a ThiS-Thil acyl disulfide ThiS;
d) a thiamine diphosphate (ThiS) that catalyzes the adenylation of the carboxy terminus of ThiS and the subsequent displacement of AMP catalyzed by Thil-persulfide to give a ThiS-Thil acyl disulfide ThiS;
e) a 2-iminoacetate synthase (ThiH) converting that catalyzes the radical-mediated cleavage of tyrosine to 2-iminoacetate and 4-cresol;
f) a thiazole synthase (ThiG) that catalyzes the rearrangement of 1-deoxy-D-xylulose 5-phosphate (DXP) to produce the thiazole phosphate moiety of thiamine;
g) a hydroxyethylthiazole kinase (ThiM) that catalyzes the rearrangement of 1-deoxy-D-xylulose 5-phosphate (DXP) to produce the thiazole phosphate moiety of thiamine;
h) thiamine mono-phosphate (TMP) phosphatase that dephosphorylate thiamine monophosphate to thiamine;
i) a thiamine kinase (ThiK) that catalyzes the phosphorylation of thiamine to thiamine phosphate;
j) a thiamine-monophosphate kinase (ThiL) which catalyzes the ATP-dependent phosphorylation of thiamine-monophosphate (TMP) to form thiamine-pyrophosphate (TPP);
k) a thiamine-phosphate synthase (ThiE) that condenses 4-methyl-5-(beta-hydroxyethyl)-thiazole monophosphate (THZ-P) and 2-methyl-4-amino-5-hydroxymethyl pyrimidine pyrophosphate (HMP-PP) to form thiamine monophosphate (TMP);
l) a tRNA sulfurtransferase (Thil) that catalyzes the ATP-dependent transfer of a sulfur to tRNA to produce 4-thiouridine in position 8 of tRNAs;
m) an FeS cluster Transcription factor polypeptide (IscR) capable of regulating an operon [isc operon] producing a FeS cluster co-factor;
n) a glycine oxidase (ThiO) that catalyzes the FAD-dependent oxidative deamination of various amines and D-amino acids to yield the corresponding alpha-keto acids, ammonia/amine; hydrogen peroxide.

29. The host cell of claim 28, wherein the:
a) ThiC has at least 70%, such at least 75%, such as at least 80%, such as at least 90%, such as at least 95%, such as at least 99%, such as 100% identity to the ThiC comprised in SEQ ID NO: 51;
b) ThiD has at least 70%, such at least 75%, such as at least 80%, such as at least 90%, such as at least 95%, such as at least 99%, such as 100% identity to the ThiD comprised in SEQ ID NO: 53;
c) ThiF has at least 70%, such at least 75%, such as at least 80%, such as at least 90%, such as at least 95%, such as at least 99%, such as 100% identity to the ThiF comprised in SEQ ID NO: 55;
d) ThiS has at least 70%, such at least 75%, such as at least 80%, such as at least 90%, such as at least 95%, such as at least 99%, such as 100% identity to the ThiS comprised in SEQ ID NO: 57;
e) ThiH has at least 70%, such at least 75%, such as at least 80%, such as at least 90%, such as at least 95%, such as at least 99%, such as 100% identity to the ThiH comprised in SEQ ID NO: 59;
f) ThiG has at least 70%, such at least 75%, such as at least 80%, such as at least 90%, such as at least 95%, such as at least 99%, such as 100% identity to the ThiG comprised in SEQ ID NO: 61;
g) ThiM has at least 70%, such at least 75%, such as at least 80%, such as at least 90%, such as at least 95%, such as at least 99%, such as 100% identity to the ThiM comprised in SEQ ID NO: 63;
h) TMP phosphatase has at least 70%, such at least 75%, such as at least 80%, such as at least 90%, such as at least 95%, such as at least 99%, such as 100% identity to the TMP phosphatase comprised in SEQ ID NO: 65;
i) Thik has at least 70%, such at least 75%, such as at least 80%, such as at least 90%, such as at least 95%, such as at least 99%, such as 100% identity to the Thik comprised in SEQ ID NO: 67;
j) Thil has at least 70%, such at least 75%, such as at least 80%, such as at least 90%, such as at least 95%, such as at least 99%, such as 100% identity to the ThiL comprised in SEQ ID NO: 69;
k) ThiE has at least 70%, such at least 75%, such as at least 80%, such as at least 90%, such as at least 95%, such as at least 99%, such as 100% identity to the ThiE comprised in SEQ ID NO: 71;
l) Thil has at least 70%, such at least 75%, such as at least 80%, such as at least 90%, such as at least 95%, such as at least 99%, such as 100% identity to the Thil comprised in SEQ ID NO: 73;
m) IscR has at least 70%, such at least 75%, such as at least 80%, such as at least 90%, such as at least 95%, such as at least 99%, such as 100% identity to the IscR comprised in SEQ ID NO: 77, 79, 81, 83, 85, 87, 89, 91, 93 or 95; and/or
n) ThiO has at least 70%, such at least 75%, such as at least 80%, such as at least 90%, such as at least 95%, such as at least 99%, such as 100% identity to ThiO comprised in SEQ ID NO: 109, 111, 113 or 115.

30. The host cell of claims 27 to 29, wherein the one or more pathway elements are encoding by one or more genes selected from the group of:
a) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 2 or genomic DNA thereof encoding the ThiC comprised in SEQ ID NO: 51;
b) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 54 or genomic DNA thereof encoding the ThiD comprised in SEQ ID NO: 53;
c) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 56 or genomic DNA thereof encoding the ThiF comprised in SEQ ID NO: 55;
d) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 58 or genomic DNA thereof encoding the ThiS comprised in SEQ ID NO: 57;
e) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 60 or genomic DNA thereof encoding the ThiH comprised in SEQ ID NO: 59;
f) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 62 or genomic DNA thereof encoding the ThiG comprised in SEQ ID NO: 61;
g) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 64 or genomic DNA thereof encoding the ThiM comprised in SEQ ID NO: 63;
h) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 66 or genomic DNA thereof encoding the TMP phosphatase comprised in SEQ ID NO: 65;
i) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 68, or genomic DNA thereof encoding the ThiK comprised in SEQ ID NO: 67;
j) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 70, or genomic DNA thereof encoding the ThiL comprised in SEQ ID NO: 69;
k) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 72, or genomic DNA thereof encoding the ThiE comprised in SEQ ID NO: 71;
l) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 72, or genomic DNA thereof encoding the Thil comprised in SEQ ID NO: 73;
m) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 76, 78, 80, 82, 84, 86, 88, 90, 92, 94 or 96, or genomic DNA thereof encoding the IscR comprised in SEQ ID NO: 75, 77, 79, 81, 83, 85, 87, 89, 91, 93 or 95; and/or
n) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 110, 112, 114 or 116, or genomic DNA thereof encoding the ThiO comprised in SEQ ID NO: 109, 111, 113 or 115.

31. The host cell of claims 20 to 22, wherein the vitamin B compound is one or vitamins in the vitamin B₃ complex.

32. The host cell of claim 31, wherein the one or more pathway elements are selected from:
a) NadA quinolate synthase (EC: 2.5.1.72);
b) NadE nicotinic acid mononucleotide amidase;
c) NMN nucleosidase (EC: 3.2.2.14);
d) pncA deamidase (EC: 3.5.1.19);
e) NadB aspartate oxidase (EC: 1.4.3.16);
f) NadC nicotinate-nucleotide pyrophosphorylase (EC: 2.4.2.19);
g) AphA Class B acid phosphatase; and/or
h) an FeS cluster Transcription factor polypeptide (IscR) capable of regulating an operon [isc operon] producing a FeS cluster co-factor.

33. The host cell of claim 32, wherein the:
a) NadA quinolate synthase has at least 70%, such at least 75%, such as at least 80%, such as at least 90%, such as at least 95%, such as at least 99%, such as 100% identity to the NadA quinolate synthase comprised in SEQ ID NO: 140, 141, 142, 143, 144, 145, 146, 147, 148, 149 or 150;
b) NadE nicotinic acid mononucleotide amidase has at least 70%, such at least 75%, such as at least 80%, such as at least 90%, such as at least 95%, such as at least 99%, such as 100% identity to the NadE nicotinic acid mononucleotide amidase comprised in SEQ ID NO: 152;
c) NMN nucleosidase has at least 70%, such at least 75%, such as at least 80%, such as at least 90%, such as at least 95%, such as at least 99%, such as 100% identity to the NMN nucleosidase comprised in SEQ ID NO: 155;
d) pncA deamidase has at least 70%, such at least 75%, such as at least 80%, such as at least 90%, such as at least 95%, such as at least 99%, such as 100% identity to the pncA deamidase comprised in SEQ ID NO: 154;
e) NadB aspartate oxidase has at least 70%, such at least 75%, such as at least 80%, such as at least 90%, such as at least 95%, such as at least 99%, such as 100% identity to the NadB aspartate oxidase comprised in SEQ ID NO: 151;
f) NadC nicotinate-nucleotide pyrophosphorylase has at least 70%, such at least 75%, such as at least 80%, such as at least 90%, such as at least 95%, such as at least 99%, such as 100% identity to the NadC nicotinate-nucleotide pyrophosphorylase comprised in SEQ ID NO: 157;
g) AphA Class B acid phosphatase has at least 70%, such at least 75%, such as at least 80%, such as at least 90%, such as at least 95%, such as at least 99%, such as 100% identity to the AphA Class B acid phosphatase comprised in SEQ ID NO: 153, and/or
h) IscR has at least 70%, such at least 75%, such as at least 80%, such as at least 90%, such as at least 95%, such as at least 99%, such as 100% identity to the IscR comprised in SEQ ID NO: 77, 79, 81, 83, 85, 87, 89, 91, 93 or 95.

34. The host cell of claims 20 to 22, wherein the vitamin B compound is vitamin B₅ or pantothenate.

35. The host cell of claim 34 wherein the one or more pathway elements are selected from:
a) IlvD dihydroxy-acid dehydratase (EC: 4.2.1.9);
b) PanB 3-methyl-2-oxobutanoate hydroxymethyltransferase;
c) PanE 2-dehydropantoate 2-reductase;
d) PanC Pantothenate synthetase; and/or
e) FeS cluster Transcription factor polypeptide (IscR) capable of regulating an operon [isc operon] producing a FeS cluster co-factor.

36. The host cell of claim 35 wherein the:
a) IlvD dihydroxy-acid dehydratase has at least 70%, such at least 75%, such as at least 80%, such as at least 90%, such as at least 95%, such as at least 99%, such as 100% identity to the IlvD dihydroxy-acid dehydratase comprised in SEQ ID NO: 194, 195, 196, 197, 198, 199, 200, 201, 202, 203 or 204;
b) PanB 3-methyl-2-oxobutanoate hydroxymethyltransferase has at least 70%, such at least 75%, such as at least 80%, such as at least 90%, such as at least 95%, such as at least 99%, such as 100% identity to the IlvD dihydroxy-acid dehydratase comprised in SEQ ID NO: 205;
c) PanE 2-dehydropantoate 2-reductase has at least 70%, such at least 75%, such as at least 80%, such as at least 90%, such as at least 95%, such as at least 99%, such as 100% identity to the IlvD dihydroxy-acid dehydratase comprised in SEQ ID NO: 206; and/or
d) PanC Pantothenate synthetase has at least 70%, such at least 75%, such as at least 80%, such as at least 90%, such as at least 95%, such as at least 99%, such as 100% identity to the IlvD dihydroxy-acid dehydratase comprised in SEQ ID NO: 207; and/or
e) IscR has at least 70%, such at least 75%, such as at least 80%, such as at least 90%, such as at least 95%, such as at least 99%, such as 100% identity to the IscR comprised in SEQ ID NO: 77, 79, 81, 83, 85, 87, 89, 91, 93 or 95.

37. The host cell of claims 20 to 22, wherein the vitamin B compound is vitamin B₁₂ cobalamin.

38. The host cell of claim 37 wherein the one or more pathway elements are selected from:
a) CobG precorrin-3B synthase (EC: 1.14.13.83);
b) Cobl precorrin-2 C20-methyltransferase (EC: 2.1.1.130), converting precorrin-2 into precorrin-3A;
c) CobM precorrin-3 methylase (EC: 2.1.1.133) catalyzing the synthesis of precorrin-5 from precorrin-4;
d) CobF cobalt-precorrin-6A synthase (EC: 2.1.1.195) catalyzing the synthesis of precorrin-6A from precorrin-5B;
e) CobK precorrin-6A reductase (EC: 1.3.1.54) catalyzing the synthesis of precorrin-6B from precorrin-6A;
f) CobH precorrin isomerase (EC: 5.4.99.61) catalyzing the conversion of precorrin-8X to hydrogenobyrinate;
g) CobL Precorrin-6Y C (5,15)-methyltransferase (EC: 2.1.1.132) catalyzing the conversion of C-5 and C-15 in precorrin-6Y to form precorrin-8X;
h) CobJ Precorrin-3B C (17)-methyltransferase (EC: 2.1.1.131) catalyzing the methylation of precorrin-3B to form precorrin-4;
i) CobN subunit of aerobic cobaltochelatase (EC: 6.6.1.2) catalyzing cobalt insertion in the corrin ring;
j) CobS subunit of aerobic cobaltochelatase (EC: 6.6.1.2) catalyzing cobalt insertion in the corrin ring;
k) CobT subunit of aerobic cobaltochelatase (EC: 6.6.1.2) catalyzing cobalt insertion in the corrin ring;
l) CobR 4-hydroxyphenylacetate 3-monooxygenase (EC: 1.14.14.9);
m) CobO corrinoid adenosyltransferase (EC: 2.5.1.17) synthesizing adenosylcobalamin from cob (II) yrinate a,c-diamide;
n) CobQ cobyric acid synthase (EC: 6.3.5.10) catalyzing aminidations of adenosylcobyrinic A,C-diamide;
o) BtuR corrinoid adenosyltransferase (EC: 2.5.1.17);
p) CobU bifunctional adenosylcobalamin biosynthesis 4-hydroxyphenylacetate 3-monooxygenase activity (EC: 2.7.1.156);
q) CobD threonine-phosphate decarboxylase (EC: 4.1.1.81) decarboxylating L-threonine-O-3-phosphate to yield (R)-1-amino-2-propanol O-2-phosphate, the precursor for the linkage between the nucleotide loop and the corrin ring in cobalamin;
r) CobC adenosylcobalamin/alpha-ribazole phosphatase (EC: 3.1.3.73) converting adenosylcobalamin 5′-phosphate to adenosylcobalamin;
s) CobT polypeptide having Nicotinate-nucleotide-dimethylbenzimidazole phosphoribosyltrans-ferase (EC: 2.4.2.21);
t) CobS adenosylcobinamide-GDP ribazoletransferase (EC: 2.7.8.26);
u) CbiB cobalamin biosynthase (EC: 6.3.1.10) converting cobyric acid into cobinamide;
v) PduX L-threonine kinase (EC: 2.7.1.177) converting L-threonine to L-threonine-O-3-phosphate;
w) CbiN cobalt transport protein;
x) ChiQ cobalt transport protein;
y) CbiM cobalt transport protein;
z) CbiO cobalt import ATP-binding protein (EC: 3.6.3 . . . ); and/or
aa) FeS cluster Transcription factor polypeptide (IscR) capable of regulating an operon [isc operon] producing a FeS cluster co-factor.

39. The host cell of claim 38 wherein the:
a) CobG precorrin-3B synthase has at least 70%, such at least 75%, such as at least 80%, such as at least 90%, such as at least 95%, such as at least 99%, such as 100% identity to the CobG precorrin-3B synthase comprised in SEQ ID NO: 157, 158, 159, 160, 161, 162, 163, 164, 165, 166 or 167;
b) Cobl has at least 70%, such at least 75%, such as at least 80%, such as at least 90%, such as at least 95%, such as at least 99%, such as 100% identity to the Cobl comprised in SEQ ID NO: 168;
c) CobM has at least 70%, such at least 75%, such as at least 80%, such as at least 90%, such as at least 95%, such as at least 99%, such as 100% identity to the CobM comprised in SEQ ID NO: 169;
d) CobF has at least 70%, such at least 75%, such as at least 80%, such as at least 90%, such as at least 95%, such as at least 99%, such as 100% identity to the CobF comprised in SEQ ID NO: 170;
e) CobK has at least 70%, such at least 75%, such as at least 80%, such as at least 90%, such as at least 95%, such as at least 99%, such as 100% identity to the CobK comprised in SEQ ID NO: 171;
f) CobH has at least 70%, such at least 75%, such as at least 80%, such as at least 90%, such as at least 95%, such as at least 99%, such as 100% identity to the CobH comprised in SEQ ID NO: 172;
g) CobL has at least 70%, such at least 75%, such as at least 80%, such as at least 90%, such as at least 95%, such as at least 99%, such as 100% identity to the CobL comprised in SEQ ID NO: 173;
h) CobJ has at least 70%, such at least 75%, such as at least 80%, such as at least 90%, such as at least 95%, such as at least 99%, such as 100% identity to the CobJ comprised in SEQ ID NO: 174;
i) CobN has at least 70%, such at least 75%, such as at least 80%, such as at least 90%, such as at least 95%, such as at least 99%, such as 100% identity to the CobN comprised in SEQ ID NO: 175;
j) CobS has at least 70%, such at least 75%, such as at least 80%, such as at least 90%, such as at least 95%, such as at least 99%, such as 100% identity to the CobS comprised in SEQ ID NO: 176;
k) CobT has at least 70%, such at least 75%, such as at least 80%, such as at least 90%, such as at least 95%, such as at least 99%, such as 100% identity to the CobT comprised in SEQ ID NO: 177;
l) CobR has at least 70%, such at least 75%, such as at least 80%, such as at least 90%, such as at least 95%, such as at least 99%, such as 100% identity to the CobR comprised in SEQ ID NO: 178;
m) CobO has at least 70%, such at least 75%, such as at least 80%, such as at least 90%, such as at least 95%, such as at least 99%, such as 100% identity to the CobO comprised in SEQ ID NO: 179;
n) CobQ has at least 70%, such at least 75%, such as at least 80%, such as at least 90%, such as at least 95%, such as at least 99%, such as 100% identity to the CobQ comprised in SEQ ID NO: 180;
o) BtuR has at least 70%, such at least 75%, such as at least 80%, such as at least 90%, such as at least 95%, such as at least 99%, such as 100% identity to the BtuR comprised in SEQ ID NO: 181;
p) CobU has at least 70%, such at least 75%, such as at least 80%, such as at least 90%, such as at least 95%, such as at least 99%, such as 100% identity to the CobU comprised in SEQ ID NO: 182;
q) CobD has at least 70%, such at least 75%, such as at least 80%, such as at least 90%, such as at least 95%, such as at least 99%, such as 100% identity to the CobD comprised in SEQ ID NO: 183;
r) CobC has at least 70%, such at least 75%, such as at least 80%, such as at least 90%, such as at least 95%, such as at least 99%, such as 100% identity to the CobC comprised in SEQ ID NO: 184;
s) CobT has at least 70%, such at least 75%, such as at least 80%, such as at least 90%, such as at least 95%, such as at least 99%, such as 100% identity to the CobT comprised in SEQ ID NO: 185;
t) CobS has at least 70%, such at least 75%, such as at least 80%, such as at least 90%, such as at least 95%, such as at least 99%, such as 100% identity to the CobS comprised in SEQ ID NO: 186;
u) CbiB has at least 70%, such at least 75%, such as at least 80%, such as at least 90%, such as at least 95%, such as at least 99%, such as 100% identity to the CbiB comprised in SEQ ID NO: 187;
v) PduX has at least 70%, such at least 75%, such as at least 80%, such as at least 90%, such as at least 95%, such as at least 99%, such as 100% identity to the PduX comprised in SEQ ID NO: 188;
w) CbiN has at least 70%, such at least 75%, such as at least 80%, such as at least 90%, such as at least 95%, such as at least 99%, such as 100% identity to the CbiN comprised in SEQ ID NO: 189;
x) CbiQ has at least 70%, such at least 75%, such as at least 80%, such as at least 90%, such as at least 95%, such as at least 99%, such as 100% identity to the CbiQ comprised in SEQ ID NO: 190;
y) CbiM has at least 70%, such at least 75%, such as at least 80%, such as at least 90%, such as at least 95%, such as at least 99%, such as 100% identity to the CbiM comprised in SEQ ID NO: 191;
z) CbiO has at least 70%, such at least 75%, such as at least 80%, such as at least 90%, such as at least 95%, such as at least 99%, such as 100% identity to the CbiO comprised in SEQ ID NO: 192; and/or
aa) IscR has at least 70%, such at least 75%, such as at least 80%, such as at least 90%, such as at least 95%, such as at least 99%, such as 100% identity to the IscR comprised in SEQ ID NO: 77, 79, 81, 83, 85, 87, 89, 91, 93 or 95.

40. The host cell of claims 23 to 39 wherein the IscR factor is a mutant polypeptide having has at least one amino acid substitution selected from the group consisting of L15X, C92X, C98X, C104X, and H107X; wherein X is any amino acid other than the corresponding amino acid residue in the IscR comprised in SEQ ID NO: 17.

41. The method of claim 40, wherein the at least one amino acid substitution in the mutant IscR polypeptide is selected from the group consisting of:
a) L15X, wherein X is any one of F, Y, M and W;
b) C92X, wherein X is any one of Y, A, M, F and W;
c) C98X, wherein X is any one of A, V, I, L, F and W;
d) C104X, wherein X is any one of AV, I, L, F and W; and
e) H107X; wherein X, is any one of A, Y, V, I, and L.

42. The host cell of any preceding claim, wherein one or more genes and/or polypeptides of the pathway for the vitamin B compound are heterologous to the host cell.

43. The host cell of any preceding claim, further comprising at least 2 copies of one or more genes and/or polypeptides of the pathway for the vitamin B compound.

44. The host cell of any preceding claim, further comprising a transporter molecule facilitating transport of a precursor for or a product of the pathway for the vitamin B compound.

45. The host cell of any preceding claim, wherein the host cell is further genetically modified to provide an increased amount of a substrate in the pathway for the vitamin B compound.

46. The host cell of any preceding claim, wherein the host cell is further genetically modified to exhibit increased tolerance towards one or more substrates, intermediates, or products in the pathway for the vitamin B compound.

47. The host cell of any preceding claim, wherein one or more additional native or endogenous genes of the host cell are deleted, disrupted and/or attenuated.

48. The host cell of any preceding claim, wherein one or more genes in the pathway for the vitamin B compound are overexpressed.

49. The host cell of any preceding claim, wherein the host cell is prokaryotic or eukaryotic.

50. The host cell of claim 49, wherein the host cell is prokaryotic and of a genus selected from the group consisting of Escherichia, Bacillus, Brevibacterium, Burkholderia, Campylobacter, Corynebacterium, Serratia, Lactobacillus, Lactococcus, Acinetobacter, Acetobacter and Pseudomonas.

51. The host cell of claim 50, wherein the host cell is of the genus Escherichia, Corynebacterium, Bacillus, Serratia, Pseudomonas.

52. The host cell of claim 51, wherein the host cell is selected from the species Escherichia coli, Corynebacterium glutamicum, Bacillus subtilis, Serratia marcescens, Pseudomonas putida and/or Pseudomonas mutabilis.

53. The host cell of claim 49, wherein the host cell is eukaryotic and selected from the group consisting of mammalian, insect, plant, fungal or archaeal cells.

54. The host cell of claim 53, wherein the host cell is a fungal cell of a genus selected from Saccharomyces, Pichia and/or Ashbya.

55. The host cell of claim 54, wherein the host cell is selected from the species Saccharomyces cerevisiae, Pichia pastoris and/or Ashbya gossypii.

56. A mutated a polypeptide selected from the group consisting of a polypeptide which is at least 90% identical to:
a) the mutant CRP having a sequence comprised in SEQ ID NO: 39 or 97 and further comprising one or more mutations in positions corresponding to T12, D138, T146, F69, R82, V139, G57, K58, E59, M60, 161, L62, S63, G72, E73, L74, R83, S84, T128, S129, A136, F137, Q171, E172, 1173, G174, Q175, 1176, V177, G178, C179, S180, R181, E182, T183, V184, G185, and/or R186 of SEQ ID NO: 39 OR 97;
b) the mutant CRR having a sequence comprised in SEQ ID NO: 41 or 99 and further comprising one or more mutations in positions corresponding to H76 and/or H91 of SEQ ID NO: 41 OR 99; and/or
c) the mutant CyaA having a sequence comprised in SEQ ID NO: 43 or 101 and further comprising one or more mutations in positions corresponding to G60, K59, L63, T65, R188, G195, K196, R192, S103, S113, D114, D116, W118, E185, T189, K260, K264, K332, W200, and/or D300 of SEQ ID NO: 43 OR 101.

57. A polynucleotide construct comprising a polynucleotide sequence encoding a CRP, CRR or CyaA operably linked to one or more control sequences, wherein the polynucleotide construct comprises one or more mutations deleting, disrupting and/or attenuating the CRP, CRR or CyaA.

58. A polynucleotide construct comprising a polynucleotide sequence encoding a CpdA or CadD operably linked to one or more control sequences, wherein the polynucleotide construct comprises one or more mutations increasing the cAMP degradation activity of the encoded CpdA or CadD.

59. The polynucleotide construct of claim 57 or 58, wherein the construct is an expression vector.

60. The host cell of any preceding claim comprising the polynucleotide construct of claims 57 to 59.

61. A cell culture, comprising the host cell of any preceding claim and a growth medium.

62. A method for producing a vitamin B compound comprising
a) culturing the cell culture of claim 61 at conditions allowing the host cells to produce the vitamin B compound; and
b) optionally recovering and/or isolating the vitamin B compound.

63. The method of claim 62, further comprising feeding one or more exogenous vitamin B precursors to the host cell culture, optionally selected from the group of O-methylpimeloyl-acyl carrier protein, pimeloyl-acyl carrier protein, KAPA, DAPA, DTB and pimelate.

64. The method of claims 62 to 63, further comprising one or more elements selected from:
a) culturing the cell culture under aerobic or anaerobic conditions
b) cultivating the host cells under mixing;
c) cultivating the host cells at a temperature of between 25° C. to 50° C.;
d) cultivating the host cells at a pH of between 3-9; and
e) cultivating the host cells for between 10 hours to 120 days.

65. The method of claims 62 to 64, wherein the recovering and/or isolation step comprises separating a liquid phase of the cell culture from a solid phase of the cell culture to obtain a supernatant comprising the vitamin B compound and subjecting the supernatant to one or more steps selected from:
a) contacting the supernatant with one or more adsorbent resins in order to obtain at least a portion of the produced vitamin B compound, then optionally recovering the vitamin B compound from the resin in a concentrated solution prior to isolation of the vitamin B compound by crystallisation or solvent evaporation;
b) contacting the supernatant with one or more ion exchange or reversed-phase chromatography columns to obtain at least a portion of the vitamin B compound, then optionally recovering the vitamin B compound from the resin in a concentrated solution prior to isolation of the vitamin B compound by crystallisation or solvent evaporation;
c) extracting the vitamin B compound from the supernatant, such as by liquid-liquid extraction into an immiscible solvent, then optionally isolating the vitamin B compound by crystallisation or solvent evaporation; and
thereby recovering and/or isolating the vitamin B compound.

66. The method of claims 62 to 65, wherein the vitamin B compound yield is at least 10% higher such as at least 50%, such as at least 100%, such as least 150%, such as at least 200% higher than production by a host cell without reduced or eliminated CRP-cAMP complex formation and/or increased degradation and/or binding of cAMP.

67. The method of claims 62 to 66, wherein one or more steps of producing the vitamin B compound is performed in vitro.

68. The method of claims 62 to 64, further comprising mixing the vitamin B compound with one or more carriers, agents, adjuvants, additives and/or excipients, optionally pharmaceutical grade carriers, agents, adjuvants, additives and/or excipients.

69. A fermentation composition comprising the cell culture of claim 61 and the vitamin B compound.

70. The fermentation composition of claim 69, wherein at least 50%, such as at least 75%, such as at least 95%, such as at least 99% of the genetically modified host cells are lysed and/or disintegrated.

71. The fermentation composition of claims 69 to 70, wherein at least 50%, such as at least 75%, such as at least 95%, such as at least 99% of solid cellular material has separated from the composition.

72. The fermentation composition of claims 69 to 71, further comprising one or more compounds selected from:
a) precursor or products of the operative metabolic pathway producing the vitamin B compound;
b) supplemental nutrients; and
wherein the concentration of the vitamin B compound is at least 1 mg/L composition.

73. A composition comprising the fermentation composition of claims 69 to 72 and one or more carriers, agents, adjuvants, additives and/or excipients and at least trace amounts of one or more metabolites of the cell culture, optionally signature metabolites for the genetically modified host cell

74. The composition of claim 73, wherein the fermentation composition and the one or more carriers, agents, adjuvants, additives and/or excipients are in a dry solid form.

75. The composition of claim 73, wherein the fermentation composition and the one or more carriers, agents, adjuvants, additives and/or excipients are in a liquid stabilized form.

76. The composition of claim 74 or 75, refined into a pharmaceutical preparation, a dietary supplement, a cosmetic, a food preparation, a flavor preparation, a feed preparation and/or an analytical or diagnostic reagent.

77. The composition of claim 76, wherein the pharmaceutical preparation is in form of a powder, tablet, capsule, hard chewable and or soft lozenge or a gum.

78. The composition of claim 76, wherein the pharmaceutical preparation is in form of a liquid pharmaceutical solution.

79. The composition of claim 76 for use as a medicament.

80. The composition of claim 79 for use in the treatment of a nutritional deficiency.

81. A method for treating a disease in a mammal, comprising administering a therapeutically effective amount of the composition of claim 76 to the mammal.

82. The method of claim 81, wherein the disease is a nutritional deficiency or associated with a nutritional deficiency.
